(19) United States
(12) Patent Application Publication
(10) Pub. No.: US 2024/0018149 A1
(43) Pub. Date: Jan. 18, 2024

(54) PROTEIN DEGRADATION COMPOUNDS AND METHODS OF USE

(71) Applicants: Chu-Chiang CHEN, Taipei City (TW); Hung-Chuan CHEN, Taipei City (TW); Pei-Chin CHENG, Taipei City (TW); Chih-Chang CHOU, Taipei City (TW); AnHorn Medicines Co., Ltd., Taipei City (TW)

(72) Inventors: Chu-Chiang LIN, Taipei (CN); Hung-Chuan CHEN, New Taipei City (TW); Pei-Chin Xizhou, Township (TW); Chih-Chang CHOU, Taoyuan City (TW)

(21) Appl. No.: 18/216,566

(22) Filed: Jun. 29, 2023

Related U.S. Application Data
(60) Provisional application No. 63/357,054, filed on Jun. 30, 2022.

(51) Int. Cl. C07D 487/04; C07D 519/00

(52) U.S. Cl. CPC C07D 487/04; C07D 519/00

(57) ABSTRACT

This disclosure relates to bivalent compounds (e.g., bi-functional small molecule compounds), compositions comprising one or more of the bivalent compounds, and to methods of use the bivalent compounds for the degrading target proteins associated with a disease or condition.

### BACKGROUND OF THE DISCLOSURE

This disclosure relates to bivalent compounds (e.g., bi-functional small molecule compounds), compositions comprising one or more of the bivalent compounds, and to methods of use the bivalent compounds for the treatment of certain disease in a subject in need thereof.

### SUMMARY OF THE DISCLOSURE

One aspect of the present disclosure relates to a bivalent compound comprising a target binding moiety (TBM), and a cereblon E3 ubiquitin ligase binding moiety (CLM) represented by Formula (II)-1:

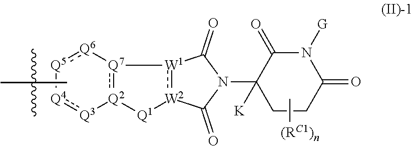

wherein:
[symbol] represents a single bond or a double bond;
W1 and W2 are each independently selected from C, CRC2 and N;
G is selected from the group consisting of H, OH, CH2OH, RC3OCOORC4, RC3OCONRC4RC5, and 2-(trimethylsilyl)ethoxymethyl group;
Q1 to Q7 are each independently C, O, S, N, CRC2 or NRC2; at least one of W1, W2, Q1, Q2, Q3, Q4, Q5, Q6 and Q7 comprises a heteroatom;
K is selected from the group consisting of H, an unsubstituted alkyl group, an alkyl group substituted by RC2, an unsubstituted cycloalkyl group, and a cycloalkyl group substituted by RC2; K is bound to the 6-membered ring with a stereospecific bond or a non-stereospecific bond;
RC1 is selected from the group consisting of an unsubstituted alkyl group, an alkyl group substituted by RC2, an unsubstituted aryl group, an aryl group substituted by RC2, an unsubstituted alkyl-aryl group, an alkyl-aryl group substituted by RC2, an unsubstituted alkoxy group, and an alkoxy group substituted by RC2;
RC2 is selected from the group consisting of H, halo, CH2OH, CRC4, NRC4RC5, 2-(trimethylsilyl)ethoxymethyl, an alkoxyl group, an unsubstituted alkyl group, an alkyl group substituted by one or more halo groups, an unsubstituted cycloalkyl group, a cycloalkyl group substituted by one or more halo groups, an unsubstituted aryl group, an aryl group substituted by one or more halo groups, an unsubstituted heteroaryl group, a heteroaryl group substituted by one or more halo groups, an unsubstituted heterocyclyl group, and a heterocyclyl group substituted by one or more halo groups;
RC3 is selected from the group consisting of an unsubstituted alkylene group, and an alkylene group substituted by RC2;
RC4 and RC5 are independently selected from the group consisting of an unsubstituted alkyl group, an alkyl group substituted by RC2, an unsubstituted cycloalkyl group, a cycloalkyl group substituted by RC2, an unsubstituted heterocyclyl group, a heterocyclyl group substituted by RC2, an unsubstituted aryl group, an aryl group substituted by RC2, an unsubstituted heteroaryl group, and a heteroaryl group substituted by RC2; and n is 0, 1, 2, 3 or 4, or a pharmaceutically acceptable salt or analog thereof, wherein the TBM is not selected from the group consisting of:

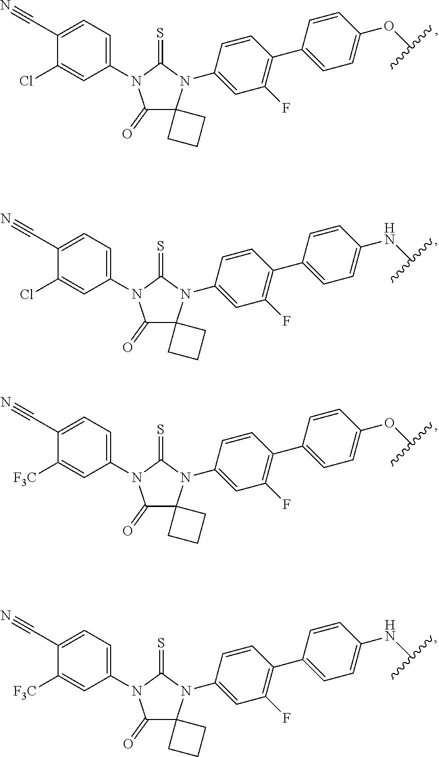 [SMILES: *Oc1ccc(-c2ccc(N3C(=S)N(c4ccc(C#N)c(Cl)c4)C(=O)C34CCC4)cc2F)cc1 ; *Nc1ccc(-c2ccc(N3C(=S)N(c4ccc(C#N)c(Cl)c4)C(=O)C34CCC4)cc2F)cc1 ; *Oc1ccc(-c2ccc(N3C(=S)N(c4ccc(C#N)c(C(F)(F)F)c4)C(=O)C34CCC4)cc2F)cc1 ; *Nc1ccc(-c2ccc(N3C(=S)N(c4ccc(C#N)c(C(F)(F)F)c4)C(=O)C34CCC4)cc2F)cc1]

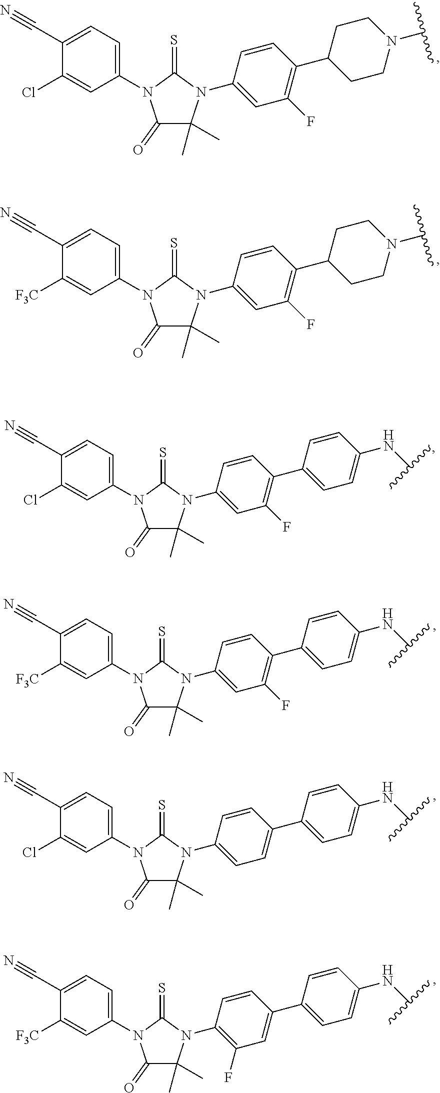 [SMILES: *N1CCC(c2ccc(N3C(=S)N(c4ccc(C#N)c(Cl)c4)C(=O)C3(C)C)cc2F)CC1 ; *N1CCC(c2ccc(N3C(=S)N(c4ccc(C#N)c(C(F)(F)F)c4)C(=O)C3(C)C)cc2F)CC1 ; *Nc1ccc(-c2ccc(N3C(=S)N(c4ccc(C#N)c(Cl)c4)C(=O)C3(C)C)cc2F)cc1 ; *Nc1ccc(-c2ccc(N3C(=S)N(c4ccc(C#N)c(C(F)(F)F)c4)C(=O)C3(C)C)cc2F)cc1 ; *Nc1ccc(-c2ccc(N3C(=S)N(c4ccc(C#N)c(Cl)c4)C(=O)C3(C)C)cc2)cc1 ; *Nc1ccc(-c2ccc(N3C(=S)N(c4ccc(C#N)c(C(F)(F)F)c4)C(=O)C3(C)C)c(F)c2)cc1]

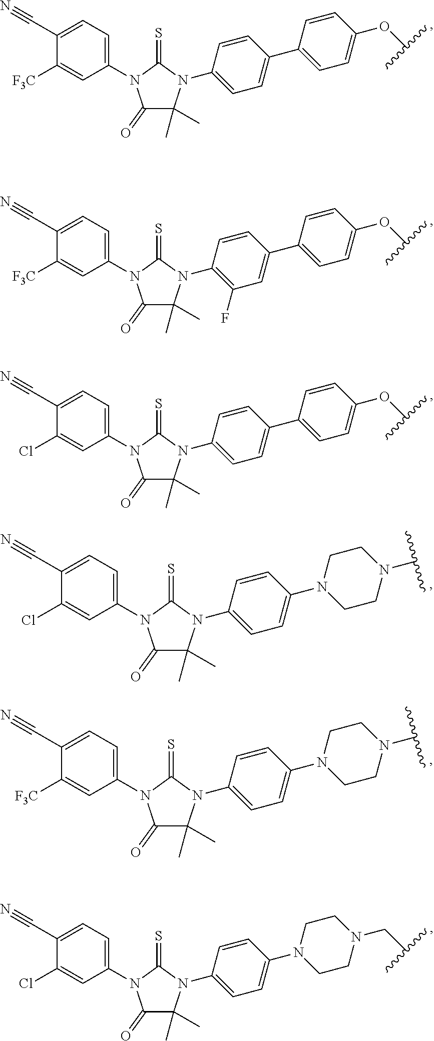 [SMILES: *Oc1ccc(-c2ccc(N3C(=S)N(c4ccc(C#N)c(C(F)(F)F)c4)C(=O)C3(C)C)cc2)cc1 ; *Oc1ccc(-c2ccc(N3C(=S)N(c4ccc(C#N)c(C(F)(F)F)c4)C(=O)C3(C)C)c(F)c2)cc1 ; *Oc1ccc(-c2ccc(N3C(=S)N(c4ccc(C#N)c(Cl)c4)C(=O)C3(C)C)cc2)cc1 ; *N1CCN(c2ccc(N3C(=S)N(c4ccc(C#N)c(Cl)c4)C(=O)C3(C)C)cc2)CC1 ; *N1CCN(c2ccc(N3C(=S)N(c4ccc(C#N)c(C(F)(F)F)c4)C(=O)C3(C)C)cc2)CC1 ; *CN1CCN(c2ccc(N3C(=S)N(c4ccc(C#N)c(Cl)c4)C(=O)C3(C)C)cc2)CC1]

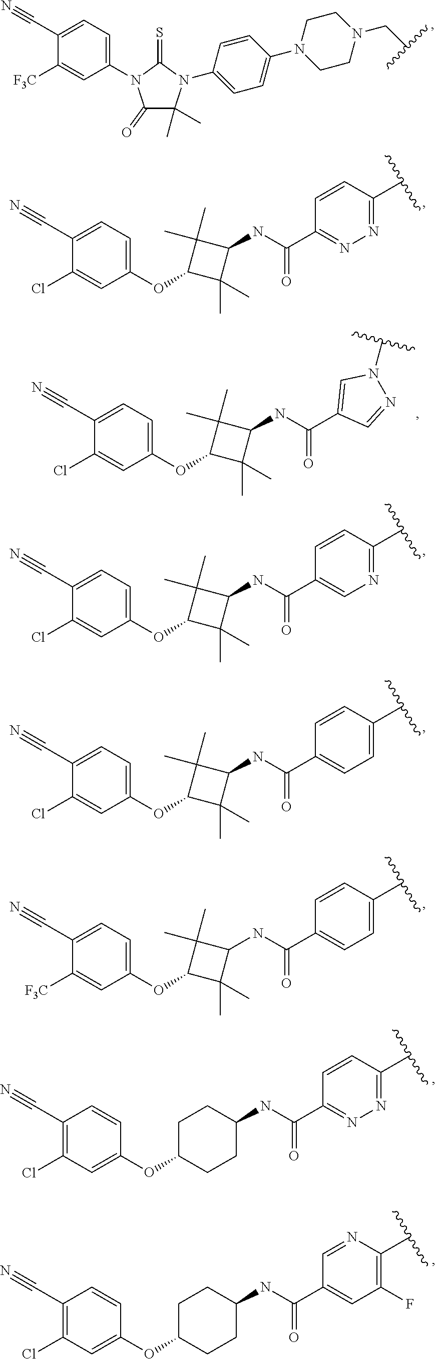

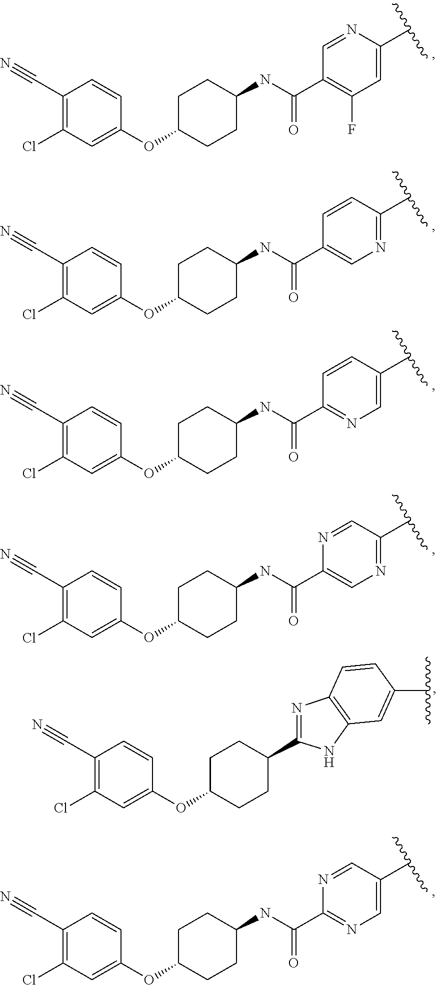 [SMILES: *c1cc(F)c(C(=O)N[C@H]2CC[C@H](Oc3ccc(C#N)c(Cl)c3)CC2)cn1 ; *c1ccc(C(=O)N[C@H]2CC[C@H](Oc3ccc(C#N)c(Cl)c3)CC2)cn1 ; *c1ccc(C(=O)N[C@H]2CC[C@H](Oc3ccc(C#N)c(Cl)c3)CC2)nc1 ; *c1cnc(C(=O)N[C@H]2CC[C@H](Oc3ccc(C#N)c(Cl)c3)CC2)cn1 ; *c1ccc2nc([C@H]3CC[C@H](Oc4ccc(C#N)c(Cl)c4)CC3)[nH]c2c1 ; *c1cnc(C(=O)N[C@H]2CC[C@H](Oc3ccc(C#N)c(Cl)c3)CC2)nc1]

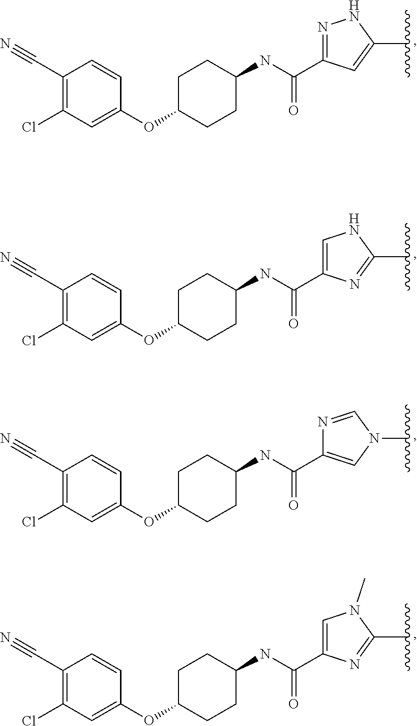 [SMILES: *c1cc(C(=O)N[C@H]2CC[C@H](Oc3ccc(C#N)c(Cl)c3)CC2)n[nH]1 ; *c1nc(C(=O)N[C@H]2CC[C@H](Oc3ccc(C#N)c(Cl)c3)CC2)c[nH]1 ; *n1cnc(C(=O)N[C@H]2CC[C@H](Oc3ccc(C#N)c(Cl)c3)CC2)c1 ; *c1nc(C(=O)N[C@H]2CC[C@H](Oc3ccc(C#N)c(Cl)c3)CC2)cn1C]

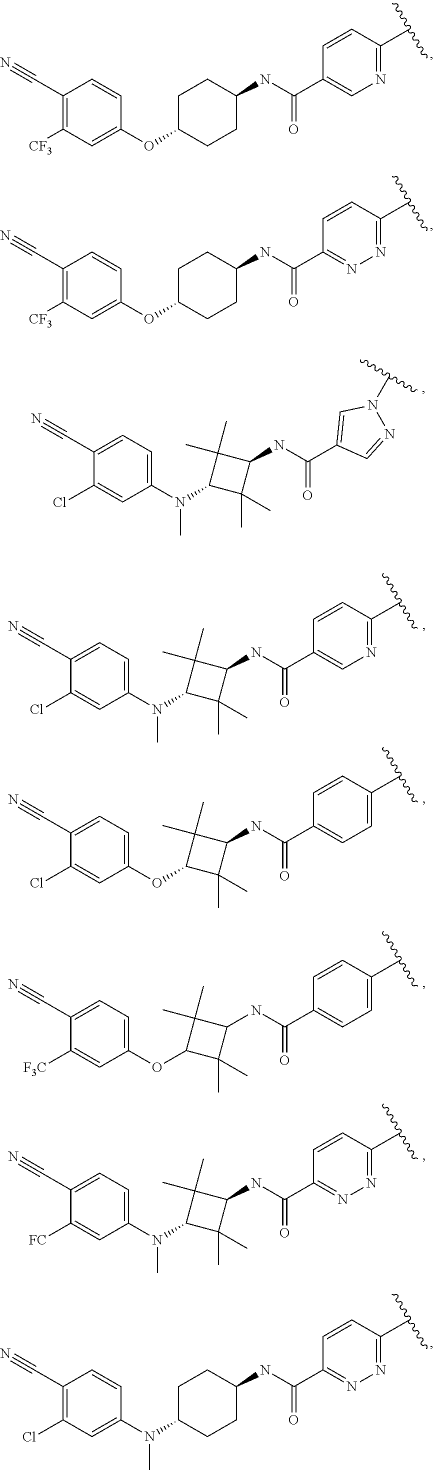

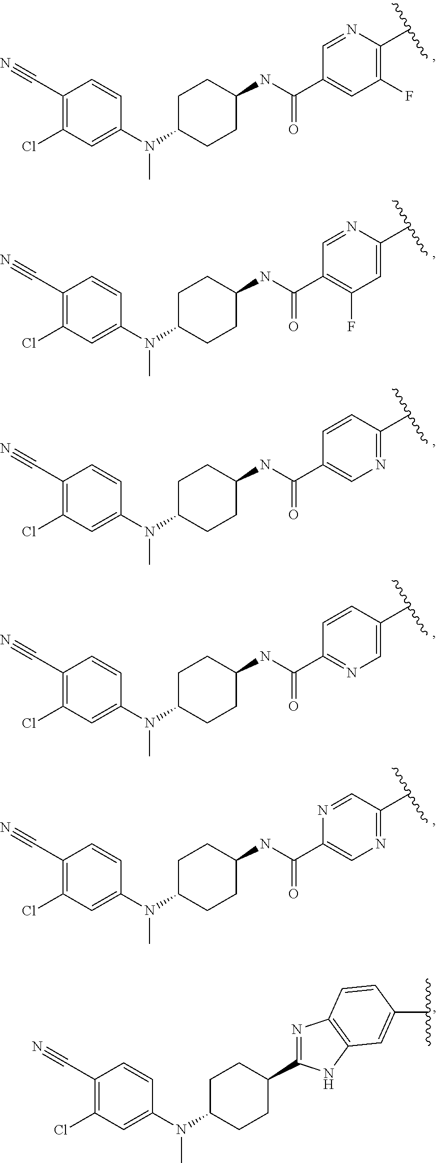 [SMILES: *c1ncc(C(=O)N[C@H]2CC[C@H](N(C)c3ccc(C#N)c(Cl)c3)CC2)cc1F ; *c1cc(F)c(C(=O)N[C@H]2CC[C@H](N(C)c3ccc(C#N)c(Cl)c3)CC2)cn1 ; *c1ccc(C(=O)N[C@H]2CC[C@H](N(C)c3ccc(C#N)c(Cl)c3)CC2)cn1 ; *c1ccc(C(=O)N[C@H]2CC[C@H](N(C)c3ccc(C#N)c(Cl)c3)CC2)nc1 ; *c1cnc(C(=O)N[C@H]2CC[C@H](N(C)c3ccc(C#N)c(Cl)c3)CC2)cn1 ; *c1ccc2nc([C@H]3CC[C@H](N(C)c4ccc(C#N)c(Cl)c4)CC3)[nH]c2c1]

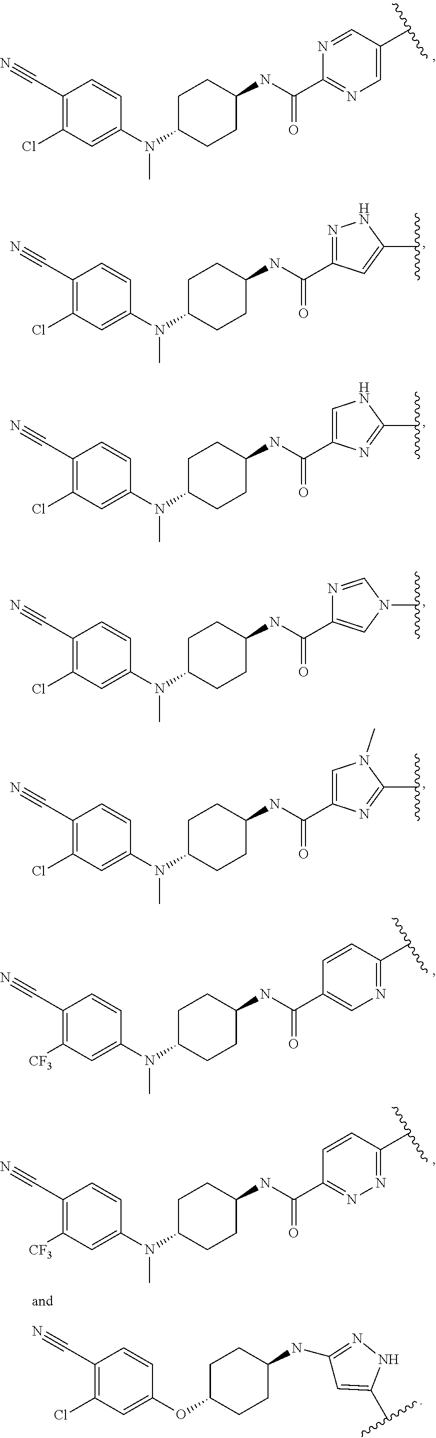

In some embodiments, the TBM is not selected from the group consisting of:

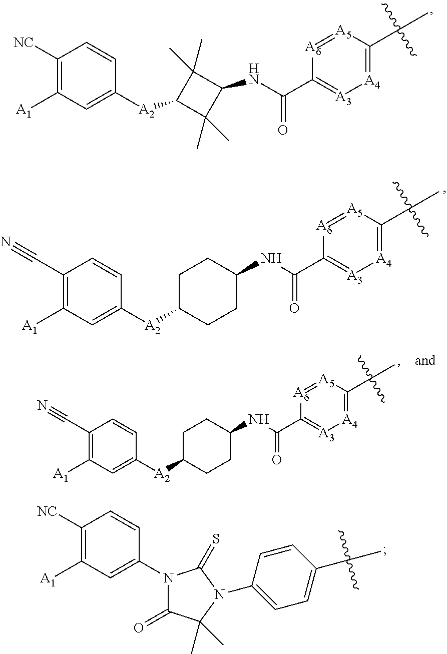

wherein A1 is selected from Cl, F, Br or CF3; A2 is selected from O, NH, N-methyl or N-ethyl; and A3, A4, A5 and A6 are each independently CH or N.

In some embodiments, the TBM is not selected from the group consisting of:

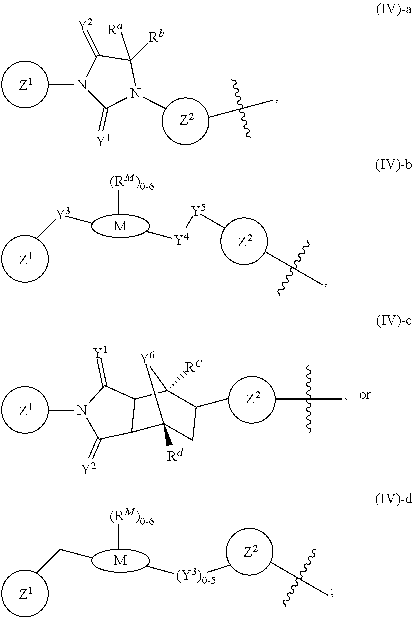

wherein Z1 is selected from the group consisting of an aryl group, a heteroaryl group, a bicyclic group, and a bi-heterocyclic group, each independently substituted by one or more substituents selected from the group consisting of a halo group, a hydroxyl group, a nitro group, CN, C≡CH, an unsubstituted C1-6 alkyl group, a C1-6 alkyl group substituted by a C1-6 alkoxyl group or one or more halo groups, an unsubstituted C1-6 alkoxyl group, a C1-6 alkoxyl group substituted by one or more halo groups, an unsubstituted C2-6 alkenyl, a C2-6 alkenyl substituted by one or more halo groups, an unsubstituted C2-6 alkynyl, and a C3-6 alkynyl substituted by one or more halo groups;
Y1, Y2, Y6 are each independently NRY1, O or S;
Y3, Y4, Y5 are each independently selected from the group consisting of a bond, O, NRY2, CRY1RY2, C═O, C═S, SO, SO2, a heteroaryl group, and an aryl group;
M is a 3- to 6-membered ring with 0 to 4 heteroatoms, which is unsubstituted or substituted by 1 to 6 RM groups;
each RM group is independently selected from the group consisting of H, an unsubstituted C1-6 alkyl group, a C1-6 alkyl group substituted by a C1-6 alkoxyl group or one or more halo groups, halogen, and a C1-6 alkoxy group; or two RM groups are taken together with the atom they are attached to and form a 3- to 8-membered ring system containing 0 to 2 heteroatoms;
Ra, Rb, Re, Rd, RY1, RY2 are each independently selected from the group consisting of H, an unsubstituted C1-6 alkyl group, a C1-6 alkyl group substituted by a C1-6 alkoxyl group or one or more halo groups, halogen, a C1-6 alkoxy group, a cyclic group, and a heterocyclic group; or Ra, Rb are taken together with the atom they are attached to and form a 3- to 8-membered ring system containing 0 to 2 heteroatoms;
Z2 is selected from the group consisting of a bond, a C1-6 alkyl group, a C1-6 heteroalkyl group, 0, an aryl group, a heteroaryl group, an alicyclic group, a heterocyclic group, a biheterocyclic group, a biaryl group, and a biheteroaryl group, each of which is unsubstituted or substituted by 1 to 10 RZ2 groups;
each R^{Z2} group is independently selected from the group consisting of H, halo, an unsubstituted C1-6 alkyl group, a C1-6 alkyl group substituted by one or more F, —ORZ2A, a C3-6 cycloalkyl group, a C4-6 cycloheteroalkyl group, an unsubstituted C1-6 alkyl group, a C1-6 alkyl group substituted by a C1-3 alkyl group or a C1-6 alkoxyl group or one or more halo groups, an unsubstituted heterocyclic group, a heterocyclic group substituted by a C1-3 alkyl group or a C1-6 alkoxyl group or one or more halo groups, an unsubstituted aryl group, an aryl group substituted by a C1-3 alkyl group or a C1-6 alkoxyl group or one or more halo groups, an unsubstituted heteroaryl group, a heteroaryl group substituted by a C1-3 alkyl group or a C1-6 alkoxyl group or one or more halo groups, a bicyclic hereoaryl group, an unsubstituted OC1-3 alkyl group, and a OC1-3 alkyl substituted by one or more groups selected from F, OH, NH₂, NRY1RY2 and CN; and
R^{Z2A} is selected from the group consisting of H, a C1-6 alkyl group, and a C1-6 heteroalkyl group, each of which is unsubstituted or substituted by a cycloalkyl group, a cycloheteroalkyl group, an aryl group, a heterocyclic group, a heteroaryl group, halo, or a OC1-3 alkyl group.

In some embodiments, the TBM is capable of binding to a protein degradable by a cereblon E3 ubiquitin ligase.

In some embodiment, Q1 is NRC2. In some embodiment, RC2 is an unsubstituted C1-6 alkyl group, a C1-6 alkyl group substituted by one or more halo groups, an unsubstituted C1-6 cycloalkyl group, a C1-6 cycloalkyl group substituted by one or more halo groups. In some embodiment, RC2 is an unsubstituted C1-6 alkyl group. In some embodiment, RC2 is an unsubstituted C1-4 alkyl group. In some embodiment, RC2 is methyl or ethyl.

In some embodiment, W1 and W2 are each C and connected together through a double bond.

In some embodiment, Q2, Q3, Q4, Q5, Q6 and Q7 together form a benzene or heterobenzen ring, optionally substituted with one or more RC2. In some embodiment, Q2, Q3, Q4, Q5, Q6 and Q7 together form an unsubstituted benzene or heterobenzen ring. In some embodiment, Q2, Q3, Q4, Q5, Q6 and Q7 together form an unsubstituted benzene ring.

In some embodiment, K is H.

In some embodiment, n is 0, 1, or 2. In some embodiment, n is 0 or 1. In some embodiment, n is 0.

In some embodiment, G is selected from the group consisting of H, CH2OH, RC3OCOORC4, and RC3OCONRC4RC5. In some embodiment, G is selected from the group consisting of H, CH2OH, and RC3OCOORC4. In some embodiment, G is H. In some embodiment, G is CH2OH. In some embodiment, G is RC3OCOORC4. In some embodiment, RC3 is selected from the group consisting of an unsubstituted alkylene group. In some embodiment, RC3 is an unsubstituted C1-6 alkylene group. In some embodiment, RC3 is an unsubstituted C1-4 alkylene group. In some embodiment, RC3 is methylene or ethylene. In some embodiment, RC3 is methylene. In some embodiment, RC4 is an unsubstituted alkyl group or an alkyl group substituted by RC2. In some embodiment, RC4 is an unsubstituted alkyl group. In some embodiment, RC4 is an unsubstituted C₁₋₆ alkyl group. In some embodiment, RC4 is an unsubstituted C1-4 alkyl group. In some embodiment, RC4 is methyl or ethyl. In some embodiment, RC4 is methyl.

In some embodiment, the CLM is represented with Formula (II)-2:

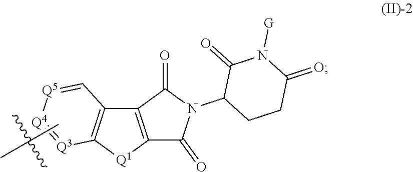

wherein
Q1 is 0, S or NRC2;
Q3 to Q5 are each independently C or CRC2;
G is selected from the group consisting of H, OH, CH2OH, RC3OCOORC4 and 2-(trimethylsilyl)ethoxymethyl group;
RC2 is selected from the group consisting of H, CH2OH, CRC4, 2-(trimethylsilyl)ethoxymethyl, an unsubstituted C1-6 alkyl group, a C1-6 alkyl group substituted by one or more halo groups, a unsubstituted C1-6 cycloalkyl group, a C1-6 cycloalkyl group substituted by one or more halo groups;
RC3 is selected from the group consisting of a methylene group and a methylene group substituted by RC2;
RC4 is selected from the group consisting of an unsubstituted alkyl group, an alkyl group substituted by RC2, an unsubstituted cycloalkyl group, a cycloalkyl group substituted by RC2, an unsubstituted heterocyclyl group, a heterocyclyl group substituted by RC2.

In some embodiment, Q1 is NRC2. In some embodiment, RC2 is an unsubstituted C1-6 alkyl group, a C1-6 alkyl group substituted by one or more halo groups, an unsubstituted C1-6 cycloalkyl group, a C1-6 cycloalkyl group substituted by one or more halo groups. In some embodiment, RC2 is an unsubstituted C1-6 alkyl group. In some embodiment, RC2 is an unsubstituted C1-4 alkyl group. In some embodiment, RC2 is methyl or ethyl.

In some embodiment, Q3-Q5 are each C.

In some embodiment, G is selected from the group consisting of H, CH2OH, RC3OCOORC4, and RC3OCONRC4RC5. In some embodiment, G is selected from the group consisting of H, CH2OH, and RC3OCOORC4. In some embodiment, G is H. In some embodiment, G is CH2OH. In some embodiment, G is RC3OCOORC4. In some embodiment, RC3 is selected from the group consisting of an unsubstituted alkylene group. In some embodiment, RC3 is an unsubstituted C1-6 alkylene group. In some embodiment, RC3 is an unsubstituted C1-4 alkylene group. In some embodiment, RC3 is methylene or ethylene. In some embodiment, RC3 is methylene. In some embodiment, RC4 is an unsubstituted alkyl group or an alkyl group substituted by RC2. In some embodiment, RC4 is an unsubstituted alkyl group. In some embodiment, RC4 is an unsubstituted C1-6 alkyl group. In some embodiment, RC4 is an unsubstituted C1-4 alkyl group. In some embodiment, RC4 is methyl or ethyl. In some embodiment, RC4 is methyl.

In some embodiment, the TBM is connected to the CLM through a bond or a linker moiety (L). In some embodiment, the TBM is connected to the CLM through a linker moiety (L). In some embodiment, the TBM is connected to the CLM through Q5. In some embodiment, the TBM is connected to the CLM through Q4. In some embodiment, the TBM is connected to the CLM through Q3.

In some embodiment, the linker moiety is of Formula (III):

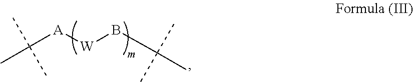

wherein
A, W, and B, at each occurrence, are independently selected from null, CO, CO₂, C(O)NR¹, C(S)NR¹, O, S, SO, SO₂, SO₂NR¹, NR¹, NR¹CO, NR¹CONR², NR¹C(S), optionally substituted C₁-C₈ alkyl, optionally substituted C₁-C₈ alkoxy, optionally substituted C₁-C₈alkoxyC₁-C₈alkyl, optionally substituted C₁-C₈ haloalkyl, optionally substituted C₁-C₈ hydroxyalkyl, optionally substituted C₂-C₈ alkenyl, optionally substituted C₂-C₈ alkynyl, optionally substituted C₃-C₈ cycloalkyl, optionally substituted C₃-C₈ cycloalkoxy, optionally substituted 3-8 membered heterocyclyl, optionally substituted aryl, optionally substituted heteroaryl, optionally substituted C₃-C₁₃ fused cycloalkyl, optionally substituted C₃-C₁₃ fused heterocyclyl, optionally substituted C₃-C₁₃ bridged cycloalkyl, optionally substituted C₃-C₁₃ bridged heterocyclyl, optionally substituted C₃-C₁₃ spiro cycloalkyl, and optionally substituted C₃-C₁₃ spiro heterocyclyl, wherein
   R¹ and R² are independently selected from hydrogen, optionally substituted C₁-C₆ alkyl, optionally substituted C₃-C₆ cycloalkyl, optionally substituted C₃-C₆ cycloalkoxy, optionally substituted 3-6 membered heterocyclyl, optionally substituted C₁-C₆ alkoxy, optionally substituted C₁-C₆ alkoxyalkyl, optionally substituted C₁-C₆ haloalkyl, optionally substituted C₁-C₆ hydroxyalkyl, optionally substituted C₁-C₆ alkylamino, and optionally substituted C₁-C₆alkylaminoC₁-C₆alkyl; and
m is 0 to 15.

In some embodiment, the linker moiety is of Formula (III)-1:

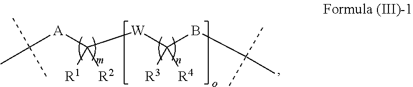

wherein
R¹, R², R³, and R⁴, at each occurrence, are independently selected from hydrogen, halogen, CN, OH, NH₂, optionally substituted C₁-C₆ alkyl, optionally substituted C₃-C₆ cycloalkyl, optionally substituted C₃-C₆ cycloalkoxy, optionally substituted 3-6 membered heterocyclyl, optionally substituted C₁-C₆ alkoxy, optionally substituted C₁-C₆ alkoxyalkyl, optionally substituted C₁-C₆ haloalkyl, optionally substituted C₁-C₆ hydroxyalkyl, optionally substituted C₁-C₆ alkylamino, and optionally substituted C1-C₆alkylaminoC₁-C₆alkyl;
A, W, and B, at each occurrence, are independently selected from null, CO, CO₂, C(O)NR⁵, C(S)NR⁵, O, S, SO, SO₂, SO₂NR⁵, NR⁵, NR⁵CO, NR⁵CONR⁶, NR⁵C(S), optionally substituted C₁-C₈ alkyl, optionally substituted C₁-C₈ alkoxy, optionally substituted C₁-C₈alkoxyC₁-C₈alkyl, optionally substituted C₁-C₈ haloalkyl, optionally substituted C₁-C₈ hydroxyalkyl, optionally substituted C₂-C₈ alkenyl, optionally substituted C₂-C₈ alkynyl, optionally substituted C₃-C₈ cycloalkyl, optionally substituted C₃-C₈ cycloalkoxy, optionally substituted 3-8 membered heterocyclyl, optionally substituted aryl, optionally substituted heteroaryl, optionally substituted C₃-C₁₃ fused cycloalkyl, optionally substituted C₃-C₁₃ fused heterocyclyl, optionally substituted C₃-C₁₃ bridged cycloalkyl, optionally substituted C₃-C₁₃ bridged heterocyclyl, optionally substituted C₃-C₁₃ spiro cycloalkyl, and optionally substituted C₃-C₁₃ spiro heterocyclyl, wherein
   R⁵ and R⁶ are independently selected from hydrogen, optionally substituted C₁-C₆ alkyl, optionally substituted C₃-C₆ cycloalkyl, optionally substituted C₃-C₆ cycloalkoxy, optionally substituted 3-6 membered heterocyclyl, optionally substituted C₁-C₆ alkoxy, optionally substituted C₁-C₆ alkoxyalkyl, optionally substituted C₁-C₆ haloalkyl, optionally substituted C₁-C₆ hydroxyalkyl, optionally substituted C₁-C₆ alkylamino, and optionally substituted C₁-C₆alkylaminoC₁-C₆alkyl;
m is 0 to 15;
each n is 0 to 15; and
o is 0 to 15.

In some embodiment, the linker moiety is of Formula (III)-2:

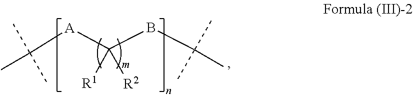

wherein
each R¹, and each R² are independently selected from hydrogen, halogen, CN, OH, NH₂, and optionally substituted C₁-C₆ alkyl, optionally substituted C₃-C₆ cycloalkyl, optionally substituted C₃-C₆ cycloalkoxy, optionally substituted 3-6 membered heterocyclyl, optionally substituted C₁-C₆ alkoxy, optionally substituted C₁-C₆ alkoxyalkyl, optionally substituted C₁-C₆ haloalkyl, optionally substituted C₁-C₆ hydroxyalkyl, optionally substituted C₁-C₆ alkylamino, or C₁-C₆alkylaminoC₁-C₆alkyl;
each A and each B are independently selected from null, CO, CO₂, C(O)NR³, C(S)NR³, O, S, SO, SO₂, SO₂NR³, NR³, NR³CO, NR³CONR⁴, NR³C(S), and optionally substituted C₁-C₈ alkyl, optionally substituted C₁-C₈ alkoxy, optionally substituted C₁-C₈alkoxyC₁-C₈alkyl, optionally substituted C₁-C₈ haloalkyl, optionally substituted C₁-C₈ hydroxyalkyl, optionally substituted C₂-C₈ alkenyl, optionally substituted C₂-C₈ alkynyl, optionally substituted C₃-C₈ cycloalkyl, optionally substituted C₃-C₈ cycloalkoxy, optionally substituted 3-8 membered heterocyclyl, optionally substituted aryl, optionally substituted heteroaryl, optionally substituted C₃-C₁₃ fused cycloalkyl, optionally substituted C₃-C₁₃ fused heterocyclyl, optionally substituted C₃-C₁₃ bridged cycloalkyl, optionally substituted C₃-C₁₃ bridged heterocyclyl, optionally substituted C₃-C₁₃ spiro cycloalkyl, or C₃-C₁₃ spiro heterocyclyl, wherein
R³ and R⁴ are independently selected from hydrogen, and optionally substituted C₁-C₆ alkyl, optionally substituted C₃-C₆ cycloalkyl, optionally substituted C₃-C₆ cycloalkoxy, optionally substituted 3-6 membered heterocyclyl, optionally substituted C₁-C₆ alkoxy, optionally substituted C₁-C₆ alkoxyalkyl, optionally substituted C₁-C₆ haloalkyl, optionally substituted C₁-C₆ hydroxyalkyl, optionally substituted C₁-C₆ alkylamino, or C₁-C₆alkylaminoC₁-C₆alkyl;
each m is 0 to 15; and
n is 0 to 15.

In some embodiment, the linker moiety is of FORMULA (III)-3:

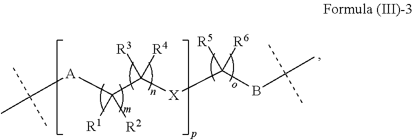

wherein
X is selected from O, NH, and NR⁷;
R¹, R², R³, R⁴, R⁵, and R⁶, at each occurrence, are independently selected from hydrogen, halogen, CN, OH, NH₂, optionally substituted C₁-C₆ alkyl, optionally substituted C₃-C₆ cycloalkyl, optionally substituted C₃-C₆ cycloalkoxy, optionally substituted 3-6 membered heterocyclyl, optionally substituted C₁-C₆ alkoxy, optionally substituted C₁-C₆ alkoxyalkyl, optionally substituted C₁-C₆ haloalkyl, optionally substituted C₁-C₆ hydroxyalkyl, optionally substituted C₁-C₆ alkylamino, and optionally substituted C1-C₆alkylaminoC₁-C₆alkyl; A and B, at each occurrence, are independently selected from null, CO, CO₂, C(O)NR⁷, C(S)NR⁷, O, S, SO, SO₂, SO₂NR⁷, NR⁷, NR⁷CO, NR⁷CONR⁸, NR⁷C(S), optionally substituted C₁-C₈ alkyl, optionally substituted C₁-C₈ alkoxy, optionally substituted C₁-C₈alkoxyC₁-C₈alkyl, optionally substituted C₁-C₈ haloalkyl, optionally substituted C₁-C₈ hydroxyalkyl, optionally substituted C₂-C₈ alkenyl, optionally substituted C₂-C₈ alkynyl, optionally substituted C₃-C₈ cycloalkyl, optionally substituted C₃-C₈ cycloalkoxy, optionally substituted 3-8 membered heterocyclyl, optionally substituted aryl, optionally substituted heteroaryl, optionally substituted C₃-C₁₃ fused cycloalkyl, optionally substituted C₃-C₁₃ fused heterocyclyl, optionally substituted C₃-C₁₃ bridged cycloalkyl, optionally substituted C₃-C₁₃ bridged heterocyclyl, optionally substituted C₃-C₁₃ spiro cycloalkyl, and optionally substituted C₃-C₁₃ spiro heterocyclyl, wherein
R⁸ and R⁸ are independently selected from hydrogen, optionally substituted C₁-C₆ alkyl, optionally substituted C₃-C₆ cycloalkyl, optionally substituted C₃-C₆ cycloalkoxy, optionally substituted 3-6 membered heterocyclyl, optionally substituted C₁-C₆ alkoxy, optionally substituted C₁-C₆ alkoxyalkyl, optionally substituted C₁-C₆ haloalkyl, optionally substituted C₁-C₆ hydroxyalkyl, optionally substituted C₁-C₆ alkylamino, and optionally substituted C1-C₆alkylaminoC₁-C₆alkyl;
each m is 0 to 15;
each n is 0 to 15;
is 0 to 15; and
p is 0 to 15.

In some embodiment, the linker moiety comprises a 3 to 13 membered ring, a 4 to 13 membered fused ring, a 5 to 13 membered bridged ring, and a 5 to 13 membered spiro ring. In some embodiment, the linker moiety comprises a ring selected from the group consisting of Formula C1, C2, C3, C4 and C5:

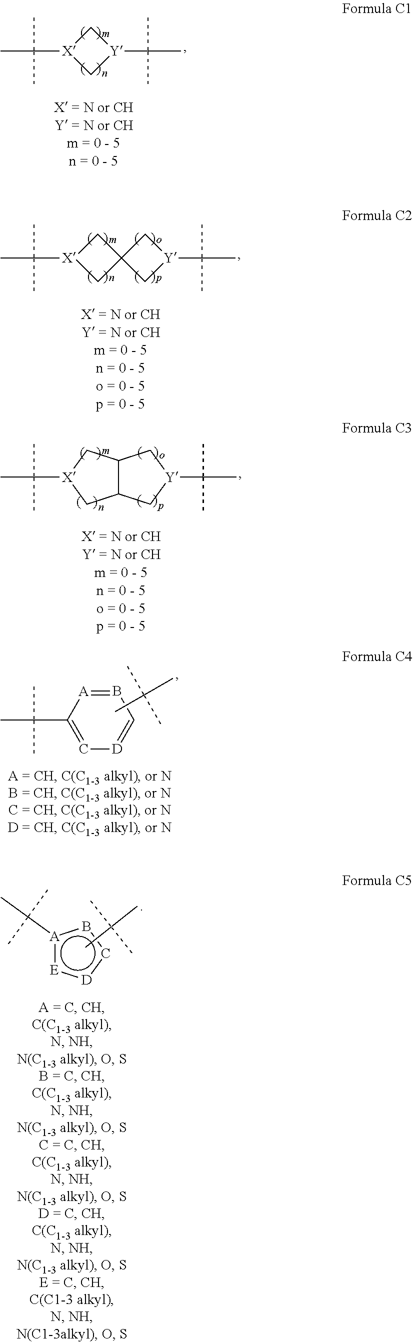

In some embodiment, the linker moiety is of Formula (IV):

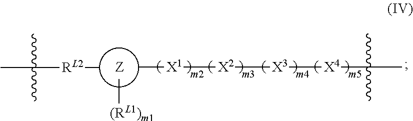

wherein
Z is selected from the group consisting of a 3- to 8-membered ring, a 5- to 12-membered bicyclic ring, an 8- to 15-membered tricyclic ring and a 6- to 12-membered spiro bicyclic ring, each independently having 0-4 heteroatoms;
RL1 is selected from the group consisting of H, an unsubstituted C₁₋₆ alkyl group, a C₁₋₆ alkyl group substituted by a C₁₋₆ alkoxyl group or one or more halo groups, halogen, a C₁₋₆ alkoxy group, a keto group, or an oxide group; wherein, two RL1 groups are optionally taken together to form a 3-8 membered ring system containing 0-2 heteroatoms;
RL2 is a bond or an ethynylene group;
X1 is selected from the group consisting of a methylene group and an ethylene group;
X2 and X4 are independently selected from the group consisting of a methylene group, C(═O), C(═O)C(═O), CONRL3, NRL3C(═O), NRL3C(═O)NRL4, NRL3C(═O)C(═O)NRL4, NRL3SO2NRL4, SO2NRL3, CRL3RL4, NRL5, O and S;
X3 is selected from the group consisting of an unsubstituted C1-8 alkylene group, a C1-8 alkylene group substituted by 1 to 6 RL1, an unsubstituted C1-8 heteroalkylene group, a C1-8 heteroalkylene group substituted by 1 to 6 RL1, an unsubstituted 3- to 8-membered arylene group, a 3- to 8-membered arylene group substituted by 1 to 6 RL1, an unsubstituted 3- to 8-membered heteroarylene group with 1 to 3 hetero atoms, and a 3- to 8-membered heteroarylene group with 1 to 3 hetero atoms and substituted by 1 to 6 RL1, an unsubstituted 3- to 7-membered cyclic alkylene group, a 3- to 7-membered cyclic alkylene group substituted by 1 to 6 RL1, an unsubstituted 3- to 7-membered heterocyclic alkylene group with 1 to 2 hetero atoms, and a 3- to 7-membered heterocyclic alkylene group with 1 to 2 hetero atoms and substituted by 1 to 6 RL1; or, X3 is a 6- to 12-membered spiro bicyclic ring having 0-4 heteroatoms;
RL3 is hydrogen;
RL4 and RL5 are independently selected from the group consisting of H, an unsubstituted C1-6 alkyl group, and a C1-6 alkyl group substituted by a C1-6 alkoxyl group or one or more halo groups;
m1 is 0, 1, 2, 3, 4, 5 or 6;
m2 is 0 or 1;
m3 is 0 or 1;
m4 is 1; and
m5 is 0 or 1.

In some embodiment, the heteroatoms in Formula (IV) are each independently selected N, O and S.

In some embodiment, the Z ring comprises one or two heteroatoms selected from N, O and S. In some embodiment, Z is selected from the group consisting of a 3- to 8-membered ring, a 6- to 10-membered bicyclic ring and a 8- to 10-membered spiro bicyclic ring, each independently having 1-2 heteroatoms.

In some embodiment, RL1 is selected from the group consisting of H, an unsubstituted linear C₁₋₃ alkyl group, a keto group, or an oxide group;

In some embodiment, RL2 is a bond or an ethynylene group.

In some embodiment, X1 is selected from the group consisting of a methylene group and an ethylene.

In some embodiment, X2 is selected from the group consisting of C(═O), NRL3C(═O) and O.

In some embodiment, X3 is selected from the group consisting of an unsubstituted C₁₋₆ alkylene group, an unsubstituted 3- to 7-membered cyclic alkylene group, and an unsubstituted 3- to 7-heterocyclic alkylene group with 0 to 2 hetero atoms; or, X3 is a 10- to 12-membered spiro bicyclic ring having 0-2 heteroatoms.

In some embodiment, X4 is selected from the group consisting of a methylene group, CONRL3 and O.

In some embodiment, m1 is 0, 1, 2 or 3.

In some embodiment, RL1 is selected from the group consisting of H, an unsubstituted C1-3 alkyl group, a C1-3 alkyl group substituted by a C1-3 alkoxyl group or one or more halo groups, halogen, a C1-3 alkoxy group, a keto group, or an oxide group; wherein, two RL1 groups are optionally taken together to form a 3-8 membered ring system containing 0-2 heteroatoms. In some embodiment, RL1 is an oxide group which is attached to one heteroatom N on the Z ring to form an N-oxide group (N⁺—O⁻). In some embodiment, RL1 is an alkyl selected from the group consisting of a linear C1-6 alkyl and a branched C1-6 alkyl, each of which is unsubstituted or substituted by a C1-6 alkoxyl group or one or more halo groups. In some embodiment, RL1 is an alkyl selected from the group consisting of a linear C1-3 alkyl and a branched C1-3 alkyl, each of which is unsubstituted or substituted by a C1-6 alkoxyl group or one or more halo groups.

In some embodiment, X3 is selected from the group consisting of a bond, an unsubstituted C1-6 alkylene group, a C1-6 alkylene group substituted by 1 to 6 RL1, an unsubstituted C₁₋₆ heteroalkylene group, a C₁₋₆ heteroalkylene group substituted by 1 to 6 RL1. In some embodiment, X3 is selected from the group consisting of an unsubstituted C1-5 alkylene group.

In some embodiment, RL4 and RL5 are independently an alkyl selected from the group consisting of a linear C₁₋₆ alkyl and a branched C1-6 alkyl, each of which is unsubstituted or substituted by a C1-6 alkoxyl group or one or more halo groups. In some embodiment, RL4 and RL5 are independently an alkyl selected from the group consisting of a linear C1-3 alkyl and a branched C1-3 alkyl, each of which is unsubstituted or substituted by a C1-6 alkoxyl group or one or more halo groups.

In some embodiment, the linker moiety is selected from the group consisting of:

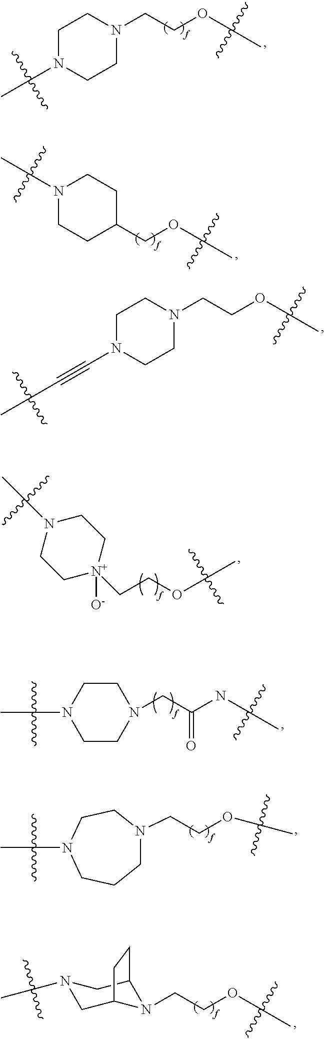

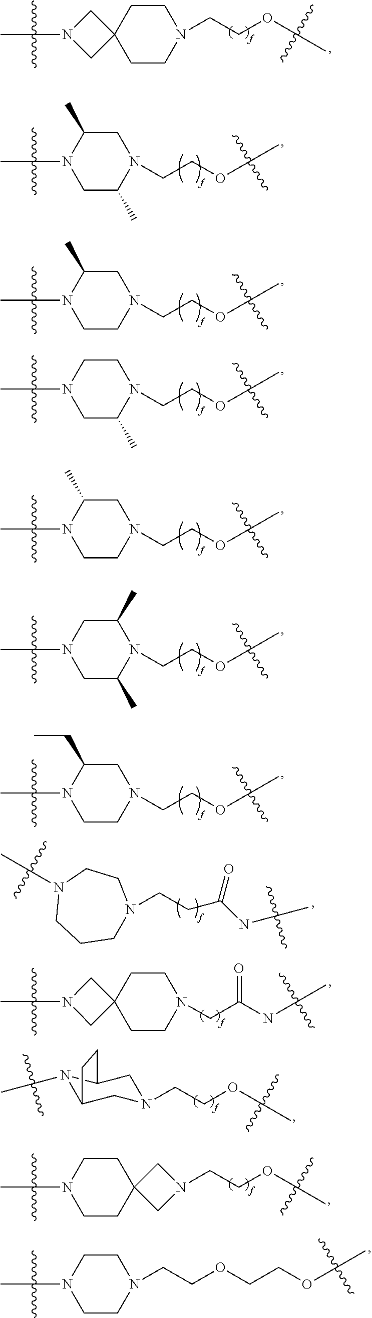

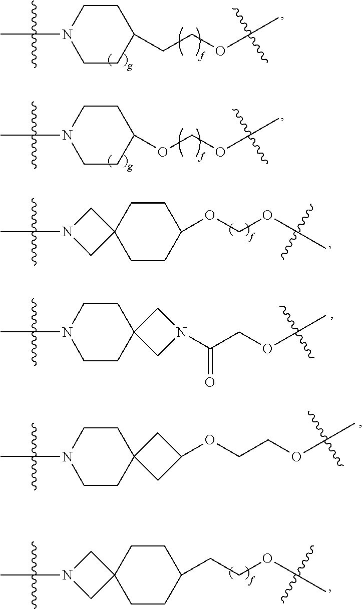

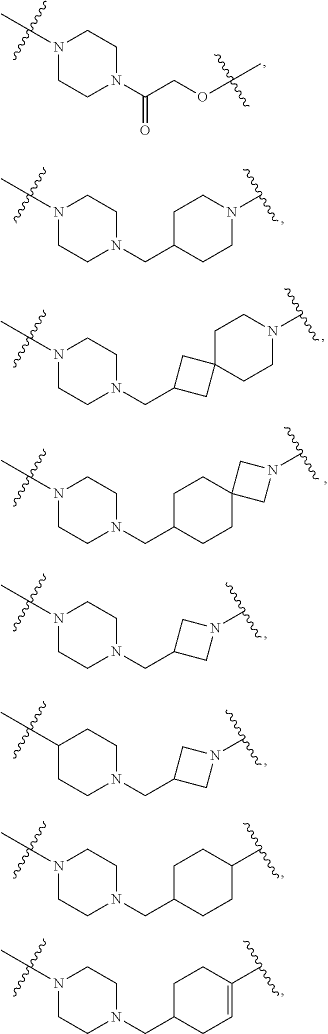

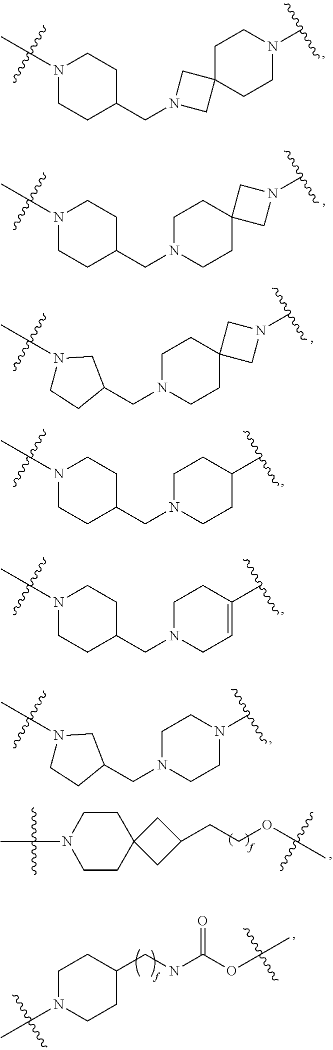

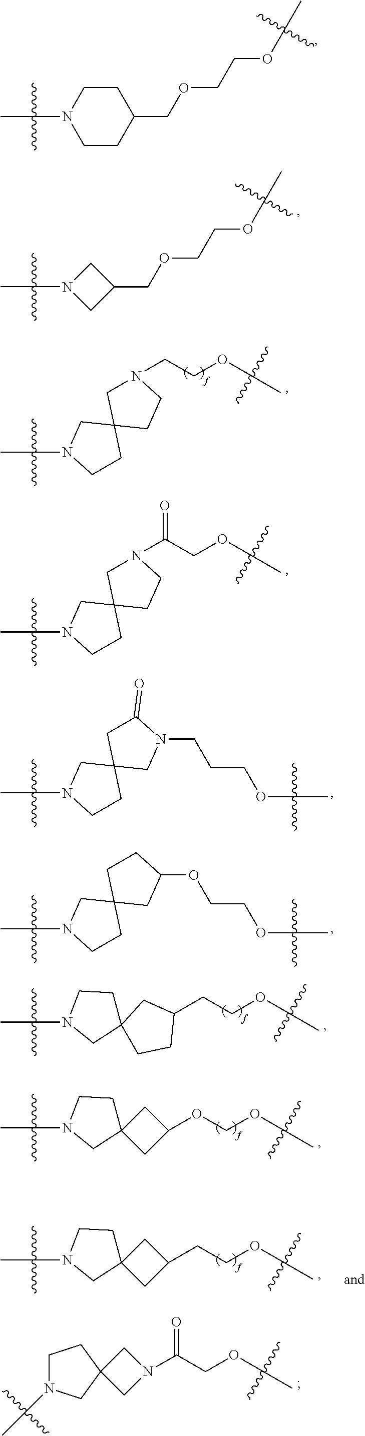

wherein f is an integer of 0, 1, 2, 3 or 4; and g is an integer of 0, 1, 2 or 3.

In some embodiment, the bivalent compound selected from the group consisting of

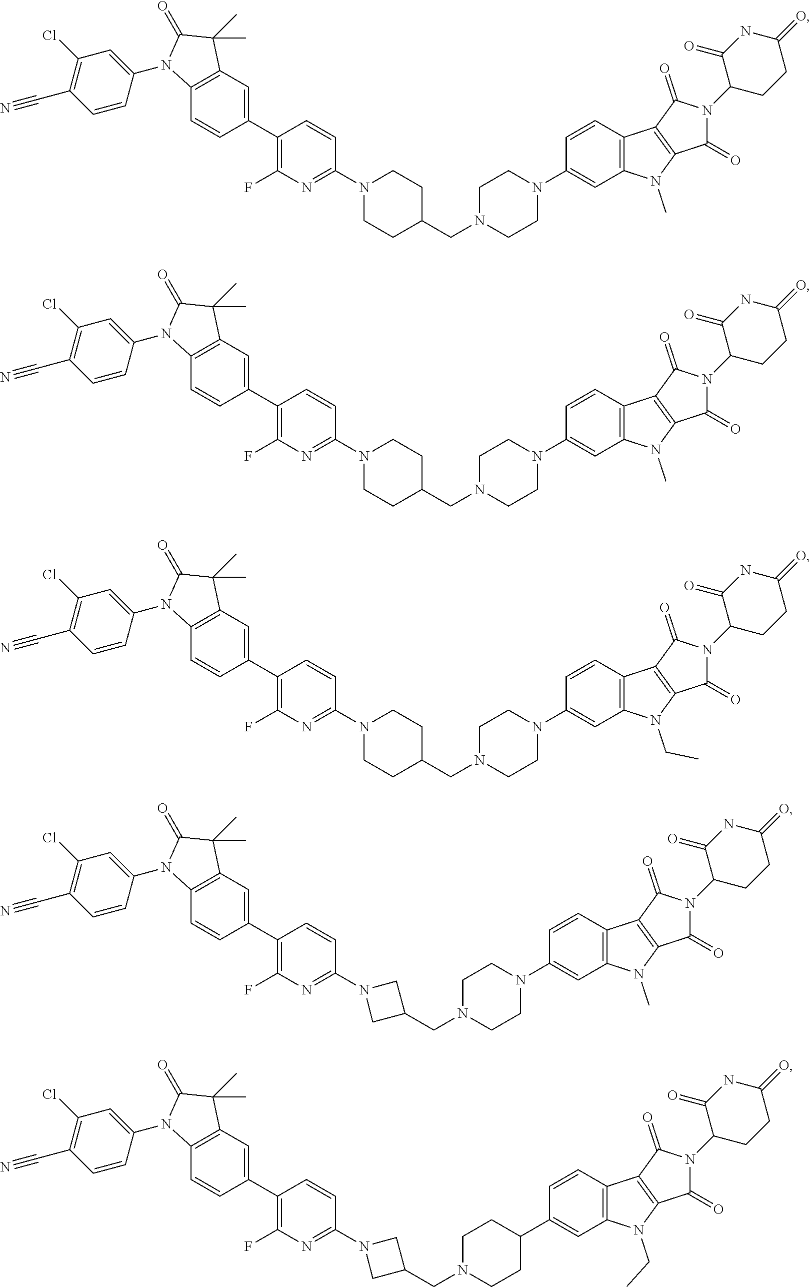

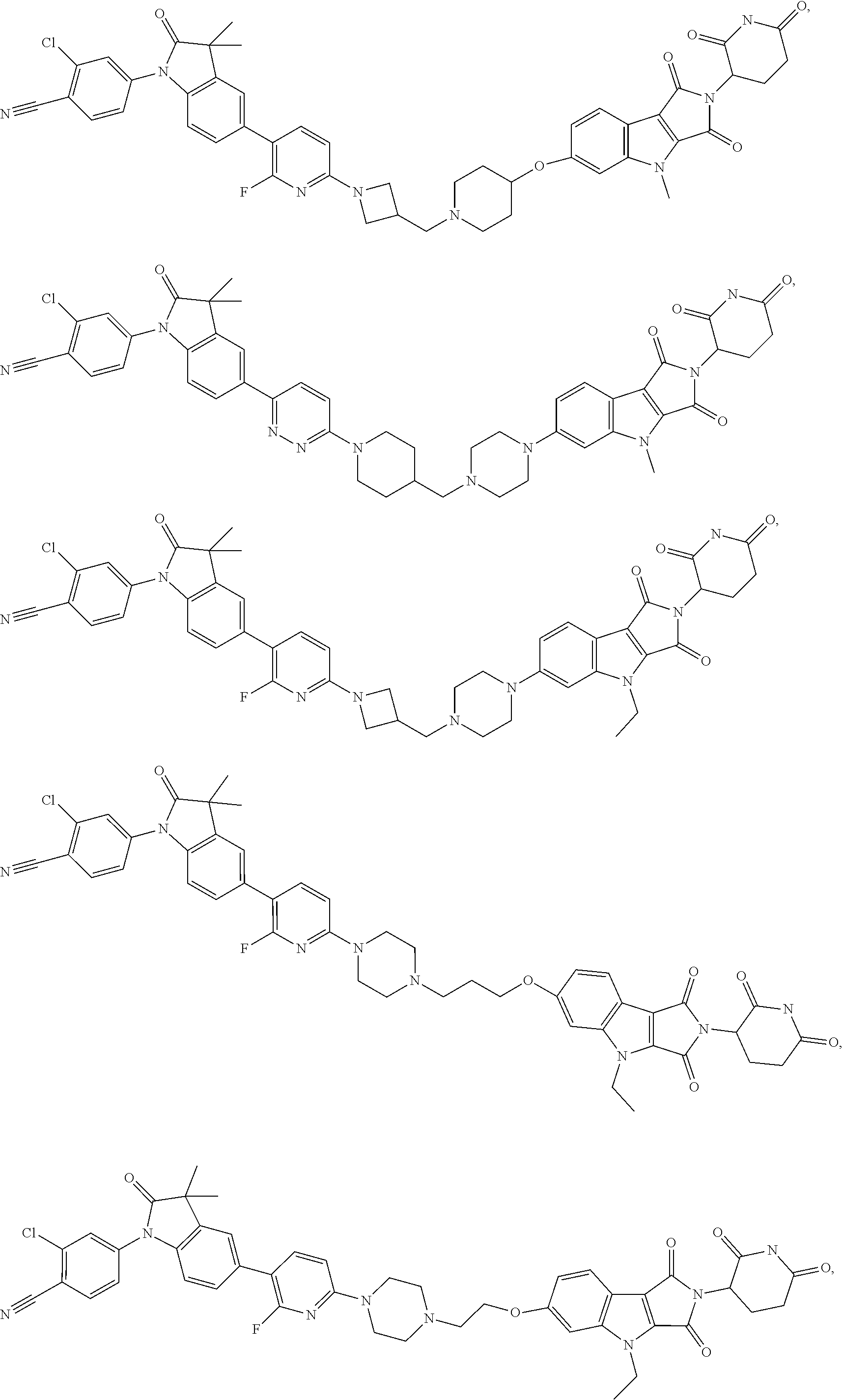

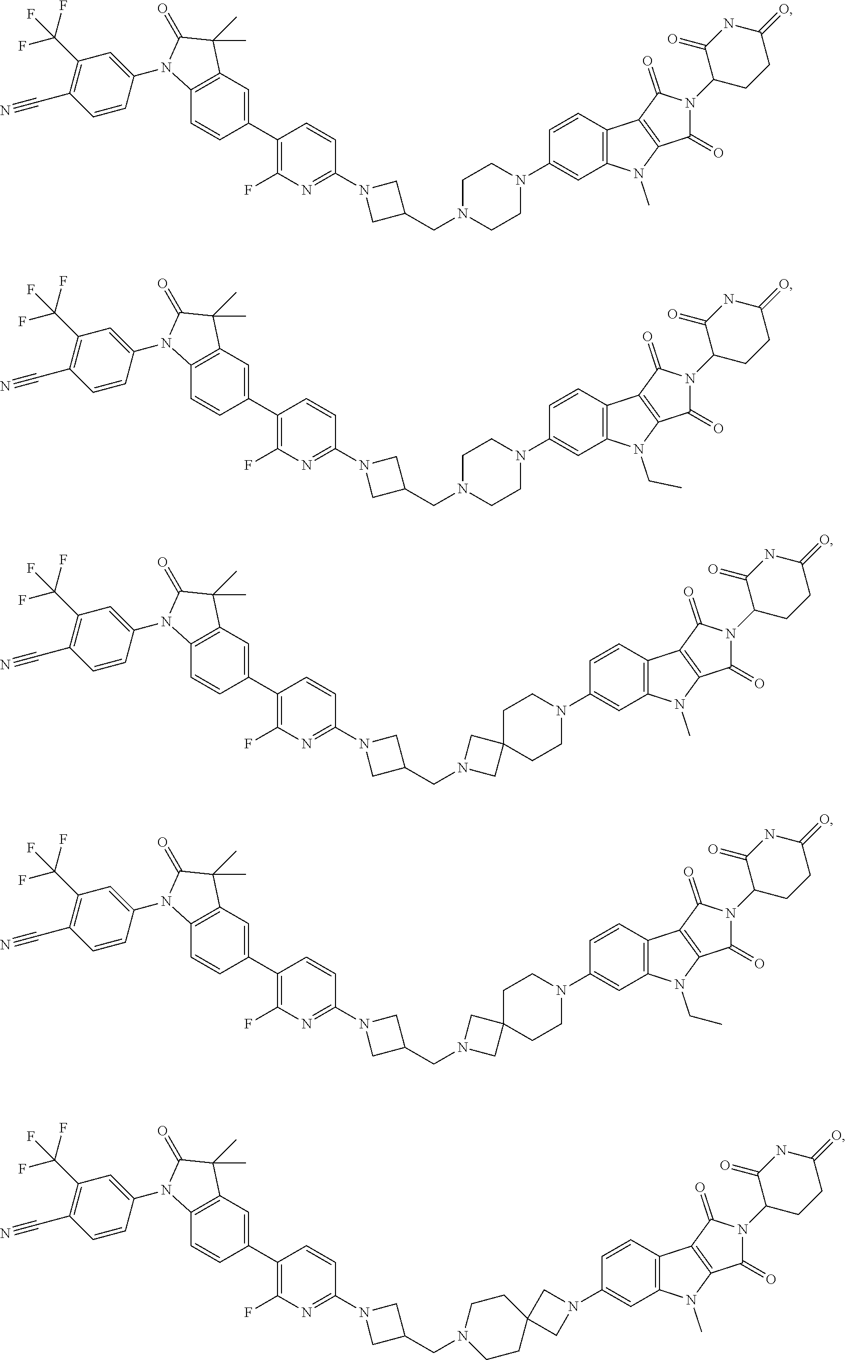

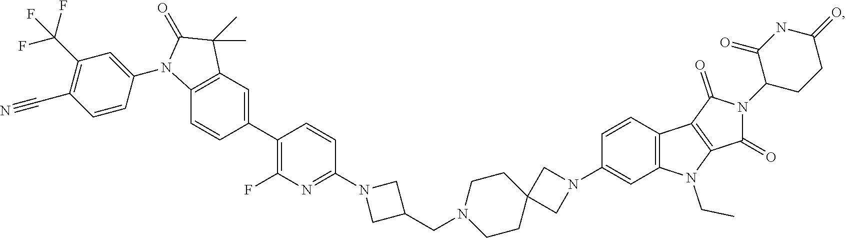 [SMILES: CCn1c2c(c3ccc(N4CC5(CCN(CC6CN(c7ccc(-c8ccc9c(c8)C(C)(C)C(=O)N9c8ccc(C#N)c(C(F)(F)F)c8)c(F)n7)C6)CC5)C4)cc31)C(=O)N(C1CCC(=O)NC1=O)C2=O]

or pharmaceutically acceptable salts thereof.

Another aspect of the present disclosure relates to a composition comprising a bivalent compound disclosed herein, and a pharmaceutically acceptable carrier.

Another aspect of the present disclosure relates to a method of degrading a protein associated with a disease or condition, by contacting the protein with the bivalent compound disclosed herein.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### DETAILED DESCRIPTION OF THE DISCLOSURE

Without wishing to be bound by any theory, the present disclosure is based, at least in part, on the discovery that protein-protein interactions are difficult to be targeted using small molecules because proteins have large contact surfaces and the shallow grooves or flat interfaces thereon may get involved in the interactions. On the other hand, tagging the pathogenic protein with ubiquitin and the eventual degradation by the 26S proteasome system has demonstrated that this modality can provide an extended and thorough removal of the cause of disease (Sun et al., Signal Transduct. Target Ther. 2019, 4:64). In addition to the E1 and E2 enzymes, E3 ubiquitin ligases (also known as E3 ligases) and their substrate recognition proteins confer the substrate specificity for ubiquitination. They are the critical components for specific and selective degradation of target protein substrates. Recent development of targeted protein degradation indicated E3 ligases such as cereblon (CRBN) E3 ligase, von Hippel-Lindau disease tumor suppressor (VHL) E3 ligase, double minute 2 protein (MDM2) E3 ligase, and cell inhibitor of apoptosis protein (cIAP) E3 ligase have been utilized successfully for small molecule protein degrader design.

One E3 ligase with therapeutic potential is cereblon E3 ligase, a protein in humans that is encoded by the CRBN gene. CRBN orthologs are highly conserved from plants to humans. Cereblon forms an E3 ubiquitin ligase complex with damaged DNA binding protein 1 (DDB1), Cullin-4A (CUL4A), and Regulator of Cullins 1 (ROC 1). This complex ubiquitinates a number of other proteins (Vriend et al., Front Mol Biosci. 2018, 5:19).

### Definition of Terms

As used herein in the specification and in the claims, the articles “a” and “an” are used to refer to one or to more than one (i.e., to at least one) of the grammatical object of the article unless the context clearly indicates otherwise. By way of example, “an element” means one element or more than one element.

As used herein in the specification and in the claims, the phrase “and/or,” should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.”

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively.

As used herein in the specification and in the claims, the phrase “at least one,” in reference to a list of one or more elements, should be understood to mean at least one element selected from anyone or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a nonlimiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least 5 one, optionally including more than one, B, with no A present (and optionally including elements other than A); in yet another embodiment, to at least one, optionally including more than one, A, and at least one, optionally including more than one, B (and optionally including other elements); etc.

“Alkyl” refers to a straight or branched hydrocarbon chain radical consisting solely of carbon and hydrogen atoms, containing no unsaturation. An alkyl may comprise one, two, three, four, five, six, seven, eight, nine, ten, eleven, twelve, thirteen, fourteen, fifteen, or sixteen carbon atoms. In certain embodiments, an alkyl comprises one to fifteen carbon atoms (e.g., C₁-C₁₅ alkyl). In certain embodiments, an alkyl comprises one to thirteen carbon atoms (e.g., C₁-C₁₃ alkyl). In certain embodiments, an alkyl comprises one to eight carbon atoms (e.g., C₁-C₈ alkyl). In other embodiments, an alkyl comprises five to fifteen carbon atoms (e.g., C₅-C₁₅ alkyl). In other embodiments, an alkyl comprises five to eight carbon atoms (e.g., C₅-C₈alkyl). The alkyl is attached to the rest of the molecule by a single bond, for example, methyl (Me), ethyl (Et), n-propyl, 1-methylethyl (iso-propyl), n-butyl, n-pentyl, 1,1-dimethylethyl (t-butyl), pentyl, 3-methylhexyl, 2-methylhexyl, and the like.

“Alkenyl” refers to a straight or branched hydrocarbon chain radical group consisting solely of carbon and hydrogen atoms, containing at least one double bond. An alkenyl may comprise two, three, four, five, six, seven, eight, nine, ten, eleven, twelve, thirteen, fourteen, fifteen, or sixteen carbon atoms. In certain embodiments, an alkenyl comprises two to twelve carbon atoms (e.g., C₂-C₁₂ alkenyl). In certain embodiments, an alkenyl comprises two to eight carbon atoms (e.g., C₂-C₈ alkenyl). In certain embodiments, an alkenyl comprises two to six carbon atoms (e.g., C₂-C₆ alkenyl). In other embodiments, an alkenyl comprises two to four carbon atoms (e.g., C₂-C₄ alkenyl). The alkenyl is attached to the rest of the molecule by a single bond, for example, ethenyl (i.e., vinyl), prop-1-enyl (i.e., allyl), but-1-enyl, pent-1-enyl, penta-1,4-dienyl, and the like.

The term “allyl,” as used herein, means a —CH₂CH═CH₂ group.

As used herein, the term “alkynyl” refers to a straight or branched hydrocarbon chain radical group consisting solely of carbon and hydrogen atoms, containing at least one triple bond. An alkynyl may comprise two, three, four, five, six, seven, eight, nine, ten, eleven, twelve, thirteen, fourteen, fifteen, or sixteen carbon atoms. In certain embodiments, an alkynyl comprises two to twelve carbon atoms (e.g., C₂-C₁₂ alkynyl). In certain embodiments, an alkynyl comprises two to eight carbon atoms (e.g., C₂-C₈ alkynyl). In other embodiments, an alkynyl has two to six carbon atoms (e.g., C₂-C₆ alkynyl). In other embodiments, an alkynyl has two to four carbon atoms (e.g., C₂-C₄ alkynyl). The alkynyl is attached to the rest of the molecule by a single bond. Examples of such groups include, but are not limited to, ethynyl, propynyl, 1-butynyl, 2-butynyl, 1-pentynyl, 2-pentynyl, 1-hexynyl, 2-hexynyl, 3-hexynyl, and the like.

The term “alkoxy”, as used herein, means an alkyl group as defined herein which is attached to the rest of the molecule via an oxygen atom. Examples of such groups include, but are not limited to, methoxy, ethoxy, n-propyloxy, iso-propyloxy, n-butoxy, iso-butoxy, tert-butoxy, pentyloxy, hexyloxy, and the like.

The term “aryl”, as used herein, refers to a radical derived from an aromatic monocyclic or multicyclic hydrocarbon ring system by removing a hydrogen atom from a ring carbon atom. The aromatic monocyclic or multicyclic hydrocarbon ring system contains only hydrogen and carbon atoms. An aryl may comprise from six to eighteen carbon atoms, where at least one of the rings in the ring system is fully unsaturated, i.e., it contains a cyclic, delocalized (4n+2) π-electron system in accordance with the Hückel theory. In certain embodiments, an aryl comprises six to fourteen carbon atoms (C₆-C₁₄ aryl). In certain embodiments, an aryl comprises six to ten carbon atoms (C₆-C₁₀ aryl). Examples of such groups include, but are not limited to, phenyl, fluorenyl and naphthyl. The terms “Ph” and “phenyl,” as used herein, mean a —C₆H₅ group.

The term “heteroaryl”, refers to a radical derived from a 3- to 18-membered aromatic ring radical that comprises two to seventeen carbon atoms and from one to six heteroatoms selected from nitrogen, oxygen and sulfur. As used herein, the heteroaryl radical may be a monocyclic, bicyclic, tricyclic or tetracyclic ring system, wherein at least one of the rings in the ring system is fully unsaturated, i.e., it contains a cyclic, delocalized (4n+2) π-electron system in accordance with the Hückel theory. Heteroaryl includes fused or bridged ring systems. The heteroatom(s) in the heteroaryl radical is optionally oxidized. One or more nitrogen atoms, if present, are optionally quaternized. The heteroaryl is attached to the rest of the molecule through any atom of the ring(s). Examples of such groups include, but not limited to, pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, thiadiazolyl, furazanyl, benzofurazanyl, benzothiophenyl, benzothiazolyl, benzoxazolyl, quinazolinyl, quinoxalinyl, naphthyridinyl, furopyridinyl, and the like. In certain embodiments, an heteroaryl is attached to the rest of the molecule via a ring carbon atom. In certain embodiments, an heteroaryl is attached to the rest of the molecule via a nitrogen atom (N-attached) or a carbon atom (C-attached). For instance, a group derived from pyrrole may be pyrrol-1-yl (N-attached) or pyrrol-3-yl (C-attached). Further, a group derived from imidazole may be imidazol-1-yl (N-attached) or imidazol-3-yl (C-attached).

The term “heterocyclyl”, as used herein, means a non-aromatic, monocyclic, bicyclic, tricyclic, or tetracyclic radical having a total of from 4, 5, 6, 7, 8, 9, 10, 11, 12, or 13 atoms in its ring system, and containing from 3 to 12 carbon atoms and from 1 to 4 heteroatoms each independently selected from O, S and N, and with the proviso that the ring of said group does not contain two adjacent O atoms or two adjacent S atoms. A heterocyclyl group may include fused, bridged or spirocyclic ring systems. In certain embodiments, a hetercyclyl group comprises 3 to 10 ring atoms (3-10 membered heterocyclyl). In certain embodiments, a hetercyclyl group comprises 3 to 8 ring atoms (3-8 membered heterocyclyl). In certain embodiments, a hetercyclyl group comprises 4 to 8 ring atoms (4-8 membered heterocyclyl). In certain embodiments, a hetercyclyl group comprises 3 to 6 ring atoms (3-6 membered heterocyclyl). A heterocyclyl group may contain an oxo substituent at any available atom that will result in a stable compound. For example, such a group may contain an oxo atom at an available carbon or nitrogen atom. Such a group may contain more than one oxo substituent if chemically feasible. In addition, it is to be understood that when such a heterocyclyl group contains a sulfur atom, said sulfur atom may be oxidized with one or two oxygen atoms to afford either a sulfoxide or sulfone. An example of a 4 membered heterocyclyl group is azetidinyl (derived from azetidine). An example of a 5 membered cycloheteroalkyl group is pyrrolidinyl. An example of a 6 membered cycloheteroalkyl group is piperidinyl. An example of a 9 membered cycloheteroalkyl group is indolinyl. An example of a 10 membered cycloheteroalkyl group is 4H-quinolizinyl. Further examples of such heterocyclyl groups include, but are not limited to, tetrahydrofuranyl, dihydrofuranyl, tetrahydrothienyl, tetrahydropyranyl, dihydropyranyl, tetrahydrothiopyranyl, piperidino, morpholino, thiomorpholino, thioxanyl, piperazinyl, azetidinyl, oxetanyl, thietanyl, homopiperidinyl, oxepanyl, thiepanyl, oxazepinyl, diazepinyl, thiazepinyl, 1,2,3,6-tetrahydropyridinyl, 2-pyrrolinyl, 3-pyrrolinyl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, pyrazolidinyl, imidazolinyl, imidazolidinyl, 3-azabicyclo[3.1.0]hexanyl, 3-azabicyclo[4.1.0]heptanyl, 3H-indolyl, quinolizinyl, 3-oxopiperazinyl, 4-methylpiperazinyl, 4-ethylpiperazinyl, and 1-oxo-2,8,diazaspiro[4.5]dec-8-yl. A heteroaryl group may be attached to the rest of molecular via a carbon atom (C-attached) or a nitrogen atom (N-attached). For instance, a group derived from piperazine may be piperazin-1-yl (N-attached) or piperazin-2-yl (C-attached).

The term “cycloalkyl” means a saturated, monocyclic, bicyclic, tricyclic, or tetracyclic radical having a total of from 4, 5, 6, 7, 8, 9, 10, 11, 12, or 13 carbon atoms in its ring system. A cycloalkyl may be fused, bridged or spirocyclic. In certain embodiments, a cycloalkyl comprises 3 to 8 carbon ring atoms (C₃-C₈ cycloalkyl). In certain embodiments, a cycloalkyl comprises 3 to 6 carbon ring atoms (C₃-C₆ cycloalkyl). Examples of such groups include, but are not limited to, cyclopropyl, cyclobutyl, cyclopentyl, cyclopentenyl, cyclohexyl, cycloheptyl, adamantyl, and the like.

The term “cycloalkylene” is a bidentate radical obtained by removing a hydrogen atom from a cycloalkyl ring as defined above. Examples of such groups include, but are not limited to, cyclopropylene, cyclobutylene, cyclopentylene, cyclopentenylene, cyclohexylene, cycloheptylene, and the like.

The term “spirocyclic” as used herein has its conventional meaning, that is, any ring system containing two or more rings wherein two of the rings have one ring carbon in common. Each ring of the spirocyclic ring system, as herein defined, independently comprises 3 to 20 ring atoms. Preferably, they have 3 to 10 ring atoms. Non-limiting examples of a spirocyclic system include spiro[3.3]heptane, spiro[3.4]octane, and spiro[4.5]decane.

The term “cyano” refers to a —C≡N group.

An “aldehyde” group refers to a —C(O)H group.

An “alkoxy” group refers to both an —O-alkyl, as defined herein.

An “alkoxycarbonyl” refers to a —C(O)-alkoxy, as defined herein.

An “alkylaminoalkyl” group refers to an -alkyl-NR-alkyl group, as defined herein.

An “alkylsulfonyl” group refer to a —SO₂alkyl, as defined herein.

An “amino” group refers to an optionally substituted —NH₂.

An “aminoalkyl” group refers to an -alky-amino group, as defined herein.

An “aminocarbonyl” refers to a —C(O)-amino, as defined herein.

An “arylalkyl” group refers to -alkylaryl, where alkyl and aryl are defined herein.

An “aryloxy” group refers to both an —O-aryl and an —O-heteroaryl group, as defined herein.

An “aryloxycarbonyl” refers to —C(O)-aryloxy, as defined herein.

An “arylsulfonyl” group refers to a —SO₂aryl, as defined herein.

A “carbonyl” group refers to a —C(O)— group, as defined herein.

A “carboxylic acid” group refers to a —C(O)OH group.

A “cycloalkoxy” refers to a —O-cycloalkyl group, as defined herein.

A “halo” or “halogen” group refers to fluorine, chlorine, bromine or iodine.

A “haloalkyl” group refers to an alkyl group substituted with one or more halogen atoms.

A “hydroxy” group refers to an —OH group.

A “nitro” group refers to a —NO₂ group.

An “oxo” group refers to the ═O substituent.

A “trihalomethyl” group refers to a methyl substituted with three halogen atoms.

The term “substituted,” means that the specified group or moiety bears one or more substituents independently selected from C₁-C₄ alkyl, aryl, heteroaryl, aryl-C₁-C₄ alkyl-, heteroaryl-C₁-C₄ alkyl-, C₁-C₄ haloalkyl, —OC₁-C₄ alkyl, —OC₁-C₄ alkylphenyl, —C₁-C₄ alkyl-OH, —OC₁-C₄ haloalkyl, halo, —OH, —NH₂, —C₁-C₄ alkyl-NH₂, —N(C₁-C₄ alkyl)(C₁-C₄ alkyl), —NH(C₁-C₄ alkyl), —N(C₁-C₄ alkyl)(C₁-C₄ alkylphenyl), —NH(C₁-C₄ alkylphenyl), cyano, nitro, oxo, —CO₂H, —C(O)OC₁-C₄ alkyl, —CON(C₁-C₄ alkyl)(C₁-C₄ alkyl), —CONH(C₁-C₄ alkyl), —CONH₂, —NHC(O)(C₁-C₄ alkyl), —NHC(O)(phenyl), —N(C₁-C₄ alkyl)C(O)(C₁-C₄ alkyl), —N(C₁-C₄ alkyl)C(O)(phenyl), —C(O)C₁-C₄ alkyl, —C(O)C₁-C₄ alkylphenyl, —C(O)C₁-C₄ haloalkyl, —OC(O)C₁-C₄ alkyl, —SO₂(C₁-C₄ alkyl), —SO₂(phenyl), —SO₂(C₁-C₄ haloalkyl), —SO₂NH₂, —SO₂NH(C₁-C₄ alkyl), —SO₂NH(phenyl), —NHSO₂(C₁-C₄ alkyl), —NHSO₂(phenyl), and —NHSO₂(C₁-C₄ haloalkyl).

The term “optionally substituted” means that the specified group may be either unsubstituted or substituted by one or more substituents as defined herein. It is to be understood that in the compounds of the present invention when a group is said to be “unsubstituted,” or is “substituted” with fewer groups than would fill the valencies of all the atoms in the compound, the remaining valencies on such a group are filled by hydrogen. For example, if a C₆ aryl group, also called “phenyl” herein, is substituted with one additional substituent, one of ordinary skill in the art would understand that such a group has 4 open positions left on carbon atoms of the C₆ aryl ring (6 initial positions, minus one at which the remainder of the compound of the present invention is attached to and an additional substituent, remaining 4 positions open). In such cases, the remaining 4 carbon atoms are each bound to one hydrogen atom to fill their valencies. Similarly, if a C₆ aryl group in the present compounds is said to be “disubstituted,” one of ordinary skill in the art would understand it to mean that the C₆ aryl has 3 carbon atoms remaining that are unsubstituted. Those three unsubstituted carbon atoms are each bound to one hydrogen atom to fill their valencies.

“Pharmaceutically acceptable salt” includes both acid and base addition salts. A pharmaceutically acceptable salt of any one of the bivalent compounds described herein is intended to encompass any and all pharmaceutically suitable salt forms. Preferred pharmaceutically acceptable salts of the compounds described herein are pharmaceutically acceptable acid addition salts and pharmaceutically acceptable base addition salts.

“Pharmaceutically acceptable acid addition salt” refers to those salts which retain the biological effectiveness and properties of the free bases, which are not biologically or otherwise undesirable, and which are formed with inorganic acids such as hydrochloric acid, hydrobromic acid, sulfuric acid, nitric acid, phosphoric acid, hydroiodic acid, hydrofluoric acid, phosphorous acid, and the like. Also included are salts that are formed with organic acids such as aliphatic mono- and dicarboxylic acids, phenyl-substituted alkanoic acids, hydroxy alkanoic acids, alkanedioic acids, aromatic acids, aliphatic and. aromatic sulfonic acids, etc. and include, for example, acetic acid, trifluoroacetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Exemplary salts thus include sulfates, pyrosulfates, bisulfates, sulfites, bisulfites, nitrates, phosphates, monohydrogenphosphates, dihydrogenphosphates, metaphosphates, pyrophosphates, chlorides, bromides, iodides, acetates, trifluoroacetates, propionates, caprylates, isobutyrates, oxalates, malonates, succinate suberates, sebacates, fumarates, maleates, mandelates, benzoates, chlorobenzoates, methylbenzoates, dinitrobenzoates, phthalates, benzenesulfonates, toluenesulfonates, phenylacetates, citrates, lactates, malates, tartrates, methanesulfonates, and the like. Also contemplated are salts of amino acids, such as arginates, gluconates, and galacturonates (see, for example, Berge S. M. et al., “Pharmaceutical Salts,” Journal of Pharmaceutical Science, 66:1-19 (1997), which is hereby incorporated by reference in its entirety). Acid addition salts of basic compounds may be prepared by contacting the free base forms with a sufficient amount of the desired acid to produce the salt according to methods and techniques with which a skilled artisan is familiar.

“Pharmaceutically acceptable base addition salt” refers to those salts that retain the biological effectiveness and properties of the free acids, which are not biologically or otherwise undesirable. These salts are prepared from addition of an inorganic base or an organic base to the free acid. Pharmaceutically acceptable base addition salts may be formed with metals or amines, such as alkali and alkaline earth metals or organic amines. Salts derived from inorganic bases include, but are not limited to, sodium, potassium, lithium, ammonium, calcium, magnesium, iron, zinc, copper, manganese, aluminum salts and the like. Salts derived from organic bases include, but are not limited to, salts of primary, secondary, and tertiary amines, substituted amines including naturally occurring substituted amines, cyclic amines and basic ion exchange resins, for example, isopropylamine, trimethylamine, diethylamine, triethylamine, tripropylamine, ethanolamine, diethanolamine, 2-dimethylaminoethanol, 2-diethylaminoethanol, dicyclohexylamine, lysine, arginine, histidine, caffeine, procaine, N,N-dibenzylethylenediamine, chloroprocaine, hydrabamine, choline, betaine, ethylenediamine, ethylenedianiline, N-methylglucamine, glucosamine, methylglucamine, theobromine, purines, piperazine, piperidine, N-ethylpiperidine, polyamine resins and the like. See Berge et al., supra.

### Bivalent Compound

In some aspects, the present disclosure provides a bivalent compound including a target binding moiety (TBM) and a cereblon E3 ubiquitin ligase binding moiety (CLM), or a pharmaceutically acceptable salt or analog thereof. The TBM may be connected to the CLM directly or via a linker moiety. In certain embodiments, the TRK ligand may be connected to the degradation tag directly (TBM-CLM). In certain embodiments, the TRK ligand may be connected to the degradation tag via a linker moiety (TBM-L-CLM).

### Target Binding Moiety (TBM)

As used herein, the terms “target binding moiety” or “TBM”, may refer to any molecules ranging from small molecules to large proteins that associate with or bind to a target protein to be degraded by the bivalent compounds disclosed herein, i.e. protein of interest (“POI”). The TBM can be, for example but not limited to, a small molecule compound (i.e., a molecule of molecular weight less than about 1.5 kilodaltons (kDa)), a peptide or polypeptide, nucleic acid or oligonucleotide, carbohydrate such as oligosaccharides, or an antibody or fragment thereof. In certain embodiments, the TBM is capable of binding to an overexpressed protein associated with a disease or condition, a mutated protein associated with a disease or condition, a fusion protein associated with a disease or condition, or the like.

### Targets Suitable for Degradation

As described above, the first wave of clinical-stage protein degraders is aimed at classically drugged targets that have clinically validated roles in disease and readily available chemical matter. Success against these targets has begun to solidify PROTACs as a therapeutic modality and underscores the potential of these molecules to become best-in-class medicines by way of degrading a target instead of inhibiting it. However, the true promise of the modality is reaching targets that are currently difficult to drug with existing modalities or have not yet been drugged at all.

To date, traditional small-molecule drug discovery research for intracellular targets has often focused on developing high-affinity inhibitors that target either the active site or an allosteric site on an enzyme to shut down the function of the POI (occupancy-driven pharmacology). Although this has been a highly effective approach, it has left potential drug targets undrugged or underdrugged. PROTACs bring the degradation function to the target (event-driven pharmacology), negating the need for an active site and redefining undruggable targets as simply undrugged.

The targets for PROTAC therapy can have several common characteristics, including: a change away from the natural state, via overexpression, mutation, aggregation, isoform expression or localization, that results in a disease-causing gain of function; a binding surface that is approachable by an E3 ligase; and an unstructured region to thread into the proteasome. Proteins that have evolved resistance mutations to targeted therapies, proteins with scaffolding functions and proteins that are considered ‘undruggable’ with other modalities can also be highly suitable PROTAC targets. The early PROTAC targets focused on POIs that had existing ligands, in the form of available inhibitors, but were still associated with clear unmet medical need. There are several reasons that existing ligands failed to fully benefit patients, such as incomplete inhibition, narrow therapeutic window and partial selectivity. By incorporating the ligand into a PROTAC, which often acts in an iterative or ‘pseudo-catalytic’ fashion by binding and facilitating the interaction of its ligase and POI targets, the POI can be degraded without requiring a large excess of drug, thereby resulting in more complete target blockade and a wider therapeutic window. PROTACs may also benefit from cooperative PPIs between the E3 ligase and the POI. This can have the advantages of improved potency and selectivity.

Although ‘PROTACable’ POIs do not necessarily need an enzyme active site, they do need a small-molecule binding site that is approachable by an E3 ligase. Using these sites does not require a high-affinity ligand if coupled to the right E3 ligand, but moderate affinity (≥1-500 nM) is typically needed, and access to the POI surface near the binding site by a recruited E3 ligase is essential. Achieving such binding affinities can often be challenging and has promoted research into alternative degraders. Selection of the ligand-binding site is particularly important in the case of scaffolding proteins, where the POI may only be partially exposed within a given complex. It may be possible to degrade a POI by targeting a neighboring protein within a protein complex (the bystander effect). This approach may prove useful in degrading scaffolding proteins in which the surface of the POI is mostly buried within the complex or the POI is a membrane-associated protein.

### Examples of POIs

Androgen receptor (AR), a member of the nuclear hormone receptor family, is a transcriptional factor. Upon activation by androgen, AR translocate to the nucleus where it regulates nuclear gene transcription. AR is related to prostate cancer formation and progression, which is the second most prevalent cancer and the second-leading cause of cancer death in men. AR is a clinically fully validated target for the treatment of human prostate cancer, inhibition of AR has been extensively investigated to treat prostate cancer. Because AR protein plays a key role in metastatic castration-resistant prostate cancer (mCRPC), AR degraders designed based upon the concept of PROTAC could be potentially very effective for the treatment of mCRPC when the disease becomes resistant to AR antagonists or to androgen synthesis inhibitors.

Approximately 70% of breast cancer cases are estrogen receptor-alpha-positive (ERα+). The binding of estradiol to the ligand-binding domain activates ERα. ERα can also be activated via the phosphorylation induced by growth factors. Activated ERα functions as a transcriptional regulator with a pro-tumor activity in breast cancer cells. ER degraders designed based upon the concept of PROTAC could be potentially very effective for the treatment of ERα+ breast cancer.

BRD4 is a critical protein that is overexpressed in human cancer and promotes the growth and survival of cancer cells (Donati et al., 2018; Zhang F. et al., 2020). The present disclosure recognizes that development of the first CRBN-based PROTAC in 2015, with the structure of pomalidomide capturing CRBN and BRDs inhibitor JQ1 as TBM. The resulting compound has been shown to induce highly selective CRBN-dependent BET protein degradation in vitro and in vivo and delay the progression of leukemia in mice, demonstrating the high efficiency and specificity of degradation of BRD family members, such as BRD2, BRD3, and BRD4, by using large-scale proteomic methods (Winter et al., 2015).

TGF-β1 is a pleiotropic cytokine and plays an important role in tumor progression (e.g., colorectal and prostate cancer). Also, it is one of the key factors of tumor cell immune escape (Sun D.-Y. et al., 2019; Dai et al., 2019). Feng's team has developed a CRBN-based PROTAC DT-6 to degrade TGF-β1. The TGF-β1 ligand is derived from its direct inhibitor P144, and CRBN is recruited by the widely used ligand thalidomide. It has been shown that DT-6 can effectively degrade TGF-β1 in cells and reduce its secretion, which is of great significance for diseases that are correlated with the TGF-β1 signaling (Feng et al., 2020).

In light of the large effect of structure on degradation efficacy, Su's team has designed a series of PROTACs with varying CDK6 targeting ligands, E3 ligases, and linkers. Considering that the terminal ligands of E3 ligase can also deeply affect the interaction angle between the target protein and the ligase, they have introduced flexible and rigid groups such as alkyl and alkyne into the ligand pomalidomide. To predict which ligase matches CDK6, they have also designed nutlin-3b, VH032, and Bestatin to recruit the E3 ligases MDM2, VHL, and cIAP, respectively. Three FDA-approved CDK4/6 inhibitors (palbociclib, ribociclib, and abemaciclib) have been selected as the binding ligands of the target protein CDK6, which have a strong binding ability to CDK6 with different terminal directions. Finally, it has been found that only CRBN-based PROTAC can degrade CDK6. PROTACs with shorter linkers have shown a higher capacity in CDK6 degradation, suggesting that these shorter molecules have better CRBN recruitment ability on CDK6 (Su et al., 2019).

There are many PROTACs that have been designed with pomalidomide as the CRBN ligand to degrade various POIs, such as MCL-1/BCL-2, BCL-xL, HDAC6, and BTK (Myeku et al., 2016; Sun et al., 2018; Wang X. et al., 2019; Chi et al., 2019; Yang et al., 2019; Xue et al., 2020). Protein-protein interaction (PPI) is involved in most cell processes, including cell differentiation, apoptosis, signal transduction, and transcription (Ryan and Matthews, 2005). Therefore, the role of PPI should not be underestimated, and it has been believed that the target of PPI is the next breakthrough point in disease treatment. Ye's team has used two different BCL-2/MCL-1 inhibitors S1-6 and Nap-1 to develop two different series of PROTACs, C3 and C5 (Wang X. et al., 2019). These PROTACs have shown strong ability in PPI target degradation with DC50 (The 50% of maximum degradation) of 0.7 and 3.0 μM, respectively. This study has verified that PROTACs can extend the “target space” to the PPI target. It provides a selective chemical intervention for BCL-2 family protein in chemical biology research and drug discovery.

BTK, a non-receptor cytoplasmic tyrosine kinase, is involved in B cell receptor (BCR) signaling pathway and plays a key role in B cell lymphoma, so its degradation is particularly important (Hendriks et al., 2014). There are many reports on the degradation of BTK by PROTAC. Using CRBN as the E3 ligase, Crews's team has found that MT802 can effectively degrade BTK. It has excellent degradation characteristics in vitro but shows a high clearance rate and short half-life in vivo. They have further replaced the CRBN ligand with the VHL ligand. Unfortunately, the resulting compound have shown low degradation efficiency. Finally, the structure modification of the CRBN ligand has led to the identification of SJF620, with improved druggability compared with MT802 (Jaime-Figueroa et al., 2020).

Multiple E3 ubiquitin ligases have been selected to degrade the target proteins. Ibrutinib and PLS-123, two covalent inhibitors of BTK, have been chosen as the binding part of BTK due to the high affinity and different folding structures. CRBN and VHL have been selected as the E3 ligase, which were recruited by pomalidomide and VH032, respectively. Once irreversibly combined with target kinase, an excellent degradation efficiency has been observed in living cells (Xue et al., 2020). Different from Pan's team, CRBN and MDM2 have been selected as the E3 ligases in Rao's study (Sun et al., 2018; Xue et al., 2020). In addition to the recruitment of CRBN by pomalidomide, RG-7112 has been designed as the ligand for MDM2 recruitment and ibrutinib and spebrutinib have been selected as the BTK ligands. It has been found that CRBN is generally more effective as E3 ligase than MDM2 (Sun et al., 2018). Besides BTK, CRBN- and VHL-PROTAC can also effectively degrade EGFR, BRD4, PLK1, and CDK2 (Zhou F. et al., 2020; Zhang H. et al., 2020; Mu et al., 2020).

In addition, Li et al. have developed a PROTAC that can degrade the cell cycle kinase Wee1 and provided a new direction for targeted cancer therapy (Jaeger and Winter, 2020; Li et al., 2020). Winzker et al. have described that PDE6-based PROTACs can effectively and selectively reduce the level of phosphodiesterase-6 (PDE6) in cells (Winzker et al., 2020). At the same time, it has also increased the expression of various lipid-related enzymes and the level of cholesterol precursor. The results have also shown that PDE6 plays a role in the regulation of sterol synthesis (Winzker et al., 2020). Signal transducer and activator of transcription 3 (STAT3) activation is beneficial to the survival, reproduction, metastasis, and immune escape of tumor cells (Furtek et al., 2016). STAT3 is closely related to the adverse prognosis of human cancer and has become a promising therapeutic target for cancer and other diseases. Zhou et al. have developed SD-36 as a highly selective and potent PROTAC degrader of STAT3. SD-36 can inhibit the growth of leukemia and lymphoma cell lines with highly phosphorylated STAT3 at low nanomolar concentrations in vitro. SD-36 can also completely and persistently regress the tumor growth in mice bearing the Molm-16 xenografts. SD-36 has been found to rapidly induce the degradation of STAT3 but has no significant effect on other STAT isoforms (Zhou et al., 2019).

Bromodomain and Extra-Terminal domain (BET) family proteins are epigenetic regulatory factors related to the expression of multiple oncogenes (Stathis and Bertoni, 2018). BETd-260 is an effective PROTAC degradation agent synthesized on the basis of BET SMIs. The in vivo and in vitro experiments have shown that it can induce a large amount of apoptosis in osteosarcoma (OS) cells and OS xenograft tumor tissues and ultimately lead to the depth and sustained inhibition of tumor growth in both mouse OS cell line-derived xenograft and patient-derived xenograft (PDX) models (Shi et al., 2019).

In some embodiments, the TBM is an antagonist of the POI. In some embodiments, the TBM is an agonist of the POI. In some embodiments, the TBM is a partial agonist of the POI. In some embodiments, the TBM is an inverse agonist of the POI.

In some embodiments, the TBM is not selected from the group consisting of:

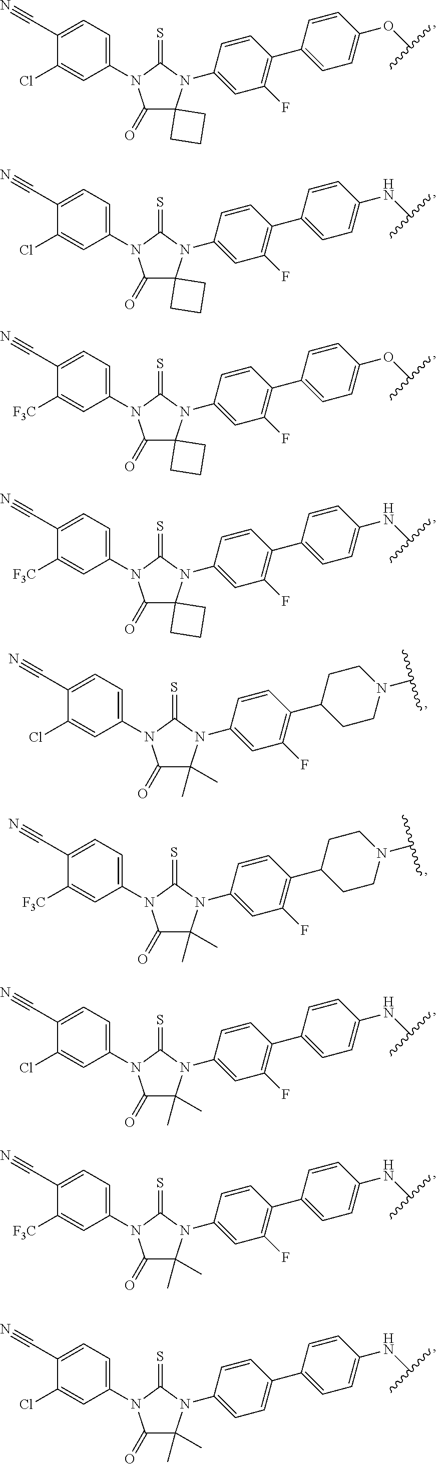

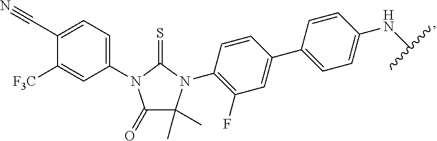 [SMILES: *Nc1ccc(-c2ccc(N3C(=S)N(c4ccc(C#N)c(C(F)(F)F)c4)C(=O)C3(C)C)c(F)c2)cc1]

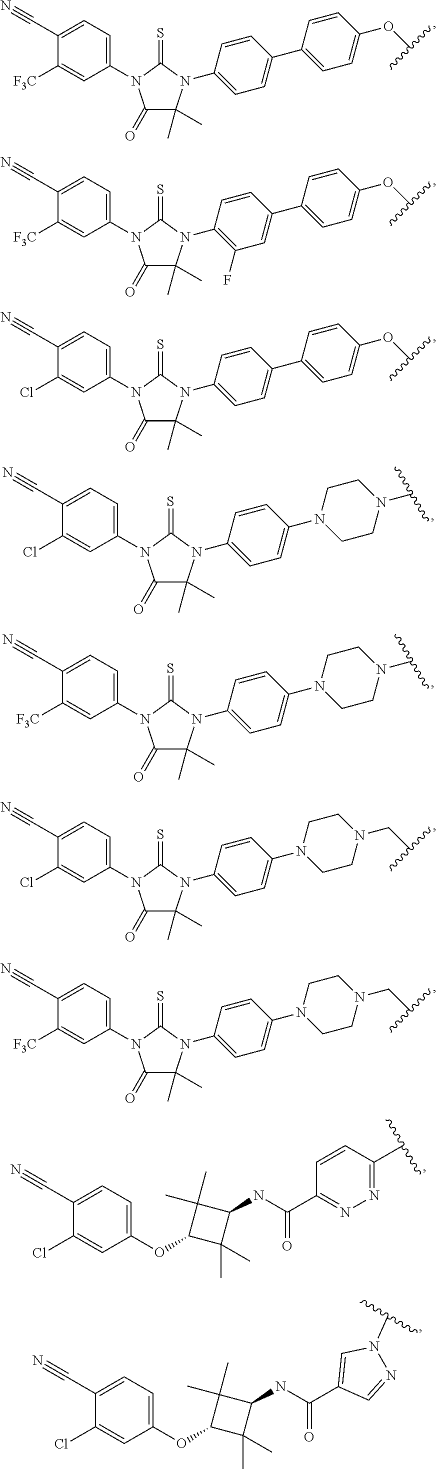

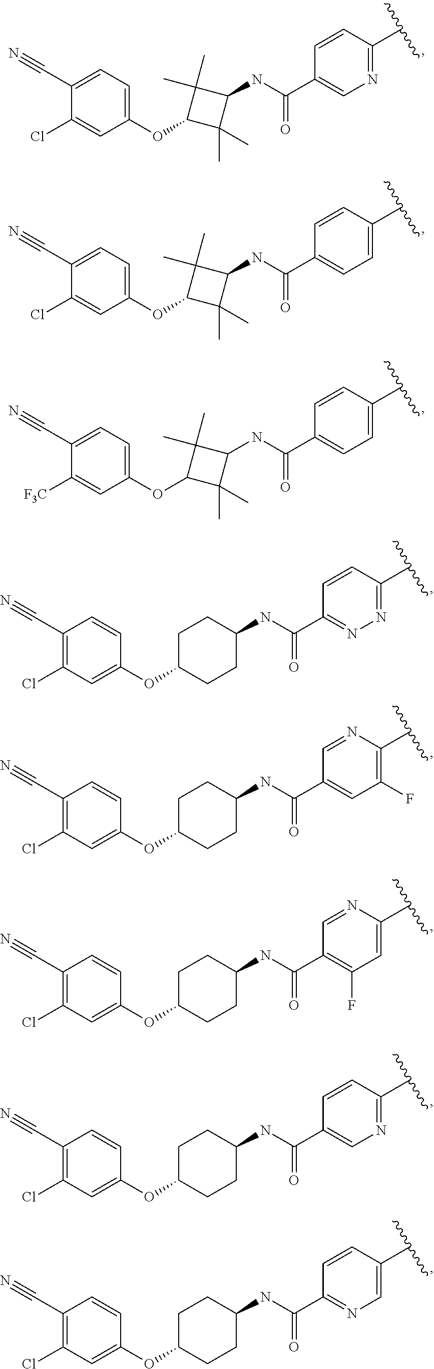

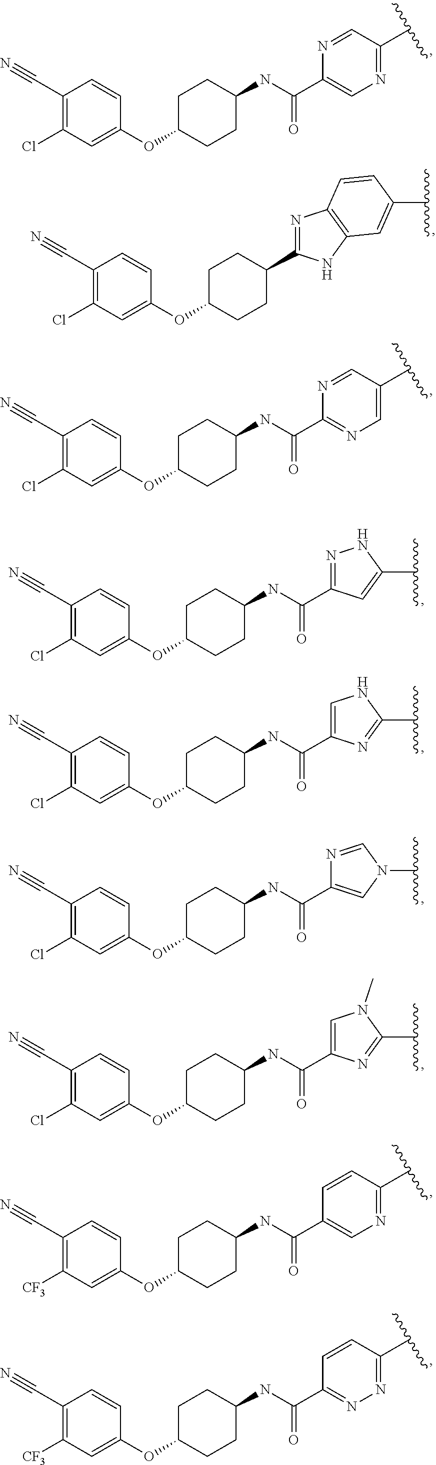

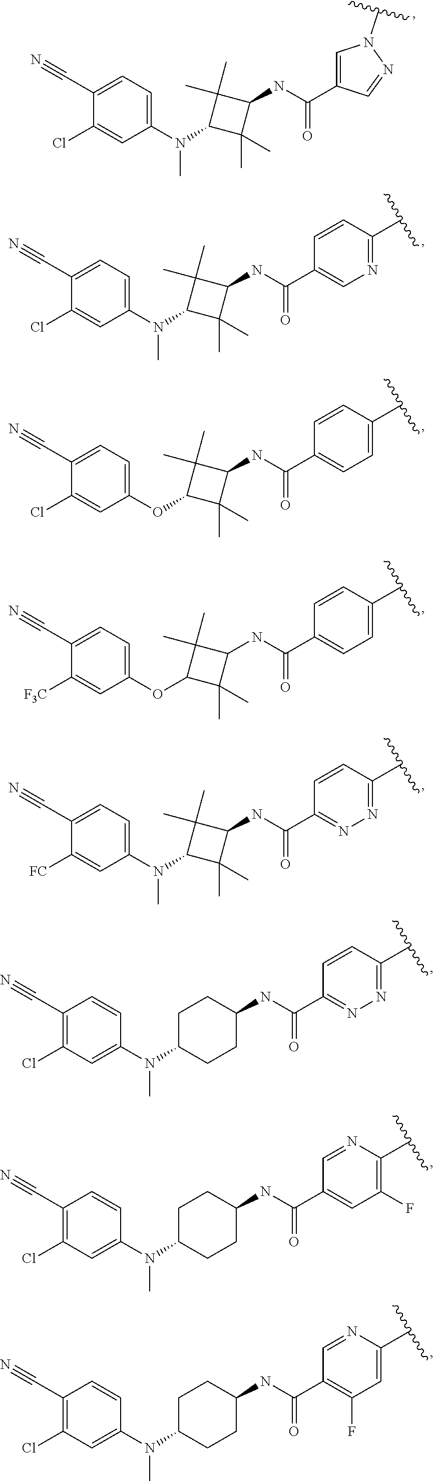

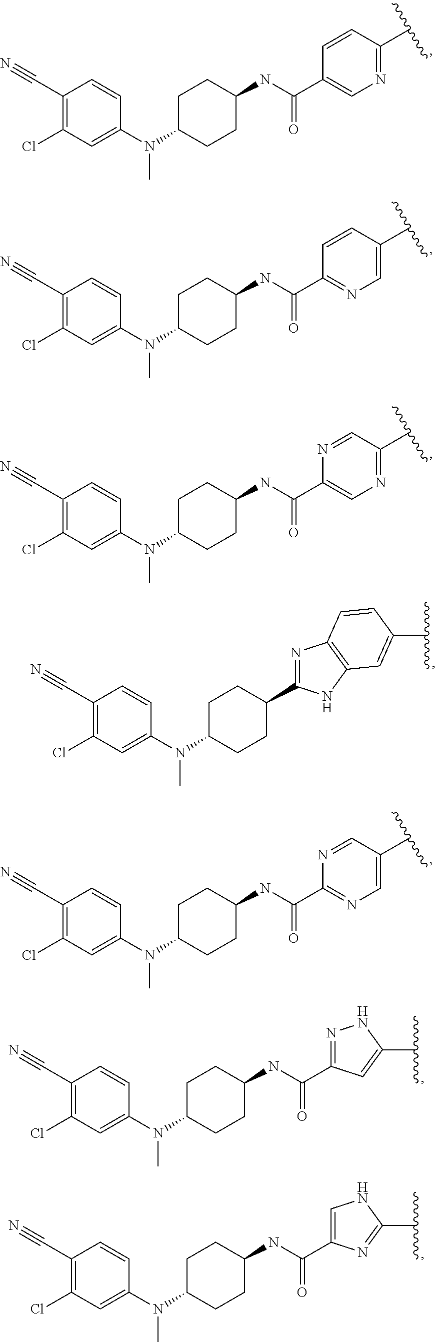

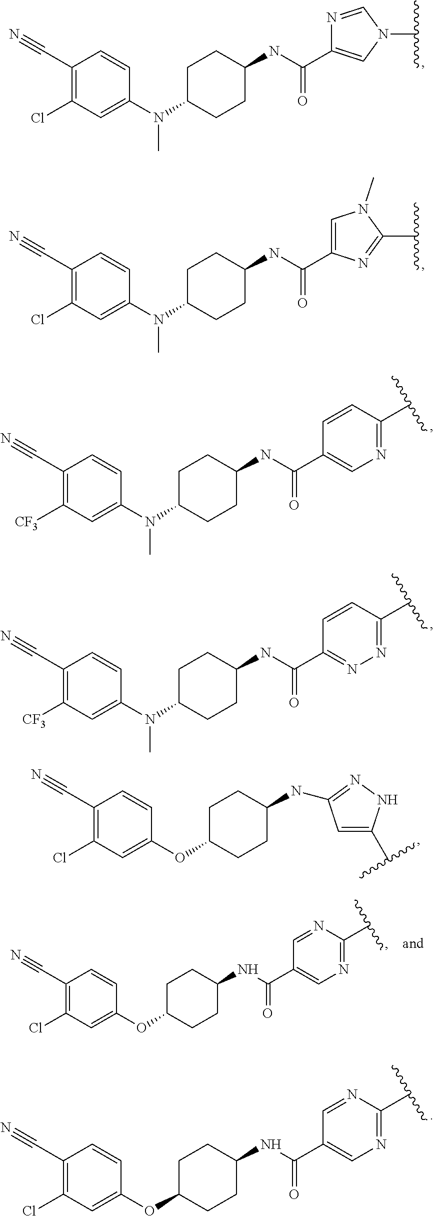

In some embodiments, the TBM is not selected from the group consisting of:

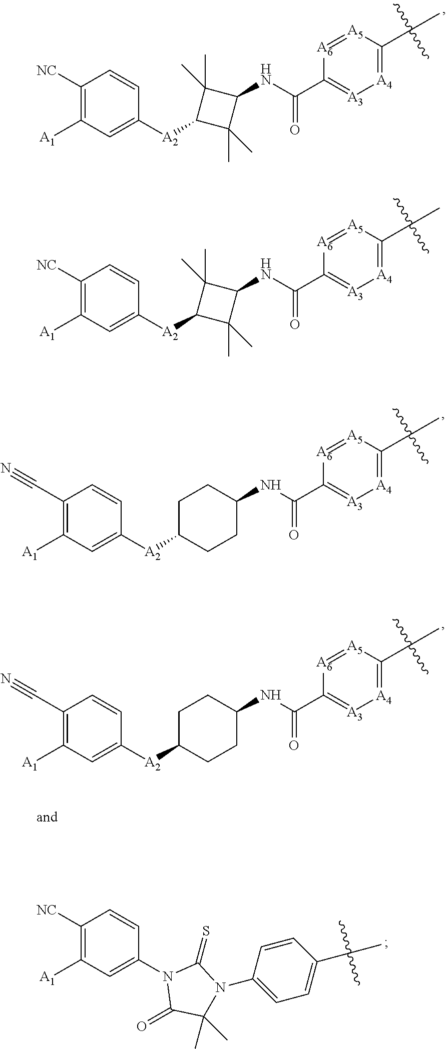

wherein A1 is selected from Cl, F, Br or CF3; A2 is selected from O, NH, N-methyl or N-ethyl; and A3, A4, A5 and A6 are each independently CH or N.

In some embodiments, the TBM is not selected from the group consisting of:

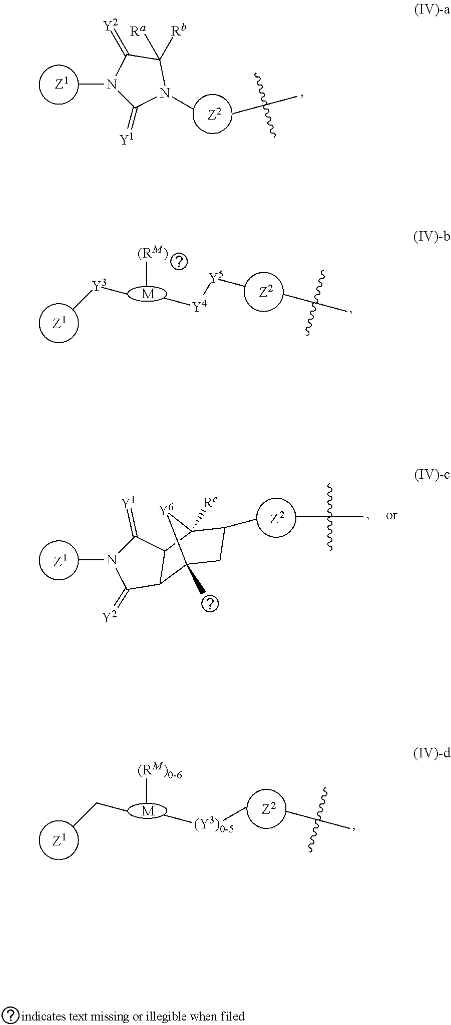

wherein Z1 is selected from the group consisting of an aryl group, a heteroaryl group, a bicyclic group, and a bi-heterocyclic group, each independently substituted by one or more substituents selected from the group consisting of a halo group, a hydroxyl group, a nitro group, CN, C≡CH, an unsubstituted C1-6 alkyl group, a C1-6 alkyl group substituted by a C1-6 alkoxyl group or one or more halo groups, an unsubstituted C1-6 alkoxyl group, a C1-6 alkoxyl group substituted by one or more halo groups, an unsubstituted C2-6 alkenyl, a C2-6 alkenyl substituted by one or more halo groups, an unsubstituted C2-6 alkynyl, and a C3-6 alkynyl substituted by one or more halo groups;
Y1, Y2, Y6 are each independently NRY1, O or S;
Y3, Y4, Y5 are each independently selected from the group consisting of a bond, O, NRY2, CRY1RY2, C═O, C═S, SO, SO2, a heteroaryl group, and an aryl group;
M is a 3- to 6-membered ring with 0 to 4 heteroatoms, which is unsubstituted or substituted by 1 to 6 RM groups;
each RM group is independently selected from the group consisting of H, an unsubstituted C1-6 alkyl group, a C1-6 alkyl group substituted by a C1-6 alkoxyl group or one or more halo groups, halogen, and a C1-6 alkoxy group; or two RM groups are taken together with the atom they are attached to and form a 3- to 8-membered ring system containing 0 to 2 heteroatoms;
Ra, Rb, Re, Rd, RY1, RY2 are each independently selected from the group consisting of H, an unsubstituted C1-6 alkyl group, a C1-6 alkyl group substituted by a C1-6 alkoxyl group or one or more halo groups, halogen, a C1-6 alkoxy group, a cyclic group, and a heterocyclic group; or Ra, Rb are taken together with the atom they are attached to and form a 3- to 8-membered ring system containing 0 to 2 heteroatoms;
Z2 is selected from the group consisting of a bond, a C1-6 alkyl group, a C1-6 heteroalkyl group, 0, an aryl group, a heteroaryl group, an alicyclic group, a heterocyclic group, a biheterocyclic group, a biaryl group, and a biheteroaryl group, each of which is unsubstituted or substituted by 1 to 10 RZ2 groups;
each R^{Z2} group is independently selected from the group consisting of H, halo, an unsubstituted C1-6 alkyl group, a C1-6 alkyl group substituted by one or more F, —ORZ2A, a C3-6 cycloalkyl group, a C4-6 cycloheteroalkyl group, an unsubstituted C1-6 alkyl group, a C1-6 alkyl group substituted by a C1-3 alkyl group or a C1-6 alkoxyl group or one or more halo groups, an unsubstituted heterocyclic group, a heterocyclic group substituted by a C1-3 alkyl group or a C1-6 alkoxyl group or one or more halo groups, an unsubstituted aryl group, an aryl group substituted by a C1-3 alkyl group or a C1-6 alkoxyl group or one or more halo groups, an unsubstituted heteroaryl group, a heteroaryl group substituted by a C1-3 alkyl group or a C1-6 alkoxyl group or one or more halo groups, a bicyclic hereoaryl group, an unsubstituted OC1-3 alkyl group, and a OC1-3 alkyl substituted by one or more groups selected from F, OH, NH₂, NRY1RY2 and CN; and
R^{Z2A} is selected from the group consisting of H, a C1-6 alkyl group, and a C1-6 heteroalkyl group, each of which is unsubstituted or substituted by a cycloalkyl group, a cycloheteroalkyl group, an aryl group, a heterocyclic group, a heteroaryl group, halo, or a OC1-3 alkyl group.

### Cereblon E3 Ubiquitin Ligase Binding Moiety

CRBN, a component of a cullin-RING ubiquitin ligase (CRL) complex, is the target of thalidomide (Girardini et al., 2019). After binding to CRBN, thalidomide and its analogs inhibit the activity of CRL4CRBN E3 ubiquitin ligase in human cells (Fink et al., 2018). As used herein, the term “cereblon E3 ubiquitin ligase binding moiety” or “CLM” refers to a compound, which associates with or binds to a ubiquitin ligase for recruitment of the corresponding ubiquitination machinery to the to-be-degraded target protein.

In some embodiments, the cereblon E3 ubiquitin ligase binding moiety (CLM) represented by Formula (II)-1:

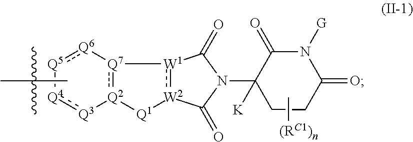

wherein:
W1 and W2 are each independently selected from C, CRC2 and N;
G is selected from the group consisting of H, OH, CH2OH, RC3OCOORC4, RC3OCONRC4RC5, and 2-(trimethylsilyl)ethoxymethyl group;
Q1 to Q7 are each independently C, O, S, N, CRC2 or NRC2; at least one of W1, W2, Q1, Q2, Q3, Q4, Q5, Q6 and Q7 comprises a heteroatom;
K is selected from the group consisting of H, an unsubstituted alkyl group, an alkyl group substituted by RC2, an unsubstituted cycloalkyl group, and a cycloalkyl group substituted by RC2; K is bound to the 6-membered ring with a stereospecific bond or a non-stereospecific bond;
RC1 is selected from the group consisting of an unsubstituted alkyl group, an alkyl group substituted by RC2, an unsubstituted aryl group, an aryl group substituted by RC2, an unsubstituted alkyl-aryl group, an alkyl-aryl group substituted by RC2, an unsubstituted alkoxy group, and an alkoxy group substituted by RC2;
RC2 is selected from the group consisting of H, halo, CH2OH, CRC4, NRC4RC5, 2-(trimethylsilyl)ethoxymethyl, an alkoxyl group, an unsubstituted alkyl group, an alkyl group substituted by one or more halo groups, an unsubstituted cycloalkyl group, a cycloalkyl group substituted by one or more halo groups, an unsubstituted aryl group, an aryl group substituted by one or more halo groups, an unsubstituted heteroaryl group, a heteroaryl group substituted by one or more halo groups, an unsubstituted heterocyclyl group, and a heterocyclyl group substituted by one or more halo groups;
RC3 is selected from the group consisting of an unsubstituted alkylene group, and an alkylene group substituted by RC2;
RC4 and RC5 are independently selected from the group consisting of an unsubstituted alkyl group, an alkyl group substituted by RC2, an unsubstituted cycloalkyl group, a cycloalkyl group substituted by RC2, an unsubstituted heterocyclyl group, a heterocyclyl group substituted by RC2, an unsubstituted aryl group, an aryl group substituted by RC2, an unsubstituted heteroaryl group, and a heteroaryl group substituted by RC2; and n is 0, 1, 2, 3 or 4, or a pharmaceutically acceptable salt or analog thereof.

In some embodiment, Q1 is NRC2. In some embodiment, RC2 is an unsubstituted C1-6 alkyl group, a C1-6 alkyl group substituted by one or more halo groups, an unsubstituted C1-6 cycloalkyl group, a C1-6 cycloalkyl group substituted by one or more halo groups. In some embodiment, RC2 is an unsubstituted C1-6 alkyl group. In some embodiment, RC2 is an unsubstituted C1-4 alkyl group. In some embodiment, RC2 is methyl or ethyl.

In some embodiment, W1 and W2 are each C and connected together through a double bond.

In some embodiment, Q2, Q3, Q4, Q5, Q6 and Q7 together form a benzene or heterobenzen ring, optionally substituted with one or more RC2. In some embodiment, Q2, Q3, Q4, Q5, Q6 and Q7 together form an unsubstituted benzene or heterobenzen ring. In some embodiment, Q2, Q3, Q4, Q5, Q6 and Q7 together form an unsubstituted benzene ring.

In some embodiment, K is H.

In some embodiment, n is 0, 1, or 2. In some embodiment, n is 0 or 1. In some embodiment, n is 0.

In some embodiment, G is selected from the group consisting of H, CH2OH, RC3OCOORC4, and RC3OCONRC4RC5. In some embodiment, G is selected from the group consisting of H, CH2OH, and RC3OCOORC4. In some embodiment, G is H. In some embodiment, G is CH2OH. In some embodiment, G is RC3OCOORC4. In some embodiment, RC3 is selected from the group consisting of an unsubstituted alkylene group. In some embodiment, RC3 is an unsubstituted C1-6 alkylene group. In some embodiment, RC3 is an unsubstituted C1-4 alkylene group. In some embodiment, RC3 is methylene or ethylene. In some embodiment, RC3 is methylene. In some embodiment, RC4 is an unsubstituted alkyl group or an alkyl group substituted by RC2. In some embodiment, RC4 is an unsubstituted alkyl group. In some embodiment, RC4 is an unsubstituted C1-6 alkyl group. In some embodiment, RC4 is an unsubstituted C1-4 alkyl group. In some embodiment, RC4 is methyl or ethyl. In some embodiment, RC4 is methyl.

In some embodiment, the CLM is represented with Formula (II)-2:

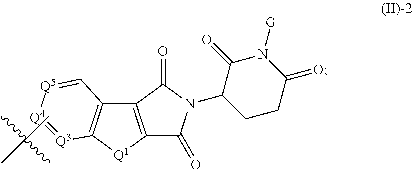

wherein
Q1 is O, S or NRC2;
Q3 to Q5 are each independently C or CRC2;
G is selected from the group consisting of H, OH, CH2OH, RC3OCOORC4 and 2-(trimethylsilyl)ethoxymethyl group;
RC2 is selected from the group consisting of H, CH2OH, CRC4, 2-(trimethylsilyl)ethoxymethyl, an unsubstituted C1-6 alkyl group, a C1-6 alkyl group substituted by one or more halo groups, a unsubstituted C1-6 cycloalkyl group, a C1-6 cycloalkyl group substituted by one or more halo groups;
RC3 is selected from the group consisting of a methylene group and a methylene group substituted by RC2;
RC4 is selected from the group consisting of an unsubstituted alkyl group, an alkyl group substituted by RC2, an unsubstituted cycloalkyl group, a cycloalkyl group substituted by RC2, an unsubstituted heterocyclyl group, a heterocyclyl group substituted by RC2.

In some embodiment, Q1 is NRC2. In some embodiment, RC2 is an unsubstituted C1-6 alkyl group, a C1-6 alkyl group substituted by one or more halo groups, an unsubstituted C1-6 cycloalkyl group, a C1-6 cycloalkyl group substituted by one or more halo groups. In some embodiment, RC2 is an unsubstituted C1-6 alkyl group. In some embodiment, RC2 is an unsubstituted C1-4 alkyl group. In some embodiment, RC2 is methyl or ethyl.

In some embodiment, Q3-Q5 are each C.

In some embodiment, G is selected from the group consisting of H, CH2OH, RC3OCOORC4, and RC3OCONRC4RC5. In some embodiment, G is selected from the group consisting of H, CH2OH, and RC3OCOORC4. In some embodiment, G is H. In some embodiment, G is CH2OH. In some embodiment, G is RC3OCOORC4. In some embodiment, RC3 is selected from the group consisting of an unsubstituted alkylene group. In some embodiment, RC3 is an unsubstituted C1-6 alkylene group. In some embodiment, RC3 is an unsubstituted C1-4 alkylene group. In some embodiment, RC3 is methylene or ethylene. In some embodiment, RC3 is methylene. In some embodiment, RC4 is an unsubstituted alkyl group or an alkyl group substituted by R_C2. In some embodiment, RC4 is an unsubstituted alkyl group. In some embodiment, RC4 is an unsubstituted C1-6 alkyl group. In some embodiment, RC4 is an unsubstituted C1-4 alkyl group. In some embodiment, RC4 is methyl or ethyl. In some embodiment, RC4 is methyl.

Without wishing to be bound by any particular theory, the present disclosure recognizes that, in some embodiments, the bivalent compound disclosed herein with the CLM including two carbonyls adjacent to the nitrogen connected to the 2,6-dioxo-piperidine ring provides improved E3 ligase binding, protein ubiquitination, and/or target protein degradation over a control compound with the CLM including only one carbonyl adjacent to either side of the nitrogen connected to the 2,6-dioxo-piperidine ring.

### Optional Linker Moiety

As used herein, a “linker” or “linker moiety” is a molecular structure capable of connecting two separate moieties to one another through covalent bonds. In some embodiments, linkers provide for desirable spacing of the two entities. The term “linker” in some aspects refers to any agent or molecule that bridges the TBM to the CLM. The present disclosure recognizes suitable sites for attaching a linker, provided that the linker, once attached to the conjugate of the present disclosures, does not interfere with the function of the TBM, i.e., its ability to bind POI, or the function of the CLM, i.e., its ability to recruit a ubiquitin ligase.

The length of the linker of the bivalent compound can be adjusted to minimize the molecular weight of the bivalent compounds, avoid the clash of the TBM or targeting moiety with the ubiquitin ligase. In certain embodiments, the linker comprises acyclic or cyclic saturated or unsaturated carbon, ethylene glycol, amide, amino, ether, urea, carbamate, aromatic, heteroaromatic, heterocyclic or carbonyl groups. In certain embodiments, the length of the linker is 0, 1, 2, 3, 4, 5, 6, 7, 8, 9, 10, 11, 12, 13, 14, 15, 16, 17, 18, 19, 20 or more atoms.

In some embodiments, In some embodiment, the TBM is connected to the CLM through a bond or a linker moiety (L). In some embodiment, the TBM is connected to the CLM through a linker moiety (L). In some embodiment, the TBM is connected to the CLM through Q5. In some embodiment, the TBM is connected to the CLM through Q4. In some embodiment, the TBM is connected to the CLM through Q3.

In some embodiment, the linker moiety is of Formula (III):

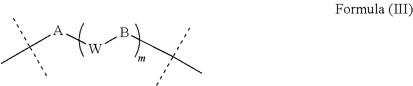

wherein
A, W, and B, at each occurrence, are independently selected from null, CO, CO₂, C(O)NR¹, C(S)NR¹, O, S, SO, SO₂, SO₂NR¹, NR¹, NR¹CO, NR¹CONR², NR¹C(S), optionally substituted C₁-C₈ alkyl, optionally substituted C₁-C₈ alkoxy, optionally substituted C₁-C₈alkoxyC₁-C₈alkyl, optionally substituted C₁-C₈ haloalkyl, optionally substituted C₁-C₈ hydroxyalkyl, optionally substituted C₂-C₈ alkenyl, optionally substituted C₂-C₈ alkynyl, optionally substituted C₃-C₈ cycloalkyl, optionally substituted C₃-C₈ cycloalkoxy, optionally substituted 3-8 membered heterocyclyl, optionally substituted aryl, optionally substituted heteroaryl, optionally substituted C₃-C₁₃ fused cycloalkyl, optionally substituted C₃-C₁₃ fused heterocyclyl, optionally substituted C₃-C₁₃ bridged cycloalkyl, optionally substituted C₃-C₁₃ bridged heterocyclyl, optionally substituted C₃-C₁₃ spiro cycloalkyl, and optionally substituted C₃-C₁₃ spiro heterocyclyl, wherein
   R¹ and R² are independently selected from hydrogen, optionally substituted C₁-C₆ alkyl, optionally substituted C₃-C₆ cycloalkyl, optionally substituted C₃-C₆ cycloalkoxy, optionally substituted 3-6 membered heterocyclyl, optionally substituted C₁-C₆ alkoxy, optionally substituted C₁-C₆ alkoxyalkyl, optionally substituted C₁-C₆ haloalkyl, optionally substituted C₁-C₆ hydroxyalkyl, optionally substituted C₁-C₆ alkylamino, and optionally substituted C₁-C₆alkylaminoC₁-C₆alkyl; and
m is 0 to 15.

In some embodiment, the linker moiety is of Formula (III)-1:

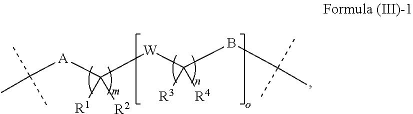

wherein
R¹, R², R³, and R⁴, at each occurrence, are independently selected from hydrogen, halogen, CN, OH, NH₂, optionally substituted C₁-C₆ alkyl, optionally substituted C₃-C₆ cycloalkyl, optionally substituted C₃-C₆ cycloalkoxy, optionally substituted 3-6 membered heterocyclyl, optionally substituted C₁-C₆ alkoxy, optionally substituted C₁-C₆ alkoxyalkyl, optionally substituted C₁-C₆ haloalkyl, optionally substituted C₁-C₆ hydroxyalkyl, optionally substituted C₁-C₆ alkylamino, and optionally substituted C1-C₆alkylaminoC₁-C₆alkyl;
A, W, and B, at each occurrence, are independently selected from null, CO, CO₂, C(O)NR⁵, C(S)NR⁵, O, S, SO, SO₂, SO₂NR⁵, NR⁵, NR⁵CO, NR⁵CONR⁶, NR⁵C(S), optionally substituted C₁-C₈ alkyl, optionally substituted C₁-C₈ alkoxy, optionally substituted C₁-C₈alkoxyC₁-C₈alkyl, optionally substituted C₁-C₈ haloalkyl, optionally substituted C₁-C₈ hydroxyalkyl, optionally substituted C₂-C₈ alkenyl, optionally substituted C₂-C₈ alkynyl, optionally substituted C₃-C₈ cycloalkyl, optionally substituted C₃-C₈ cycloalkoxy, optionally substituted 3-8 membered heterocyclyl, optionally substituted aryl, optionally substituted heteroaryl, optionally substituted C₃-C₁₃ fused cycloalkyl, optionally substituted C₃-C₁₃ fused heterocyclyl, optionally substituted C₃-C₁₃ bridged cycloalkyl, optionally substituted C₃-C₁₃ bridged heterocyclyl, optionally substituted C₃-C₁₃ spiro cycloalkyl, and optionally substituted C₃-C₁₃ spiro heterocyclyl, wherein
   R⁵ and R⁶ are independently selected from hydrogen, optionally substituted C₁-C₆ alkyl, optionally substituted C₃-C₆ cycloalkyl, optionally substituted C₃-C₆ cycloalkoxy, optionally substituted 3-6 membered heterocyclyl, optionally substituted C₁-C₆ alkoxy, optionally substituted C₁-C₆ alkoxyalkyl, optionally substituted C₁-C₆ haloalkyl, optionally substituted C₁-C₆ hydroxyalkyl, optionally substituted C₁-C₆ alkylamino, and optionally substituted C₁-C₆alkylaminoC₁-C₆alkyl;
m is 0 to 15;
each n is 0 to 15; and
is 0 to 15.

In some embodiment, the linker moiety is of Formula (III)-2:

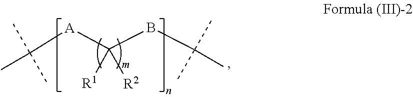

wherein
each R¹, and each R² are independently selected from hydrogen, halogen, CN, OH, NH₂, and optionally substituted C₁-C₆ alkyl, optionally substituted C₃-C₆ cycloalkyl, optionally substituted C₃-C₆ cycloalkoxy, optionally substituted 3-6 membered heterocyclyl, optionally substituted C₁-C₆ alkoxy, optionally substituted C₁-C₆ alkoxyalkyl, optionally substituted C₁-C₆ haloalkyl, optionally substituted C₁-C₆ hydroxyalkyl, optionally substituted C₁-C₆ alkylamino, or C₁-C₆alkylaminoC₁-C₆alkyl;
each A and each B are independently selected from null, CO, CO₂, C(O)NR³, C(S)NR³, O, S, SO, SO₂, SO₂NR³, NR³, NR³CO, NR³CONR⁴, NR³C(S), and optionally substituted C₁-C₈ alkyl, optionally substituted C₁-C₈ alkoxy, optionally substituted C₁-C₈alkoxyC₁-C₈alkyl, optionally substituted C₁-C₈ haloalkyl, optionally substituted C₁-C₈ hydroxyalkyl, optionally substituted C₂-C₈ alkenyl, optionally substituted C₂-C₈ alkynyl, optionally substituted C₃-C₈ cycloalkyl, optionally substituted C₃-C₈ cycloalkoxy, optionally substituted 3-8 membered heterocyclyl, optionally substituted aryl, optionally substituted heteroaryl, optionally substituted C₃-C₁₃ fused cycloalkyl, optionally substituted C₃-C₁₃ fused heterocyclyl, optionally substituted C₃-C₁₃ bridged cycloalkyl, optionally substituted C₃-C₁₃ bridged heterocyclyl, optionally substituted C₃-C₁₃ spiro cycloalkyl, or C₃-C₁₃ spiro heterocyclyl, wherein
R³ and R⁴ are independently selected from hydrogen, and optionally substituted C₁-C₆ alkyl, optionally substituted C₃-C₆ cycloalkyl, optionally substituted C₃-C₆ cycloalkoxy, optionally substituted 3-6 membered heterocyclyl, optionally substituted C₁-C₆ alkoxy, optionally substituted C₁-C₆ alkoxyalkyl, optionally substituted C₁-C₆ haloalkyl, optionally substituted C₁-C₆ hydroxyalkyl, optionally substituted C₁-C₆ alkylamino, or C₁-C₆alkylaminoC₁-C₆alkyl;
each m is 0 to 15; and
n is 0 to 15.

In some embodiment, the linker moiety is of FORMULA (III)-3:

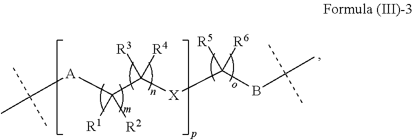

wherein
X is selected from O, NH, and NR⁷;
R¹, R², R³, R⁴, R⁵, and R⁶, at each occurrence, are independently selected from hydrogen, halogen, CN, OH, NH₂, optionally substituted C₁-C₆alkyl, optionally substituted C₃-C₆ cycloalkyl, optionally substituted C₃-C₆ cycloalkoxy, optionally substituted 3-6 membered heterocyclyl, optionally substituted C₁-C₆ alkoxy, optionally substituted C₁-C₆ alkoxyalkyl, optionally substituted C₁-C₆ haloalkyl, optionally substituted C₁-C₆ hydroxyalkyl, optionally substituted C₁-C₆ alkylamino, and optionally substituted C1-C₆alkylaminoC₁-C₆alkyl; A and B, at each occurrence, are independently selected from null, CO, CO₂, C(O)NR⁷, C(S)NR⁷, 0, S, SO, SO₂, SO₂NR⁷, NR⁷, NR⁷CO, NR⁷CONR⁸, NR⁷C(S), optionally substituted C₁-C₈ alkyl, optionally substituted C₁-C₈ alkoxy, optionally substituted C₁-C₈alkoxyC₁-C₈alkyl, optionally substituted C₁-C₈ haloalkyl, optionally substituted C₁-C₈ hydroxyalkyl, optionally substituted C₂-C₈ alkenyl, optionally substituted C₂-C₈ alkynyl, optionally substituted C₃-C₈ cycloalkyl, optionally substituted C₃-C₈ cycloalkoxy, optionally substituted 3-8 membered heterocyclyl, optionally substituted aryl, optionally substituted heteroaryl, optionally substituted C₃-C₁₃ fused cycloalkyl, optionally substituted C₃-C₁₃ fused heterocyclyl, optionally substituted C₃-C₁₃ bridged cycloalkyl, optionally substituted C₃-C₁₃ bridged heterocyclyl, optionally substituted C₃-C₁₃ spiro cycloalkyl, and optionally substituted C₃-C₁₃ spiro heterocyclyl, wherein
R⁸ and R⁸ are independently selected from hydrogen, optionally substituted C₁-C₆alkyl, optionally substituted C₃-C₆ cycloalkyl, optionally substituted C₃-C₆ cycloalkoxy, optionally substituted 3-6 membered heterocyclyl, optionally substituted C₁-C₆ alkoxy, optionally substituted C₁-C₆ alkoxyalkyl, optionally substituted C₁-C₆ haloalkyl, optionally substituted C₁-C₆ hydroxyalkyl, optionally substituted C₁-C₆ alkylamino, and optionally substituted C₁-C₆alkylaminoC₁-C₆alkyl;
each m is 0 to 15;
each n is 0 to 15;
o is 0 to 15; and
p is 0 to 15.

In some embodiment, the linker moiety comprises a 3 to 13 membered ring, a 4 to 13 membered fused ring, a 5 to 13 membered bridged ring, and a 5 to 13 membered spiro ring. In some embodiment, the linker moiety comprises a ring selected from the group consisting of Formula C1, C2, C3, C4 and C5:

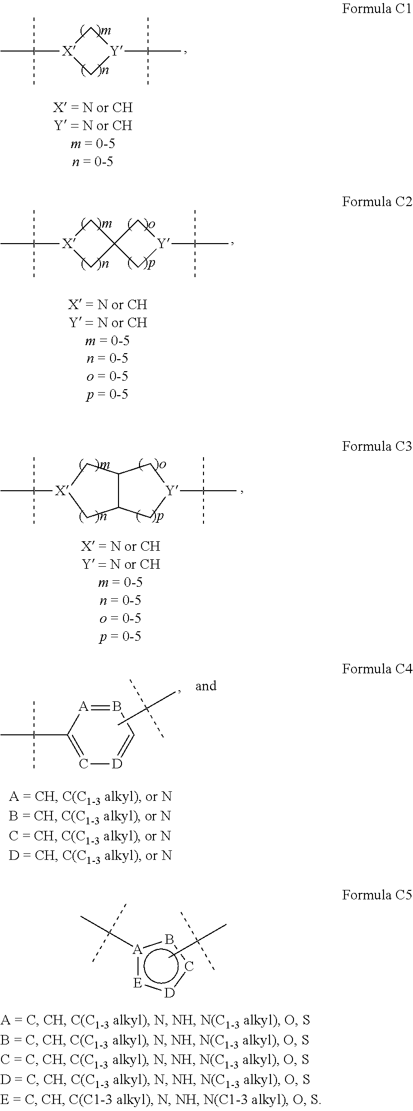

In some embodiment, the linker moiety is of Formula (IV):

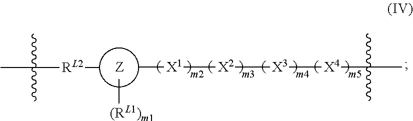

wherein
Z is selected from the group consisting of a 3- to 8-membered ring, a 5- to 12-membered bicyclic ring, an 8- to 15-membered tricyclic ring and a 6- to 12-membered spiro bicyclic ring, each independently having 0-4 heteroatoms;
RL1 is selected from the group consisting of H, an unsubstituted C₁₋₆ alkyl group, a C₁₋₆ alkyl group substituted by a C₁₋₆ alkoxyl group or one or more halo groups, halogen, a C₁₋₆ alkoxy group, a keto group, or an oxide group; wherein, two RL1 groups are optionally taken together to form a 3-8 membered ring system containing 0-2 heteroatoms; RL2 is a bond or an ethynylene group;
X1 is selected from the group consisting of a methylene group and an ethylene group;
X2 and X4 are independently selected from the group consisting of a methylene group, C(═O), C(═O)C(═O), CONRL3, NRL3C(═O), NRL3C(═O)NRL4, NRL3C(═O)C(═O)NRL4, NRL3SO2NRL4, SO2NRL3, CRL3RL4, NRL5, O and S;
X3 is selected from the group consisting of an unsubstituted C₁₋₈ alkylene group, a C₁₋₈ alkylene group substituted by 1 to 6 RL1, an unsubstituted C1-8 heteroalkylene group, a C1-8 heteroalkylene group substituted by 1 to 6 RL1, an unsubstituted 3- to 8-membered arylene group, a 3- to 8-membered arylene group substituted by 1 to 6 RL1, an unsubstituted 3- to 8-membered heteroarylene group with 1 to 3 hetero atoms, and a 3- to 8-membered heteroarylene group with 1 to 3 hetero atoms and substituted by 1 to 6 RL1, an unsubstituted 3- to 7-membered cyclic alkylene group, a 3- to 7-membered cyclic alkylene group substituted by 1 to 6 RL1, an unsubstituted 3- to 7-membered heterocyclic alkylene group with 1 to 2 hetero atoms, and a 3- to 7-membered heterocyclic alkylene group with 1 to 2 hetero atoms and substituted by 1 to 6 RL1; or, X3 is a 6- to 12-membered spiro bicyclic ring having 0-4 heteroatoms;
RL3 is hydrogen;
RL4 and RL5 are independently selected from the group consisting of H, an unsubstituted C1-6 alkyl group, and a C1-6 alkyl group substituted by a C1-6 alkoxyl group or one or more halo groups;
m1 is 0, 1, 2, 3, 4, 5 or 6;
m2 is 0 or 1;
m3 is 0 or 1;
m4 is 1; and
m5 is 0 or 1.

In some embodiment, the heteroatoms in Formula (IV) are each independently selected N, O and S.

In some embodiment, the Z ring comprises one or two heteroatoms selected from N, O and S. In some embodiment, Z is selected from the group consisting of a 3- to 8-membered ring, a 6- to 10-membered bicyclic ring and a 8- to 10-membered spiro bicyclic ring, each independently having 1-2 heteroatoms.

In some embodiment, RL1 is selected from the group consisting of H, an unsubstituted linear C₁₋₃ alkyl group, a keto group, or an oxide group;

In some embodiment, RL2 is a bond or an ethynylene group.

In some embodiment, X1 is selected from the group consisting of a methylene group and an ethylene.

In some embodiment, X2 is selected from the group consisting of C(═O), NRL3C(═O) and O.

In some embodiment, X3 is selected from the group consisting of an unsubstituted C1-6 alkylene group, an unsubstituted 3- to 7-membered cyclic alkylene group, and an unsubstituted 3- to 7-heterocyclic alkylene group with 0 to 2 hetero atoms; or, X3 is a 10- to 12-membered spiro bicyclic ring having 0-2 heteroatoms.

In some embodiment, X4 is selected from the group consisting of a methylene group, CONRL3 and O.

In some embodiment, m1 is 0, 1, 2 or 3.

In some embodiment, RL1 is selected from the group consisting of H, an unsubstituted C1-3 alkyl group, a C1-3 alkyl group substituted by a C1-3 alkoxyl group or one or more halo groups, halogen, a C1-3 alkoxy group, a keto group, or an oxide group; wherein, two RL1 groups are optionally taken together to form a 3-8 membered ring system containing 0-2 heteroatoms. In some embodiment, RL1 is an oxide group which is attached to one heteroatom N on the Z ring to form an N-oxide group (N⁺—O⁻). In some embodiment, RL1 is an alkyl selected from the group consisting of a linear C1-6 alkyl and a branched C1-6 alkyl, each of which is unsubstituted or substituted by a C1-6 alkoxyl group or one or more halo groups. In some embodiment, RL1 is an alkyl selected from the group consisting of a linear C1-3 alkyl and a branched C1-3 alkyl, each of which is unsubstituted or substituted by a C1-6 alkoxyl group or one or more halo groups.

In some embodiment, X3 is selected from the group consisting of a bond, an unsubstituted C1-6 alkylene group, a C1-6 alkylene group substituted by 1 to 6 RL1, an unsubstituted C1-6 heteroalkylene group, a C1-6 heteroalkylene group substituted by 1 to 6 RL1. In some embodiment, X3 is selected from the group consisting of an unsubstituted C1-5 alkylene group.

In some embodiment, RL4 and RL5 are independently an alkyl selected from the group consisting of a linear C1-6 alkyl and a branched C1-6 alkyl, each of which is unsubstituted or substituted by a C1-6 alkoxyl group or one or more halo groups. In some embodiment, RL4 and RL5 are independently an alkyl selected from the group consisting of a linear C1-3 alkyl and a branched C1-3 alkyl, each of which is unsubstituted or substituted by a C1-6 alkoxyl group or one or more halo groups.

In some embodiment, the linker moiety is selected from the group consisting of:

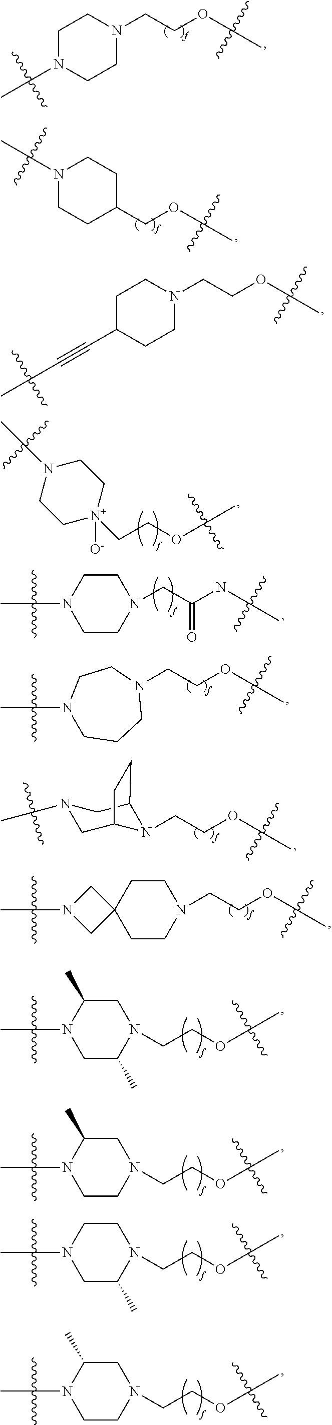

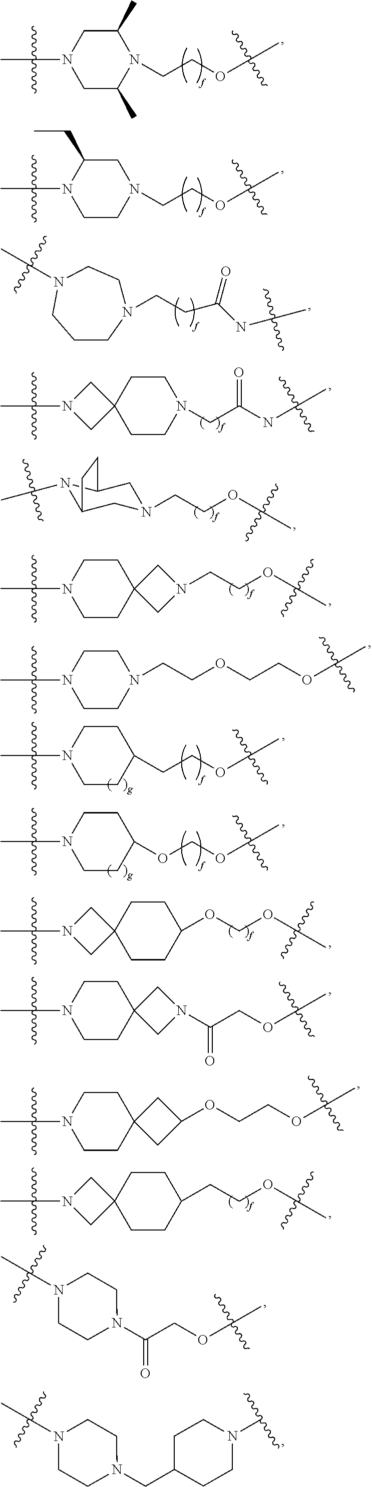

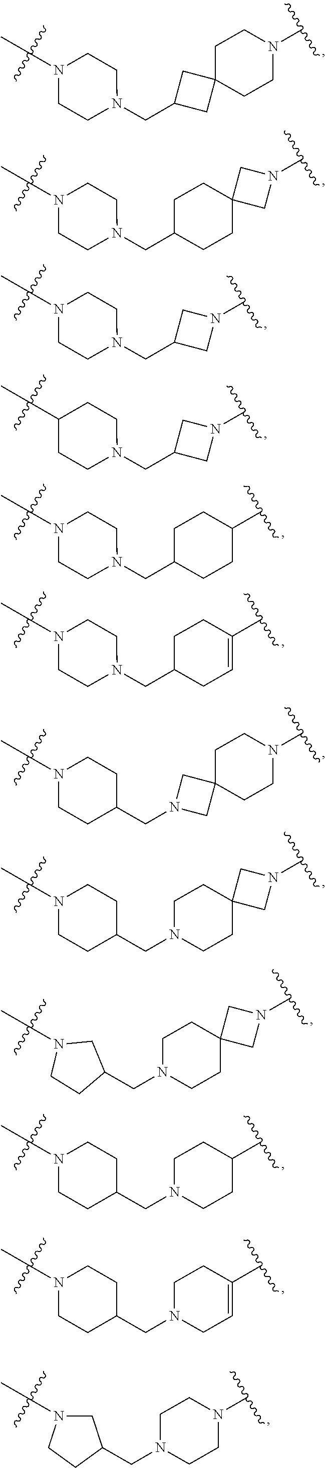

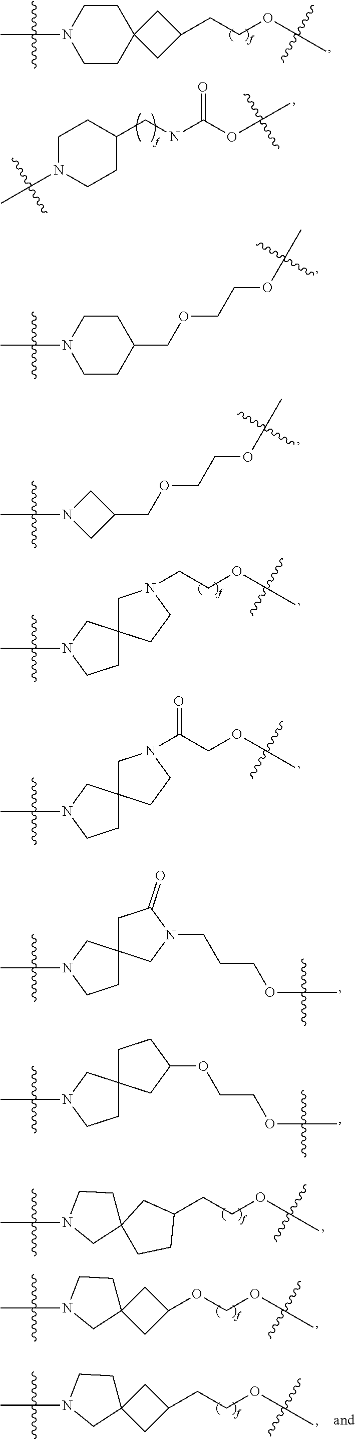

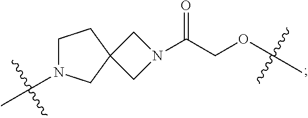

wherein f is an integer of 0, 1, 2, 3 or 4; and g is an integer of 0, 1, 2 or 3.

### Synthesis and Testing of Bivalent Compounds

The binding affinity of novel synthesized bivalent compounds can be assessed using standard biophysical assays known in the art (e.g., isothermal titration calorimetry (ITC), surface plasmon resonance (SPR)). Cellular assays can then be used to assess the bivalent compound's ability to induce target protein degradation. Besides evaluating a bivalent compound's induced changes in the protein levels POI, POI mutants, or POI fusion proteins, enzymatic activity can also be assessed. Assays suitable for use in any or all of these steps are known in the art, and include, e.g., western blotting, quantitative mass spectrometry (MS) analysis, flow cytometry, enzymatic activity assay, ITC, SPR, cell growth inhibition, xenograft, orthotopic, and patient-derived xenograft models. Suitable mouse models for use in any or all of these steps are known in the art and include subcutaneous xenograft models, orthotopic models, patient-derived xenograft models, and patient-derived orthotopic models.

By way of non-limiting example, detailed synthesis protocols are described in the Examples for specific exemplary bivalent compounds.

Pharmaceutically acceptable isotopic variations of the compounds disclosed herein are contemplated and can be synthesized using conventional methods known in the art or methods corresponding to those described in the Examples (substituting appropriate reagents with appropriate isotopic variations of those reagents). Specifically, an isotopic variation is a compound in which at least one atom is replaced by an atom having the same atomic number, but an atomic mass different from the atomic mass usually found in nature. Useful isotopes are known in the art and include, for example, isotopes of hydrogen, carbon, nitrogen, oxygen, phosphorus, sulfur, fluorine, and chlorine. Exemplary isotopes thus include, e.g., ²H, ³H, ¹³C, ¹⁴C, ¹⁵N, ¹⁷O, ¹⁸O, ³²P, ³⁵S, ¹⁸F, and ³⁶Cl.

Isotopic variations (e.g., isotopic variations containing ²H) can provide therapeutic advantages resulting from greater metabolic stability, e.g., increased in vivo half-life or reduced dosage requirements. In addition, certain isotopic variations (particularly those containing a radioactive isotope) can be used in drug or substrate tissue distribution studies. The radioactive isotopes tritium (³H) and carbon-14 (¹⁴C) are particularly useful for this purpose in view of their ease of incorporation and ready means of detection.

Pharmaceutically acceptable solvates of the compounds disclosed herein are contemplated. A solvate can be generated, e.g., by substituting a solvent used to crystallize a compound disclosed herein with an isotopic variation (e.g., D₂O in place of H₂O, d₆-acetone in place of acetone, or d₆-DMSO in place of DMSO).

Pharmaceutically acceptable fluorinated variations of the compounds disclosed herein are contemplated and can be synthesized using conventional methods known in the art or methods corresponding to those described in the Examples (substituting appropriate reagents with appropriate fluorinated variations of those reagents). Specifically, a fluorinated variation is a compound in which at least one hydrogen atom is replaced by a fluoro atom. Fluorinated variations can provide therapeutic advantages resulting from greater metabolic stability, e.g., increased in vivo half-life or reduced dosage requirements.

Pharmaceutically acceptable prodrugs of the compounds disclosed herein are contemplated and can be synthesized using conventional methods known in the art or methods corresponding to those described in the Examples (e.g., converting hydroxyl groups or carboxylic acid groups to ester groups). As used herein, a “prodrug” refers to a compound that can be converted via some chemical or physiological process (e.g., enzymatic processes and metabolic hydrolysis) to a therapeutic agent. Thus, the term “prodrug” also refers to a precursor of a biologically active compound that is pharmaceutically acceptable. A prodrug may be inactive when administered to a subject, i.e. an ester, but is converted in vivo to an active compound, for example, by hydrolysis to the free carboxylic acid or free hydroxyl. The prodrug compound often offers advantages of solubility, tissue compatibility or delayed release in an organism. The term “prodrug” is also meant to include any covalently bonded carriers, which release the active compound in vivo when such prodrug is administered to a subject. Prodrugs of an active compound may be prepared by modifying functional groups present in the active compound in such a way that the modifications are cleaved, either in routine manipulation or in vivo, to the parent active compound. Prodrugs include compounds wherein a hydroxy, amino or mercapto group is bonded to any group that, when the prodrug of the active compound is administered to a subject, cleaves to form a free hydroxy, free amino or free mercapto group, respectively. Examples of prodrugs include, but are not limited to, acetate, formate and benzoate derivatives of an alcohol or acetamide, formamide and benzamide derivatives of an amine functional group in the active compound and the like.

### Characterization of Bivalent Compounds

Compared with traditional protein inhibitors that mainly rely on binary interactions between small-molecule ligands and target proteins, PROTAC degraders work by involving more complicated three-body binding equilibria. Besides determining binary protein-ligand interaction, characterizing protein-protein interaction (PPI), ternary complex formation, ternary complex stability and cooperativity are also important in elucidating the SAR. The SAR obtained from cell-free biochemical assays can provide valuable feedback to rational lead optimization. Increasing evidence has also shown that ternary complex formation, ternary complex stability and cooperativity of PPI are more predictive of a PROTAC's degradation activity than its binary interactions. A ternary complex can be characterized by several different assays. Common biochemical and biophysical assays used to profile ternary complex formation, population, stability, binding affinities, cooperativity or kinetics are discussed below:

Fluorescence Polarization Assay

The fluorescence polarization (FP) assay can, in principle, be used to quantitatively analyze the binding of any small soluble fluorescent molecule (or any molecules that compete with it) to a protein. It is based on the principle that the degree of FP of a fluorescent molecule is proportional to its rotational relaxation time (ρ) which can be described by the Stokes equation:

ρ=3ηVRT

Where ρ is the rotation relaxation time, defined as the time taken by the fluorescent molecule to rotate 68.5°; V is molecular volume including hydration; η represents viscosity; R and T are the gas constant and thermodynamic temperatures, respectively.

Interaction of a large molecule (protein) with a fluorescent ligand (tracer) can change the effective molecular volume of the fluorescent ligand, and thus alter its rotational relaxation time and eventually its polarization that can be detected through plane-polarized light (FIG. 5). As such, the binding affinity between a fluorescent ligand and a protein can be monitored using plane-polarized light in a nondestructive and separation-free manner.

The FP assay is an economical and homogeneous assay that can provide rapid screening for a large number of compounds. As such, the FP assay has been extensively used in high-throughput screening (HTS) programs in drug discovery. In the PROTAC field, the FP assay provides a powerful tool to determine binary binding affinities, ternary binding affinities and cooperativities. The FP assay is an accessible assays that can be easily set up with various protocols available. The fluorescent probe is the key for the FP assay, which can be synthesized by a wide range of commercial services and academic core facilities or can be prepared in-house by attaching a fluorophore to a ligand. Various fluorophores are available, with fluorescein and rhodamine being used most commonly. Fluorescent probes are preferred to be as small as possible to maximize the molecular weight difference between a fluorescent probe and its binding protein. The binding affinity of a fluorescent probe to its target protein is also important, because the higher the binding affinity of a fluorescent probe, the wider the range of inhibitor potencies that can be resolved. However, the affinity of a fluorescent probe will be considered too high if the instrument cannot detect the fluorescent signal when it is lower than twice the Kd. The application of the FP assay for characterizing the binary binding affinity of PROTACs to their target protein or E3 ligase is the same as that of a small-molecule ligand. However, when repurposed for ternary binding affinity or cooperativity testing, the FP assay requires saturating the PROTAC with one binding protein first, then titrating into the other protein as a binary complex. As such, a large amount of the proteins are required, making it infeasible for HTS.

Amplified Luminescent Proximity Homogeneous Assay

Amplified luminescent proximity homogeneous assay (ALPHA) technology is a bead-based proximity assay that can be used to study interactions between molecules in a microplate format. In this assay, one binding partner (A) is attached to the donor bead, the other binding partner (B) is conjugated to the acceptor bead. The donor beads are coated with photosensitizer that can convert ambient O2 to singlet O2 (1O2) once illuminated at 680 nm. The singlet O2 has a half-life of 4 s, allowing it to travel about 200 nm in the solution. If acceptor beads are within this distance, thioxene derivatives coated on the acceptor beads will accept the energy from singlet 02 and emit light at 520-620 nm (ALPHAScreen) or at 615 nm (ALPHALISA). The chance that acceptor beads are in proximity to donor beads would be increased significantly when binding partners A and B interact, and the tighter the binding, the higher the luminescence signal produced by the acceptor beads. As such, the interaction between A and B can be quantified by detecting the luminescence signal. A competitor that competes with A in binding to B would decrease the luminescence signal in a concentration-dependent manner, providing a competitive assay for the screening of inhibitors. PerkinElmer® (MA, USA) has made ALPHA very practical and easy by commercializing numerous ALPHA reagents. Meanwhile, the high signal-to-background ratio, high dynamic range, high sensitivity and wash-free procedure associated with the ALPHA makes this technology suitable for HTS application, allowing for the discovery of hits from a big library of compounds. In addition to the application of screening enzyme or protein inhibitors, the ALPHA has also been utilized to quantify the concentrations of proteins, to capture PPI, and to characterize ternary complexes formed between a target protein, a PROTAC degrader and an E3 ligase. Detecting ternary complex formation is one of the most useful applications of the ALPHA for PROTAC degraders. By titrating a PROTAC degrader to its target protein and E3 ligase, a bell-shaped curve can be produced when plotting ALPHA signals against the concentrations of the PROTAC. The height of the bell-shaped curve reflects the relative population of the ternary complex, allowing scientists to rank PROTAC degraders according to their ability to form ternary complexes.

Time-Resolved Fluorescence Energy Transfer Assay

Similar to the ALPHA, the time-resolved fluorescence energy transfer (TR-FRET) assay is also a proximity-based technology. It combines time-resolved fluorimetry with Forster resonance energy transfer (FRET), resulting in an assay that allows for very sensitive detection of binding/dissociation events in a homogeneous format. While the ALPHA uses a singlet oxygen transferred chemiluminescence signal to measure binding between two partners, the TR-FRET assay utilizes long-lived fluorophores combined with time-gated fluorescence intensity measurements to quantitate molecular association or dissociation events. In this assay, one binding partner is labeled with a donor fluorophore (usually a europium or terbium chelate/cryptate); the other binding partner is conjugated to a corresponding acceptor fluorophore. Europium and terbium are both lanthanides that have a long fluorescence life-time, large Stokes shift and a narrow emission peak. With these properties, europium and terbium can emit the fluorescent signals after the delay time when background fluorescence has decayed. In addition, the acceptor fluorophores used in the TR-FRET assay are chosen to have excitation wavelengths that are overlapped with the emission wavelengths of their paired donor fluorophores, allowing the energy transfer from donor fluorophores to acceptor fluorophores when they are in proximity. By detecting the emitted fluorescence signal of both the acceptor fluorophore and the donor fluorophore, the TR-FRET signal can be determined through a ratiometric or delta method, which is used to quantify the binding events of two binding partners that are conjugated to donor and acceptor fluorophores, respectively.

By designing the TR-FRET assay in different formats, this assay can be applied to perform HTS on small-molecule libraries, to quantify protein concentrations, to screen PPI targeting small molecules, as well as to characterize the ternary complex for PROTACs. Similar to the ALPHA, the ternary complex formation assay performed with TR-FRET also results in a bell-shaped curve, whose peak height reflects the relative population of the ternary complex. Compared with the ALPHA that captures binding partner to beads, the TR-FRET assay labels binding partner with fluorescent molecules, which are much smaller than beads, to provide more entropic freedom.

Isothermal Titration Calorimetry

Isothermal titration calorimetry (ITC) has been the gold standard for direct binding measurement. It measures the generation or consumption of heat following the titration of a ligand solution into a protein solution (or the reverse) to determine parameters including stoichiometry, Kd. changes in enthalpy (ΔH), changes in entropy (ΔS) and the heat capacity change (ΔCP). In each ITC experiment, stepwise injections of one reagent into a calorimetric cell containing the second reagent are performed and the exothermic or endothermic process of each injection is monitored. Analysis of the reaction mixture heat as a function of the analyte concentration provides a complete thermodynamic characterization of a binding event.

ITC is superior to any other biophysical techniques in that it can determine all binding parameters including stoichiometry (n), Kd. ΔH and ΔS in a single experiment. When in combination with structural information, ITC data can provide deeper insights into the mechanisms of binding, the binding driving force and the structure-function relationships [64]. ITC is also a true label-free biophysical method that allows the binding affinities to be determined without the need to tag or immobilize the binding partner, which is advantageous as tagging or immobilizing an analyte is sometimes technically difficult and may even interfere with the binding. In addition, the size of a molecule is not an issue for the ITC assay, whereas the sensitivity of other biophysical techniques such as SPR and bio-layer interferometry (BLI) is largely dependent on the molecular weight of their analytes. ITC has been increasingly used in determining protein-protein, protein-nucleic acid and protein-small molecule interactions. In recent years, ITC has also been applied in profiling the thermodynamic parameters and cooperativities of PROTAC molecules, providing invaluable insights into the interplay between a PROTAC molecule and its target protein and E3 ligase.

The sensitivity and accuracy of ITC instruments are improving with time. Two of the most commonly used, highly sensitive isothermal titration calorimeters are ITC-200 from Malvern Instrument (previously MicroCal, Malvern, UK) and Nano-ITC from TA Instrument (DE, USA).

SPR/BLI Assay

Both SPR and BLI are biophysical techniques that detect binding events through a spectroscopic method; the resulting sensorgrams show the kinetics of binding in real-time. The SPR assay is based on the changes in the refractive index of the medium directly in contact with the sensor chip surface, whereas BLI detects the changes in the interference pattern on the biosensor tip surface. Both SPR and BLI are label-free biophysical methods for determining interactions between ligands and targets, although they both require immobilization of one binding partner on the sensor chips/tips. The immobilization strategies for SPR and BLI are the same, but the availability of the corresponding modified sensor chips/tips is instrument dependent. The two broad approaches for performing the immobilization are chemical coupling and capture. Chemical coupling strategy results in covalent bonds between the ligands and the sensor chips/tips surface, while the capture strategy takes advantage of the strong affinity between the chemistry of the sensor chips/tips and the tag of the ligand.

In recent years, biosensor technology also finds its application in PROTACs, not only for binary binding determination but also for ternary complex characterization. In this aspect, SPR is more advantageous than ITC and other assays, as it not only provides ternary binding affinity (Kd) but also kinetics of a ternary complex.

Nano-BRET Assay

Nano-BRET is an optimized BRET technology using Nanoluc luciferase coupled with its substrate furimazine as the donor system [87]. The optimum properties of the Nanoluc and furimazine combination, such as high physical stability, high luminescence signal and small size, make Nano-BRET advantageous over previous BRET technologies and the combination of Nanoluc with various fluorescent protein acceptors greatly expands the application of BRET. The recent introduction of the Halo-Tag system as an alternative to fluorescent proteins offers an opportunity for multiplexing as fluorophores can be chosen as needed as long as chloroalkane-fluorophore conjugates are available [91]. In this new Nano-BRET system, one binding partner (e.g., protein A) is fused to Nanoluc and the other binding partner (e.g., protein B) is fused to Halo-Tag. The substrate of Nanoluc (furimazine) and ligand of Halo-Tag (chloroalkane-fluorophore conjugate) are added separately to the cells. Once in the cell, furimazine can be converted to furimamide by luciferase in the presence of oxygen and emit a luminescence signal at around 460 nm, whereas the chloroalkane-fluorophore conjugate attaches to Halo-Tag covalently and can be excited at 460 nm and emit light at around 618 nm. Interaction between protein A and B brings luciferase and Halo-Tag in proximity, allowing the luminescence signal to be transferred to a fluorophore (FIG. 14). With proper modification and adaptation, Nano-BRET technology can be applied to almost every step along the degradation pathway of PROTAC within live cells: from PROTAC target engagement, ternary complex formation and target ubiquitination to target degradation and target protein level detection. Nano-BRET can also be used to explain the mechanism of action of PROTAC molecules.

Nano-BRET is a relatively new technology developed and commercialized by Promega (WI, USA). It offers several advantages over other methods in profiling PROTAC molecules: Nano-BRET assay is carried out within live cells, and thus the results are based on physiological conditions; Nano-BRET uses endogenous protein in live cells, thus avoiding protein expression and purification process, which is favorable for target proteins that are hard to obtain or are in a large protein complex; the Nano-BRET assay can kinetically monitor target engagement and ternary complex formation, as well as target ubiquitination and degradation in real-time, providing more insights into the mechanism of action of PROTAC molecules.

Other Characterization of Bivalent Compounds

Assays other than the aforementioned that have also been used to characterize ternary complexes include size exclusive chromatography (SEC), crystallography, co-immunoprecipitation (Co-IP), mass spectrometry (MS) and the NanoBiT® assay.

The SEC ternary complex formation assay is based on the size difference between the binary complex and the ternary complex. By detecting the elution volume and elution time of proteins following PROTAC treatment, one can compare the relative ternary complex formation among different PROTAC molecules.

Crystallography is a challenging yet very useful biophysical technique. The ternary complex crystal structures of PROTAC degraders can provide valuable insights into how they bind to their target protein and E3 ligase, which can provide better guidance on the rational design and optimization of new degraders.

Co-IP can be used to detect PPI in live cells by using target protein-specific antibodies to indirectly capture proteins that are bound to a specific target protein. The enhanced capture in the presence of a PROTAC is an indication of ternary complex formation. The advantage of co-IP over other ternary complex formation assays is that it is most related to physiological conditions.

MS is another label-free technique that can provide insights into PROTAC degrader-mediated PPI. In some instances, native MS can preferentially reveal the E3-PROTAC-POI ternary complex in competition experiments with multiple substrate proteins present, thereby suggesting that it is not only an ideal HTS strategy for the development of new PROTACs but also a valuable tool to dissect the mechanism of actions, selectivity and specificity of PROTACs.

NanoBiT is a protein-fragment complementation assay that can detect PPI and ternary complex formation in live cells. In this assay, one binding partner is fused to a LgBiT (18 kDa) subunit, while the other binding partner is fused to a small BiT (SmBiT; 11 amino acid peptide). SmBiT is a complementary peptide of LgBiT that is designed to have a very low binding affinity to LgBiT. However, once SmBiT and LgBiT interact, they form an active enzyme that can generate a bright luminescent signal in the presence of a substrate. Interaction between LgBiT-fused and SmBiT-fused binding partners brings these two complementary proteins together, resulting in a luminescent signal as a measurement of PPI between binding partner proteins.

The thermal shift assay (TSA), also known as differential scanning calorimetry (DSF), studies thermal stabilization of proteins upon ligand binding. Given its capability to screen ligands that occupy the nonactive sites of proteins, DSF assay could be the potential assay to offer nonfunctional binding ligands for PROTAC design. By applying the TSA in a cellular formate, the cellular TSA (CETSA) allows studies of target engagement of PROTACs in a cellular context. The CETSA involves treatment of cells with a PROTAC of interest, heating to denature and precipitate proteins, cell lysis and the separation of cell debris and aggregates from the soluble protein fraction.

### Protein Ubiquitination Assays of Bivalent Compounds

Poly-ubiquitination is often the defining step in triggering target protein degradation. Carried out by the ubiquitin proteasome system, protein ubiquitination involves concerted actions of E1, E2 and E3 enzymes to attach ubiquitins to lysine residues of a target protein and subsequent ubiquitin chain elongation. The ubiquitinated proteins can be recognized, recruited and degraded by the 26S proteasome, a very large multicatalytic protease complex that breaks down ubiquitinated proteins to small peptides.

Ubiquitination assays can be done both in a cell-free system and in live cells using either electrophoresis-based or nonelectrophoresis-based methods. Substrate protein ubiquitination can also be detected in vitro with nonelectrophoresis-based methods such as FP, ALPHA and TR-FRET assays. Nevertheless, all these in vitro ubiquitination assays require additives such as ATP and E1, E2 and E3 enzymes, which is quite complicated in setting up the assay conditions and are not physiologically related. Ubiquitination assays carried out in live cells, however, allows for ubiquitination detection under native conditions by taking advantage of their own ubiquitination machinery. Immunoprecipitation of substrate proteins followed by ubiquitin immunoblotting is the simplest and one of the most commonly used methods to detect protein ubiquitination and it is likely to capture the ubiquitinated target protein specifically. Alternatively, the tagged versions of a target protein and ubiquitin can be expressed in cells to facilitate the detection. More recently, NanoBRET technology was applied to monitor intracellular BET protein ubiquitination induced by PROTAC degraders. In this NanoBRET-based assay, HiBiT-BET protein, its complementary protein LgBiT, and the luminescence substrate furimazine were used as an energy donor system, while the Halo-Tag fused ubiquitin and haloalkane fluorophore were employed as the respective energy acceptor system. A PROTAC degrader that can induce the ubiquitination of BET protein can bring HiBiT-BET protein in proximity to HaloTag-fused ubiquitin. With the involvement of LgBiT, furimazine and haloalkane fluorophore, an energy transfer from donor to acceptor can be expected, resulting in a fluorescence signal that reflects the intensity of ubiquitination.

It has been well-established that different ubiquitination sites and ubiquitination types can determine the fates of an ubiquitinated target protein. MS is a powerful technology that can not only be used in detecting total protein ubiquitination but also allows identification of ubiquitination sites and ubiquitination types.

A label-free technology named nanopore makes real-time detection of protein ubiquitination possible, yet it has not been applied to PROTAC degraders. In contrast to current ubiquitination assays that only detect end-point ubiquitination, the nanopore is capable of monitoring the E1-E2-E3 ubiquitination cascade kinetically. The TUBE-ALPHALISA and the TUBE-DELFIA (dissociation-enhanced lanthanide fluoroimmunoassay) are also two of the assays that can potentially be applied for quantifying ubiquitinated proteins in cell lysates following PROTAC treatment. The ease of operation and plate-based format of these two assays make high-throughput ubiquitination screening possible.

### Target Degradation Assays of Bivalent Compounds

Protein degradation, also called proteolysis, is a process that results in the hydrolysis of one or more peptide bonds in a protein. The ubiquitin-proteasome pathway (UPP) and autophagy are two main pathways and machineries that mediate degradation of intracellular proteins, while extracellular proteins and some cell surface proteins are taken up by endocytosis and are degraded within lysosomes. Protein degradation induced by PROTAC degraders is anticipated through UPP. Given the unpredictable nature of PROTAC degraders in inducing target protein degradation, it is of great importance to introduce reliable degradation assays to ultimately evaluate if the designed PROTAC is a target protein degrader. Methods that have been used to detect intracellular protein levels post PROTAC treatment include western blot, capillary-based immunoassay, fluorescence or luminescence-based reporter assays and MS based-proteomics.

Immunoassays

Western blot is the most frequently used method to measure the relative target protein levels in cells. However, the western blot assay relies on specific and high-quality antibodies for protein detection and is not suitable to accurately quantify the protein levels. In addition, the low sensitivity and multistep procedures of western blot could bring artifacts to the results. A capillary electrophoresis immunoassay is simpler than western blot with less sample consumption, simpler procedures and a shorter analysis time. It has been applied to determine the levels of BTK and pirin protein after cells treated with respective PROTACs. However, similar to western blot, this method also depends on the specific interaction between an antibody and an antigen for protein detection, presenting as a semiquantitative technique for protein detection. Other antibody-based methods that have been developed to quantitate protein levels include ELISA and ALPHA. Both ELISA and ALPHA provide highly sensitive protein level detection with the ALPHA being wash-free, having a larger dynamic range and allowing protein detection homogeneously. However, neither the ELISA nor the ALPHA have been applied to PROTAC degraders for protein degradation.

Reporter Assays

Fluorescence- or luminescence-based reporter assays represent a rapid and sensitive method to measure protein degradation in situ. Similarly, the development of a luciferase-based HiBiT tagging system offered another reporter assay to monitor protein degradation. In this assay, a target protein is fused with HiBiT using CRISPR/Cas9 editing in cell lines stably expressing LgBiT which complements with HiBiT to form the luminescent NanoBiT luciferase. Post PROTAC treatment, degradation of a target protein is reflected in the loss of luminescence signal. One challenge in these fluorescence- and luminescence-based assays is background interference, which can potentially decrease the dynamic range and sensitivity of these assays. However, once properly set up, these reporter assays can offer robust phenotype-based HTS methods for PROTACs.

Mass Spectrometry

MS analysis is a method that offers sensitive protein detection and quantification without relying on antibodies or tags. Various proteomic approaches are available to understand the mechanism of action of PROTAC degraders. The quantitative approach uses synthetic stable isotope-labeled proteins which can precisely mimic their endogenous counterparts as the internal standards to quantify the corresponding target protein. A global proteomic analysis study is widely used to examine the abundance change of proteins post treatment of with PROTAC degraders, to validate the degradation selectivity of the PROTAC degrader, and to reveal any off-target effects. The activities of these off-targets are probably responsible for the observed molecular and phenotypic responses. By offering an antibody- and tag-free protein quantitative method, MS makes intact protein detection possible. Moreover, with its capability to investigate the causes and consequences of protein degradation in biological systems, we anticipate its increasing application to the study of PROTAC degraders.

### Pharmaceutical Compositions

In some aspects, the compositions and methods described herein include the manufacture and use of pharmaceutical compositions and medicaments that include one or more bivalent compounds as disclosed herein. Also included are the pharmaceutical compositions themselves.

In some aspects, the pH of the compositions disclosed herein can be adjusted with pharmaceutically acceptable acids, bases, or buffers to enhance the stability of the bivalent compound or its delivery form.

Pharmaceutical compositions typically include a pharmaceutically acceptable excipient, adjuvant, or vehicle. As used herein, the phrase “pharmaceutically acceptable” refers to molecular entities and compositions that are generally believed to be physiologically tolerable and do not typically produce an allergic or similar untoward reaction, such as gastric upset, dizziness and the like, when administered to a human. A pharmaceutically acceptable excipient, adjuvant, or vehicle is a substance that can be administered to a patient, together with a compound of the invention, and which does not compromise the pharmacological activity thereof and is nontoxic when administered in doses sufficient to deliver a therapeutic amount of the compound. Exemplary conventional nontoxic pharmaceutically acceptable excipients, adjuvants, and vehicles include, but not limited to, saline, solvents, dispersion media, coatings, antibacterial and antifungal agents, isotonic and absorption delaying agents, and the like, compatible with pharmaceutical administration.

In particular, pharmaceutically acceptable excipients, adjuvants, and vehicles that can be used in the pharmaceutical compositions of this invention include, but are not limited to, ion exchangers, alumina, aluminum stearate, lecithin, self-emulsifying drug delivery systems (SEDDS) such as d-α-tocopherol polyethylene glycol 1000 succinate, surfactants used in pharmaceutical dosage forms such as Tweens or other similar polymeric delivery matrices, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. Cyclodextrins such as α-, β-, and γ-cyclodextrin, may also be advantageously used to enhance delivery of compounds of the formulae described herein.

Depending on the dosage form selected to deliver the bivalent compounds disclosed herein, different pharmaceutically acceptable excipients, adjuvants, and vehicles may be used. In the case of tablets for oral use, pharmaceutically acceptable excipients, adjuvants, and vehicles may be used include lactose and corn starch. Lubricating agents, such as magnesium stearate, are also typically added. For oral administration in a capsule form, useful diluents include lactose and dried corn starch. When aqueous suspensions or emulsions are administered orally, the active ingredient may be suspended or dissolved in an oily phase is combined with emulsifying or suspending agents. If desired, certain sweetening, flavoring, or coloring agents can be added.

In some embodiments, the bivalent compounds disclosed herein are defined to include pharmaceutically acceptable derivatives or prodrugs thereof. A “pharmaceutically acceptable derivative” means any pharmaceutically acceptable salt, solvate, or prodrug, e.g., carbamate, ester, phosphate ester, salt of an ester, or other derivative of a compound or agent disclosed herein, which upon administration to a recipient is capable of providing (directly or indirectly) a compound described herein, or an active metabolite or residue thereof. Particularly favored derivatives and prodrugs are those that increase the bioavailability of the compounds disclosed herein when such compounds are administered to a subject (e.g., by allowing an orally administered compound to be more readily absorbed into the blood) or which enhance delivery of the parent compound to a biological compartment (e.g., the brain or lymphatic system) relative to the parent species. Preferred prodrugs include derivatives where a group that enhances aqueous solubility or active transport through the gut membrane is appended to the structure of formulae described herein. Such derivatives are recognizable to those skilled in the art without undue experimentation. Nevertheless, reference is made to the teaching of Burger's Medicinal Chemistry and Drug Discovery, 5^{th} Edition, Vol. 1: Principles and Practice, which is incorporated herein by reference to the extent of teaching such derivatives.

The bivalent compounds disclosed herein include pure enantiomers, mixtures of enantiomers, pure diastereoisomers, mixtures of diastereoisomers, diastereoisomeric racemates, mixtures of diastereoisomeric racemates and the meso-form and pharmaceutically acceptable salts, solvent complexes, morphological forms, or deuterated derivatives thereof.

In some aspects, the pharmaceutical compositions disclosed herein can include an effective amount of one or more bivalent compounds. The terms “effective amount” and “effective to treat,” as used herein, refer to an amount or a concentration of one or more compounds or a pharmaceutical composition described herein utilized for a period of time (including acute or chronic administration and periodic or continuous administration) that is effective within the context of its administration for causing an intended effect or physiological outcome (e.g., treatment or prevention of cell growth, cell proliferation, or cancer). In some aspects, pharmaceutical compositions can further include one or more additional compounds, drugs, or agents used for the treatment of cancer (e.g., conventional chemotherapeutic agents) in amounts effective for causing an intended effect or physiological outcome (e.g., treatment or prevention of cell growth, cell proliferation, or cancer).

### Administration of Pharmaceutical Compositions

The pharmaceutical compositions disclosed herein can be formulated or adapted for administration to a subject via any route, e.g., any route approved by the Food and Drug Administration (FDA). Exemplary methods are described in the FDA Data Standards Manual (DSM) (available at http://www.fda.gov/Drugs/DevelopmentApprovalProcess/FormsSubmissionRequirements/ElectronicSubmissions/DataStandardsManualmonographs). In particular, the pharmaceutical compositions can be formulated for and administered via oral, parenteral, or transdermal delivery. The term “parenteral” as used herein includes subcutaneous, intracutaneous, intravenous, intramuscular, intraperitoneal, intra-articular, intra-arterial, intrasynovial, intrasternal, intrathecal, intralesional, and intracranial injection or infusion techniques.

For example, pharmaceutical compositions of this invention can be orally administered in any orally acceptable dosage form including, but not limited to, capsules, tablets, emulsions and aqueous suspensions, dispersions and solutions.

For example, the pharmaceutical compositions of this invention can be administered by injection (e.g., as a solution or powder). Such compositions can be formulated according to techniques known in the art using suitable dispersing or wetting agents (such as, for example, Tween 80) and suspending agents. The sterile injectable preparation may also be a sterile injectable solution or suspension in a non-toxic parenterally acceptable diluent or solvent, e.g., as a solution in 1,3-butanediol. Among the acceptable vehicles and solvents that may be employed are mannitol, water, Ringer's solution, and isotonic sodium chloride solution.

### Methods of Treatment

The methods disclosed herein contemplate administration of an effective amount of a compound or composition to achieve the desired or stated effect. In some aspects, provided herein are a bivalent compound described herein for treating or preventing one or more diseases or conditions disclosed herein in a subject in need thereof. In some aspects, provided herein are use of a bivalent compound in manufacture of a medicament for preventing or treating one or more diseases or conditions disclosed herein.

In some aspects, the methods disclosed include the administration of a therapeutically effective amount of one or more of the compounds or compositions described herein to a subject (e.g., a mammalian subject, e.g., a human subject). In some aspects, the methods disclosed include selecting a subject and administering to the subject an effective amount of one or more of the compounds or compositions described herein, and optionally repeating administration as required for the prevention or treatment of a disease or condition such as cancer.

In some aspects, subject selection can include obtaining a sample from a subject (e.g., a candidate subject) and testing the sample for an indication that the subject is suitable for selection. In some aspects, the subject can be confirmed or identified, e.g. by a health care professional, as having had, having an elevated risk to have, or having a condition or disease. In some aspects, suitable subjects include, for example, subjects who have or had a condition or disease but that resolved the disease or an aspect thereof, present reduced symptoms of disease (e.g., relative to other subjects (e.g., the majority of subjects) with the same condition or disease), or that survive for extended periods of time with the condition or disease (e.g., relative to other subjects (e.g., the majority of subjects) with the same condition or disease), e.g., in an asymptomatic state (e.g., relative to other subjects (e.g., the majority of subjects) with the same condition or disease). In some aspects, exhibition of a positive immune response towards a condition or disease can be made from patient records, family history, or detecting an indication of a positive immune response. In some aspects, multiple parties can be included in subject selection. For example, a first party can obtain a sample from a candidate subject and a second party can test the sample. In some aspects, subjects can be selected or referred by a medical practitioner (e.g., a general practitioner). In some aspects, subject selection can include obtaining a sample from a selected subject and storing the sample or using the in the methods disclosed herein. Samples can include, e.g., cells or populations of cells.

In some aspects, methods of treatment can include a single administration, multiple administrations, and repeating administration of one or more compounds disclosed herein as required for the prevention or treatment of the disease or condition disclosed herein (e.g., a POI-associated disease). In some aspects, methods of treatment can include assessing a level of disease in the subject prior to treatment, during treatment, or after treatment. In some aspects, treatment can continue until a decrease in the level of disease in the subject is detected.

The term “subject,” as used herein, refers to any animal. In some instances, the subject is a mammal. In some instances, the term “subject,” as used herein, refers to a human (e.g., a man, a woman, or a child).

The terms “administer,” “administering,” or “administration,” as used herein, refer to implanting, ingesting, injecting, inhaling, or otherwise absorbing a compound or composition, regardless of form. For example, the methods disclosed herein include administration of an effective amount of a compound or composition to achieve the desired or stated effect.

The terms “treat”, “treating,” or “treatment,” as used herein, refer to partially or completely alleviating, inhibiting, ameliorating, or relieving the disease or condition from which the subject is suffering. This means any manner in which one or more of the symptoms of a disease or disorder (e.g., cancer) are ameliorated or otherwise beneficially altered. As used herein, amelioration of the symptoms of a particular disorder (e.g., cancer) refers to any lessening, whether permanent or temporary, lasting or transient that can be attributed to or associated with treatment by the bivalent compounds, compositions and methods of the present invention.

The terms “prevent,” “preventing,” and “prevention,” as used herein, shall refer to a decrease in the occurrence of a disease or decrease in the risk of acquiring a disease or its associated symptoms in a subject. The prevention may be complete, e.g., the total absence of disease or pathological cells in a subject. The prevention may also be partial, such that the occurrence of the disease or pathological cells in a subject is less than, occurs later than, or develops more slowly than that which would have occurred without the present invention.

Specific dosage and treatment regimens for any particular patient will depend upon a variety of factors, including the activity of the specific compound employed, the age, body weight, general health status, sex, diet, time of administration, rate of excretion, drug combination, the severity and course of the disease, condition or symptoms, the patient's disposition to the disease, condition or symptoms, and the judgment of the treating physician.

An effective amount can be administered in one or more administrations, applications or dosages. A therapeutically effective amount of a therapeutic compound (i.e., an effective dosage) depends on the therapeutic compounds selected. Moreover, treatment of a subject with a therapeutically effective amount of the compounds or compositions described herein can include a single treatment or a series of treatments. For example, effective amounts can be administered at least once. The compositions can be administered from one or more times per day to one or more times per week; including once every other day. The skilled artisan will appreciate that certain factors can influence the dosage and timing required to effectively treat a subject, including but not limited to the severity of the disease or disorder, previous treatments, the general health or age of the subject, and other diseases present.

Following administration, the subject can be evaluated to detect, assess, or determine their level of disease. In some instances, treatment can continue until a change (e.g., reduction) in the level of disease in the subject is detected. Upon improvement of a patient's condition (e.g., a change (e.g., decrease) in the level of disease in the subject), a maintenance dose of a compound, or composition disclosed herein can be administered, if necessary. Subsequently, the dosage or frequency of administration, or both, can be reduced, e.g., as a function of the symptoms, to a level at which the improved condition is retained. Patients may, however, require intermittent treatment on a long-term basis upon any recurrence of disease symptoms.

### NON-LIMITING EMBODIMENTS

The present disclosure is also described by way of the following non-limiting embodiment. However, the use of these and other embodiments anywhere in the specification is illustrative only and in no way limits the scope and meaning of the disclosure. Likewise, the disclosure is not limited to any particular preferred embodiment or aspect described herein. Indeed, modifications and variations may be apparent to those skilled in the art upon reading this specification, and such variations can be made without departing from the disclosure in spirit or in scope.

1. A bivalent compound comprising a target binding moiety (TBM), and a cereblon E3 ubiquitin ligase binding moiety (CLM) represented by Formula (II)-1:

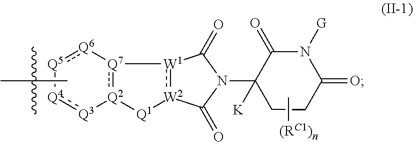

wherein:
W1 and W2 are each independently selected from C, CRC2 and N;
G is selected from the group consisting of H, OH, CH2OH, RC3OCOORC4, RC3OCONRC4RC5, and 2-(trimethylsilyl)ethoxymethyl group;
Q1 to Q7 are each independently C, O, S, N, CRC2 or NRC2; at least one of W1, W2, Q1, Q2, Q3, Q4, Q5, Q6 and Q7 comprises a heteroatom;
K is selected from the group consisting of H, an unsubstituted alkyl group, an alkyl group substituted by RC2, an unsubstituted cycloalkyl group, and a cycloalkyl group substituted by RC2; K is bound to the 6-membered ring with a stereospecific bond or a non-stereospecific bond;
RC1 is selected from the group consisting of an unsubstituted alkyl group, an alkyl group substituted by RC2, an unsubstituted aryl group, an aryl group substituted by RC2, an unsubstituted alkyl-aryl group, an alkyl-aryl group substituted by RC2, an unsubstituted alkoxy group, and an alkoxy group substituted by RC2;
RC2 is selected from the group consisting of H, halo, CH2OH, CRC4, NRC4RC5, 2-(trimethylsilyl)ethoxymethyl, an alkoxyl group, an unsubstituted alkyl group, an alkyl group substituted by one or more halo groups, an unsubstituted cycloalkyl group, a cycloalkyl group substituted by one or more halo groups, an unsubstituted aryl group, an aryl group substituted by one or more halo groups, an unsubstituted heteroaryl group, a heteroaryl group substituted by one or more halo groups, an unsubstituted heterocyclyl group, and a heterocyclyl group substituted by one or more halo groups;
RC3 is selected from the group consisting of an unsubstituted alkylene group, and an alkylene group substituted by RC2;
RC4 and RC5 are independently selected from the group consisting of an unsubstituted alkyl group, an alkyl group substituted by RC2, an unsubstituted cycloalkyl group, a cycloalkyl group substituted by RC2, an unsubstituted heterocyclyl group, a heterocyclyl group substituted by RC2, an unsubstituted aryl group, an aryl group substituted by RC2, an unsubstituted heteroaryl group, and a heteroaryl group substituted by RC2; and n is 0, 1, 2, 3 or 4, or a pharmaceutically acceptable salt or analog thereof, wherein the TBM is not selected from the group consisting of:

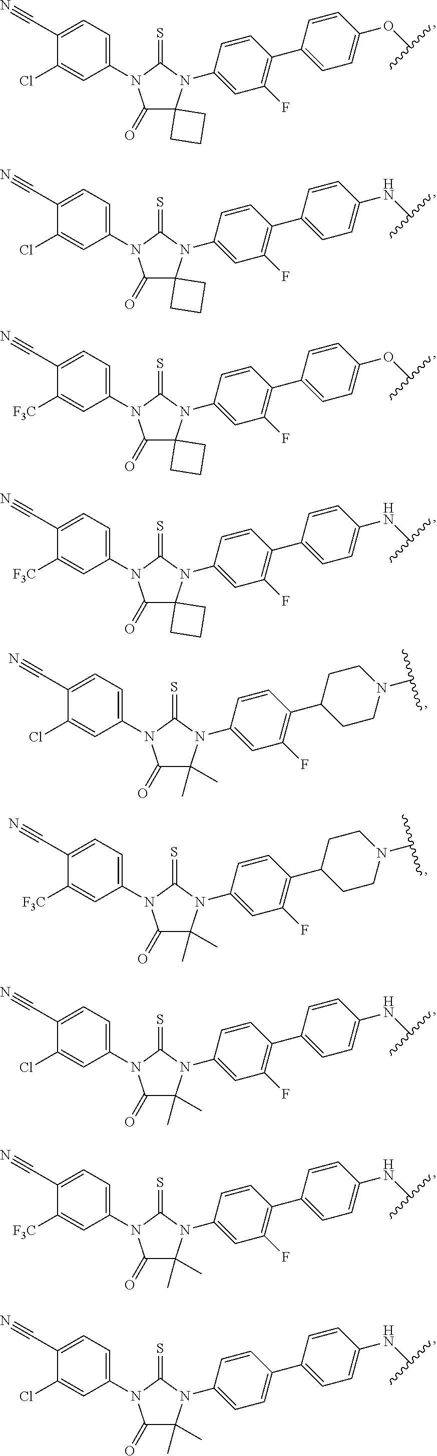

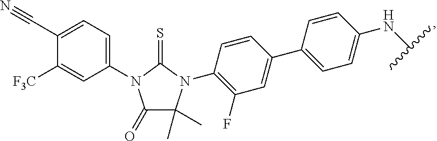 [SMILES: *Nc1ccc(-c2ccc(N3C(=S)N(c4ccc(C#N)c(C(F)(F)F)c4)C(=O)C3(C)C)c(F)c2)cc1]

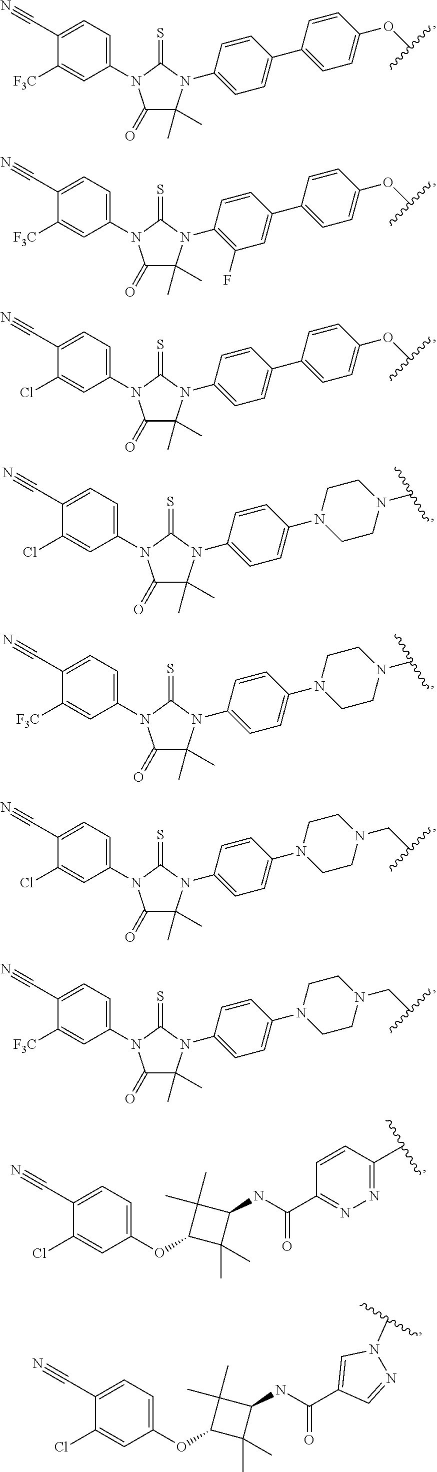

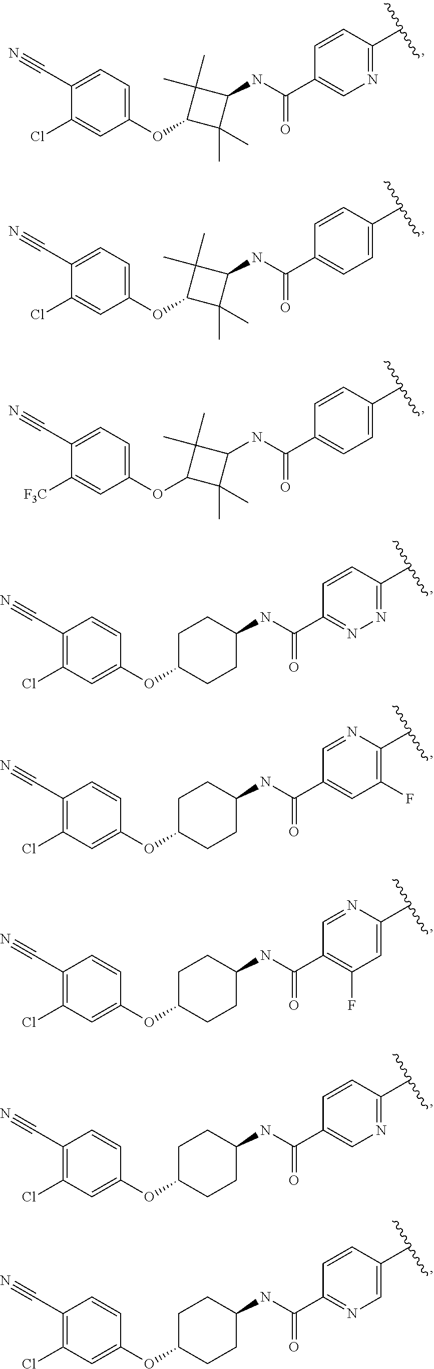

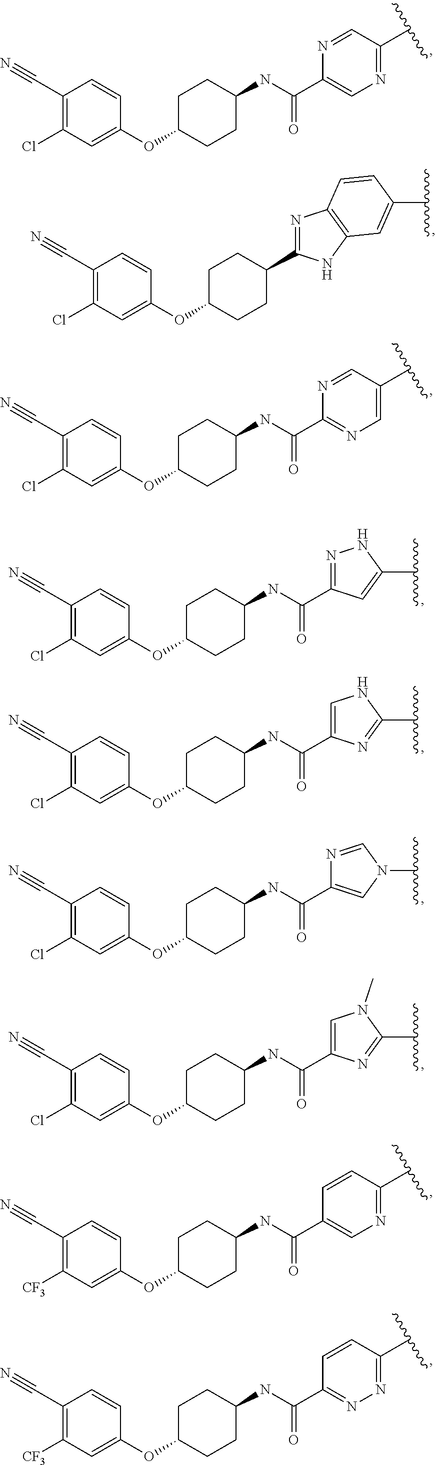

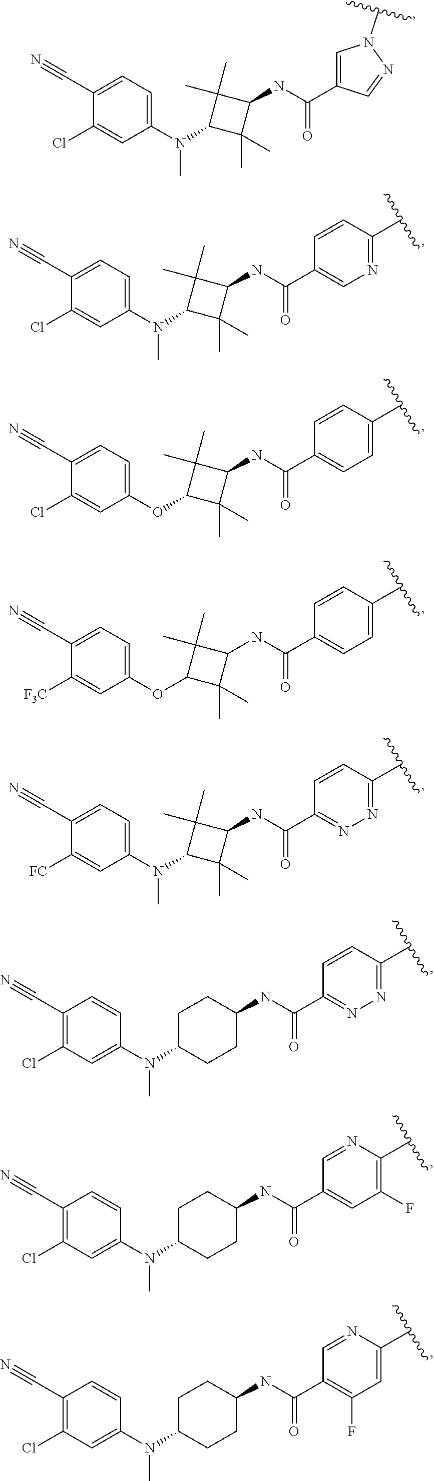

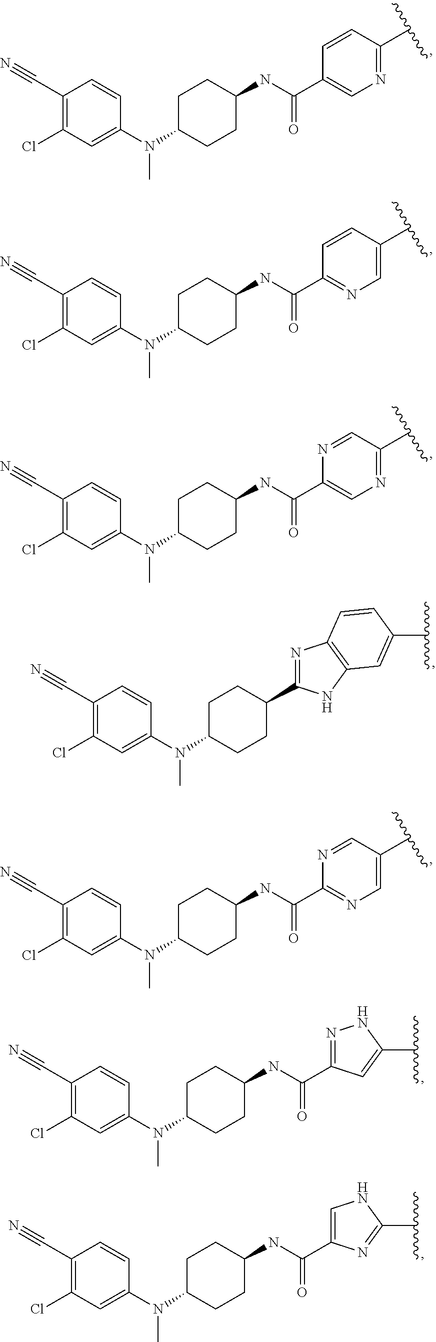

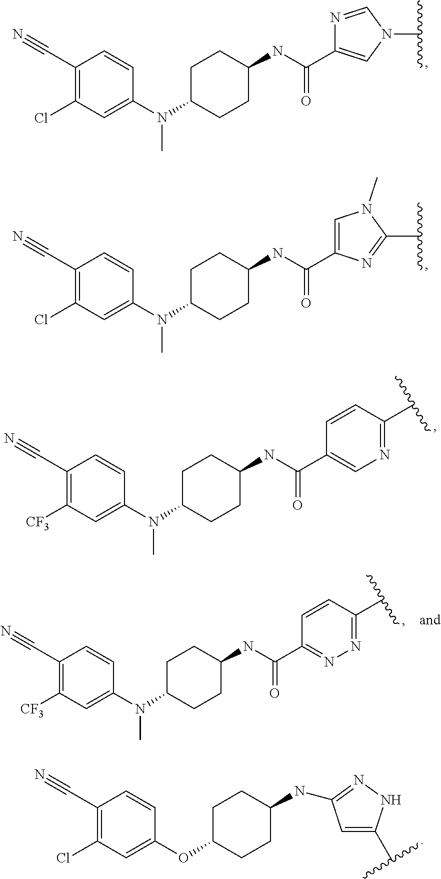 [SMILES: *n1cnc(C(=O)N[C@H]2CC[C@H](N(C)c3ccc(C#N)c(Cl)c3)CC2)c1 ; *c1nc(C(=O)N[C@H]2CC[C@H](N(C)c3ccc(C#N)c(Cl)c3)CC2)cn1C ; *c1ccc(C(=O)N[C@H]2CC[C@H](N(C)c3ccc(C#N)c(C(F)(F)F)c3)CC2)cn1 ; *c1ccc(C(=O)N[C@H]2CC[C@H](N(C)c3ccc(C#N)c(C(F)(F)F)c3)CC2)nn1 ; *c1cc(N[C@H]2CC[C@H](Oc3ccc(C#N)c(Cl)c3)CC2)n[nH]1]

2. The bivalent compound of embodiment 1, wherein the TBM is not selected from the group consisting of:

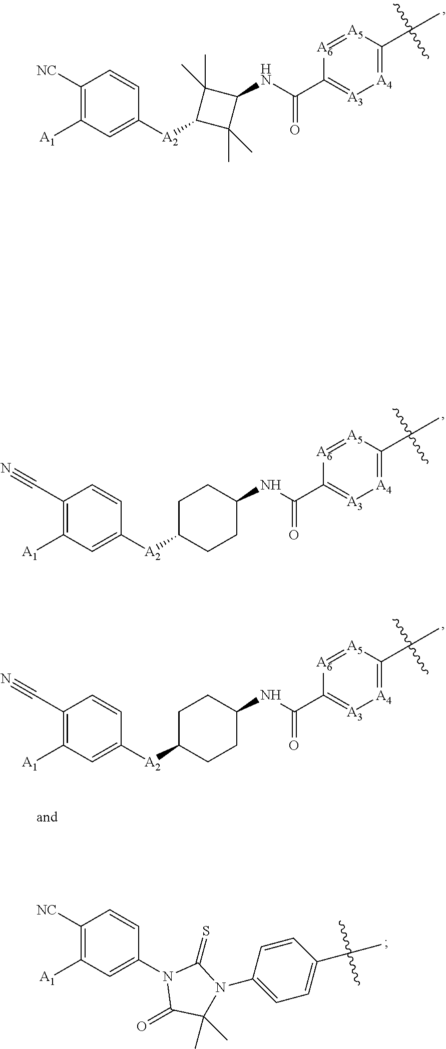

wherein A1 is selected from Cl, F, Br or CF3; A2 is selected from O, NH, N-methyl or N-ethyl; and A3, A4, A5 and A6 are each independently CH or N.
   3. The bivalent compound of embodiment 1, wherein the TBM is not selected from the group consisting of:

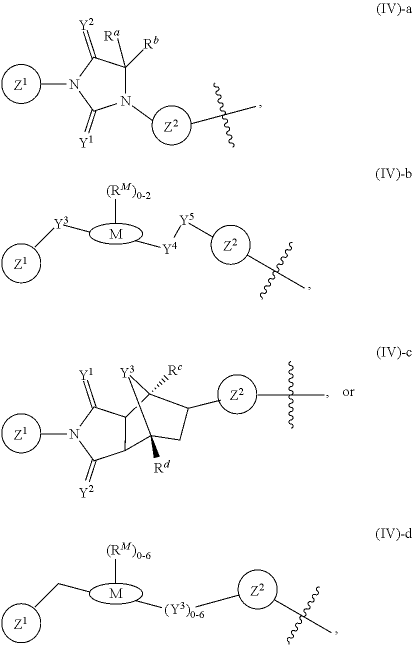

wherein Z1 is selected from the group consisting of an aryl group, a heteroaryl group, a bicyclic group, and a bi-heterocyclic group, each independently substituted by one or more substituents selected from the group consisting of a halo group, a hydroxyl group, a nitro group, CN, C≡CH, an unsubstituted C1-6 alkyl group, a C1-6 alkyl group substituted by a C1-6 alkoxyl group or one or more halo groups, an unsubstituted C1-6 alkoxyl group, a C1-6 alkoxyl group substituted by one or more halo groups, an unsubstituted C2-6 alkenyl, a C2-6 alkenyl substituted by one or more halo groups, an unsubstituted C2-6 alkynyl, and a C3-6 alkynyl substituted by one or more halo groups;
Y1, Y2, Y6 are each independently NRY1, O or S;
Y3, Y4, Y5 are each independently selected from the group consisting of a bond, O, NRY2, CRY1RY2, C═O, C═S, SO, SO2, a heteroaryl group, and an aryl group;
M is a 3- to 6-membered ring with 0 to 4 heteroatoms, which is unsubstituted or substituted by 1 to 6 RM groups;
each RM group is independently selected from the group consisting of H, an unsubstituted C1-6 alkyl group, a C1-6 alkyl group substituted by a C1-6 alkoxyl group or one or more halo groups, halogen, and a C1-6 alkoxy group; or two RM groups are taken together with the atom they are attached to and form a 3- to 8-membered ring system containing 0 to 2 heteroatoms;
Ra, Rb, Rc, Rd, RY1, RY2 are each independently selected from the group consisting of H, an unsubstituted C1-6 alkyl group, a C1-6 alkyl group substituted by a C1-6 alkoxyl group or one or more halo groups, halogen, a C1-6 alkoxy group, a cyclic group, and a heterocyclic group; or Ra, Rb are taken together with the atom they are attached to and form a 3- to 8-membered ring system containing 0 to 2 heteroatoms;
Z2 is selected from the group consisting of a bond, a C1-6 alkyl group, a C1-6 heteroalkyl group, 0, an aryl group, a heteroaryl group, an alicyclic group, a heterocyclic group, a biheterocyclic group, a biaryl group, and a biheteroaryl group, each of which is unsubstituted or substituted by 1 to 10 RZ2 groups;
each R^{Z2} group is independently selected from the group consisting of H, halo, an unsubstituted C1-6 alkyl group, a C1-6 alkyl group substituted by one or more F, —ORZ2A, a C3-6 cycloalkyl group, a C4-6 cycloheteroalkyl group, an unsubstituted C1-6 alkyl group, a C1-6 alkyl group substituted by a C1-3 alkyl group or a C1-6 alkoxyl group or one or more halo groups, an unsubstituted heterocyclic group, a heterocyclic group substituted by a C1-3 alkyl group or a C1-6 alkoxyl group or one or more halo groups, an unsubstituted aryl group, an aryl group substituted by a C1-3 alkyl group or a C1-6 alkoxyl group or one or more halo groups, an unsubstituted heteroaryl group, a heteroaryl group substituted by a C1-3 alkyl group or a C1-6 alkoxyl group or one or more halo groups, a bicyclic hereoaryl group, an unsubstituted OC1-3 alkyl group, and a OC1-3 alkyl substituted by one or more groups selected from F, OH, NH₂, NRY1RY2 and CN; and
R^{Z2A} is selected from the group consisting of H, a C1-6 alkyl group, and a C1-6 heteroalkyl group, each of which is unsubstituted or substituted by a cycloalkyl group, a cycloheteroalkyl group, an aryl group, a heterocyclic group, a heteroaryl group, halo, or a OC1-3 alkyl group.
   4. The bivalent compound of embodiment 1, wherein the TBM is capable of binding to a protein degradable by a cereblon E3 ubiquitin ligase.
   5. The bivalent compound of embodiment 1-4, wherein Q1 is NRC2.
   6. The bivalent compound of embodiment 5, wherein RC2 is an unsubstituted C1-6 alkyl group, a C1-6 alkyl group substituted by one or more halo groups, an unsubstituted C1-6 cycloalkyl group, a C1-6 cycloalkyl group substituted by one or more halo groups.
   7. The bivalent compound of embodiment 5, wherein RC2 is an unsubstituted C1-6 alkyl group.
   8. The bivalent compound of embodiment 5, wherein RC2 is an unsubstituted C1-4 alkyl group.
   9. The bivalent compound of embodiment 5, wherein RC2 is methyl or ethyl.
   10. The bivalent compound of embodiment 1-9, wherein W1 and W2 are each C and connected together through a double bond.
   11. The bivalent compound of embodiment 1-10, wherein Q2, Q3, Q4, Q5, Q6 and Q7 together form a benzene or heterobenzen ring, optionally substituted with one or more RC2.
   12. The bivalent compound of embodiment 1-10, wherein Q2, Q3, Q4, Q5, Q6 and Q7 together form an unsubstituted benzene or heterobenzen ring.
   13. The bivalent compound of embodiment 1-10, wherein Q2, Q3, Q4, Q5, Q6 and Q7 together form an unsubstituted benzene ring.
   14. The bivalent compound of embodiment 1-13, wherein K is H.
   15. The bivalent compound of embodiment 1-14, wherein n is 0, 1, or 2.
   16. The bivalent compound of embodiment 1-14, wherein n is 0 or 1.
   17. The bivalent compound of embodiment 1-14, wherein n is 0.
   18. The bivalent compound of embodiment 1-17, wherein G is selected from the group consisting of H, CH2OH, RC3OCOORC4, and RC3OCONRC4RC5.
   19. The bivalent compound of embodiment 1-17, wherein G is selected from the group consisting of H, CH2OH, and RC3OCOORC4.
   20. The bivalent compound of embodiment 1-17, wherein G is H.
   21. The bivalent compound of embodiment 1-17, wherein G is CH2OH.
   22. The bivalent compound of embodiment 1-17, wherein G is RC3OCOORC4.
   23. The bivalent compound of embodiment 22, wherein RC3 is selected from the group consisting of an unsubstituted alkylene group.
   24. The bivalent compound of embodiment 22, wherein RC3 is an unsubstituted C1-6 alkylene group.
   25. The bivalent compound of embodiment 22, wherein RC3 is an unsubstituted C1-4 alkylene group.
   26. The bivalent compound of embodiment 22, wherein RC3 is methylene or ethylene.
   27. The bivalent compound of embodiment 22, wherein RC3 is methylene.
   28. The bivalent compound of embodiment 22-27, wherein RC4 is an unsubstituted alkyl group or an alkyl group substituted by RC2.
   29. The bivalent compound of embodiment 22-27, wherein RC4 is an unsubstituted alkyl group.
   30. The bivalent compound of embodiment 22-27, wherein RC4 is an unsubstituted C1-6 alkyl group.
   31. The bivalent compound of embodiment 22-27, wherein RC4 is an unsubstituted C1-4 alkyl group.
   32. The bivalent compound of embodiment 22-27, wherein RC4 is methyl or ethyl.
   33. The bivalent compound of embodiment 22-27, wherein RC4 is methyl.
   34. The bivalent compound of embodiment 1-4, wherein the CLM is represented with Formula (II)-2:

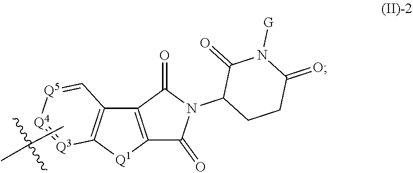

wherein
Q1 is O, S or NRC2;
Q3 to Q5 are each independently C or CRC2;
G is selected from the group consisting of H, OH, CH2OH, RC3OCOORC4 and 2-(trimethylsilyl)ethoxymethyl group;
RC2 is selected from the group consisting of H, CH2OH, CRC4, 2-(trimethylsilyl)ethoxymethyl, an unsubstituted C1-6 alkyl group, a C1-6 alkyl group substituted by one or more halo groups, a unsubstituted C1-6 cycloalkyl group, a C1-6 cycloalkyl group substituted by one or more halo groups;
RC3 is selected from the group consisting of a methylene group and a methylene group substituted by RC2;
RC4 is selected from the group consisting of an unsubstituted alkyl group, an alkyl group substituted by RC2, an unsubstituted cycloalkyl group, a cycloalkyl group substituted by RC2, an unsubstituted heterocyclyl group, a heterocyclyl group substituted by RC2.
   35. The bivalent compound of embodiment 34, wherein Q1 is NRC2.
   36. The bivalent compound of embodiment 35, wherein RC2 is an unsubstituted C1-6 alkyl group, a C1-6 alkyl group substituted by one or more halo groups, an unsubstituted C1-6 cycloalkyl group, a C1-6 cycloalkyl group substituted by one or more halo groups.
   37. The bivalent compound of embodiment 35, wherein RC2 is an unsubstituted C1-6 alkyl group.
   38. The bivalent compound of embodiment 35, wherein RC2 is an unsubstituted C1-4 alkyl group.
   39. The bivalent compound of embodiment 35, wherein RC2 is methyl or ethyl.
   40. The bivalent compound of embodiment 35, wherein Q3-Q5 are each C.
   41. The bivalent compound of embodiment 34-40, wherein G is selected from the group consisting of H, CH2OH, RC3OCOORC4, and RC3OCONRC4RC5.
   42. The bivalent compound of embodiment 34-40, wherein G is selected from the group consisting of H, CH2OH, and RC3OCOORC4.
   43. The bivalent compound of embodiment 34-40, wherein G is H.
   44. The bivalent compound of embodiment 34-40, wherein G is CH2OH.
   45. The bivalent compound of embodiment 34-40, wherein G is RC3OCOORC4.
   46. The bivalent compound of embodiment 45, wherein RC3 is selected from the group consisting of an unsubstituted alkylene group.
   47. The bivalent compound of embodiment 45, wherein RC3 is an unsubstituted C1-6 alkylene group.
   48. The bivalent compound of embodiment 45, wherein RC3 is an unsubstituted C1-4 alkylene group.
   49. The bivalent compound of embodiment 45, wherein RC3 is methylene or ethylene.
   50. The bivalent compound of embodiment 45, wherein RC3 is methylene.
   51. The bivalent compound of embodiment 45-50, wherein RC4 is an unsubstituted alkyl group or an alkyl group substituted by RC2.
   52. The bivalent compound of embodiment 45-50, wherein RC4 is an unsubstituted alkyl group.
   53. The bivalent compound of embodiment 45-50, wherein RC4 is an unsubstituted C1-6 alkyl group.
   54. The bivalent compound of embodiment 45-50, wherein RC4 is an unsubstituted C1-4 alkyl group.
   55. The bivalent compound of embodiment 45-50, wherein RC4 is methyl or ethyl.
   56. The bivalent compound of embodiment 45-50, wherein RC4 is methyl.
   57. The bivalent compound of embodiment 1-56, wherein the TBM is connected to the CLM through a bond or a linker moiety (L).
   58. The bivalent compound of embodiment 1-56, wherein the TBM is connected to the CLM through a linker moiety (L).
   59. The bivalent compound of embodiment 57-58, wherein the TBM is connected to the CLM through Q5.
   60. The bivalent compound of embodiment 57-58, wherein the TBM is connected to the CLM through Q4.
   61. The bivalent compound of embodiment 57-58, wherein the TBM is connected to the CLM through Q3.
   62. The bivalent compound of embodiment 57-61, wherein the linker moiety is of Formula (III):

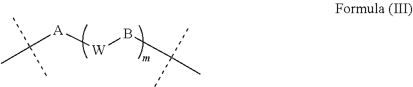

wherein
A, W, and B, at each occurrence, are independently selected from null, CO, CO₂, C(O)NR¹, C(S)NR¹, O, S, SO, SO₂, SO₂NR¹, NR¹, NR¹CO, NR¹CONR², NR¹C(S), optionally substituted C₁-C₈ alkyl, optionally substituted C₁-C₈ alkoxy, optionally substituted C₁-C₈alkoxyC₁-C₈alkyl, optionally substituted C₁-C₈ haloalkyl, optionally substituted C₁-C₈ hydroxyalkyl, optionally substituted C₂-C₈ alkenyl, optionally substituted C₂-C₈ alkynyl, optionally substituted C₃-C₈ cycloalkyl, optionally substituted C₃-C₈ cycloalkoxy, optionally substituted 3-8 membered heterocyclyl, optionally substituted aryl, optionally substituted heteroaryl, optionally substituted C₃-C₁₃ fused cycloalkyl, optionally substituted C₃-C₁₃ fused heterocyclyl, optionally substituted C₃-C₁₃ bridged cycloalkyl, optionally substituted C₃-C₁₃ bridged heterocyclyl, optionally substituted C₃-C₁₃ spiro cycloalkyl, and optionally substituted C₃-C₁₃ spiro heterocyclyl, wherein
   R¹ and R² are independently selected from hydrogen, optionally substituted C₁-C₆ alkyl, optionally substituted C₃-C₆ cycloalkyl, optionally substituted C₃-C₆ cycloalkoxy, optionally substituted 3-6 membered heterocyclyl, optionally substituted C₁-C₆ alkoxy, optionally substituted C₁-C₆ alkoxyalkyl, optionally substituted C₁-C₆ haloalkyl, optionally substituted C₁-C₆ hydroxyalkyl, optionally substituted C₁-C₆ alkylamino, and optionally substituted C₁-C₆alkylaminoC₁-C₆alkyl; and
m is 0 to 15.
   63. The bivalent compound of embodiment 57-61, wherein the linker moiety is of Formula (III)-1:

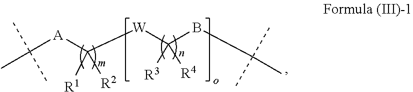

wherein
R¹, R², R³, and R⁴, at each occurrence, are independently selected from hydrogen, halogen, CN, OH, NH₂, optionally substituted C₁-C₆ alkyl, optionally substituted C₃-C₆ cycloalkyl, optionally substituted C₃-C₆ cycloalkoxy, optionally substituted 3-6 membered heterocyclyl, optionally substituted C₁-C₆ alkoxy, optionally substituted C₁-C₆ alkoxyalkyl, optionally substituted C₁-C₆ haloalkyl, optionally substituted C₁-C₆ hydroxyalkyl, optionally substituted C₁-C₆ alkylamino, and optionally substituted C₁-C₆alkylaminoC₁-C₆alkyl;
A, W, and B, at each occurrence, are independently selected from null, CO, CO₂, C(O)NR⁵, C(S)NR⁵, O, S, SO, SO₂, SO₂NR⁵, NR⁵, NR⁵CO, NR⁵CONR⁶, NR⁵C(S), optionally substituted C₁-C₈ alkyl, optionally substituted C₁-C₈ alkoxy, optionally substituted C₁-C₈alkoxyC₁-C₈alkyl, optionally substituted C₁-C₈ haloalkyl, optionally substituted C₁-C₈ hydroxyalkyl, optionally substituted C₂-C₈ alkenyl, optionally substituted C₂-C₈ alkynyl, optionally substituted C₃-C₈ cycloalkyl, optionally substituted C₃-C₈ cycloalkoxy, optionally substituted 3-8 membered heterocyclyl, optionally substituted aryl, optionally substituted heteroaryl, optionally substituted C₃-C₁₃ fused cycloalkyl, optionally substituted C₃-C₁₃ fused heterocyclyl, optionally substituted C₃-C₁₃ bridged cycloalkyl, optionally substituted C₃-C₁₃ bridged heterocyclyl, optionally substituted C₃-C₁₃ spiro cycloalkyl, and optionally substituted C₃-C₁₃ spiro heterocyclyl, wherein
   R⁵ and R⁶ are independently selected from hydrogen, optionally substituted C₁-C₆ alkyl, optionally substituted C₃-C₆ cycloalkyl, optionally substituted C₃-C₆ cycloalkoxy, optionally substituted 3-6 membered heterocyclyl, optionally substituted C₁-C₆ alkoxy, optionally substituted C₁-C₆ alkoxyalkyl, optionally substituted C₁-C₆ haloalkyl, optionally substituted C₁-C₆ hydroxyalkyl, optionally substituted C₁-C₆ alkylamino, and optionally substituted C₁-C₆alkylaminoC₁-C₆alkyl;
m is 0 to 15;
each n is 0 to 15; and
is 0 to 15.
   64. The bivalent compound of embodiment 57-61, wherein the linker moiety is of Formula (III)-2:

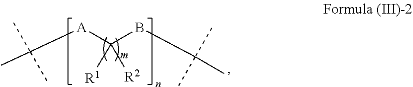

wherein
each R¹, and each R² are independently selected from hydrogen, halogen, CN, OH, NH₂, and optionally substituted C₁-C₆ alkyl, optionally substituted C₃-C₆ cycloalkyl, optionally substituted C₃-C₆ cycloalkoxy, optionally substituted 3-6 membered heterocyclyl, optionally substituted C₁-C₆ alkoxy, optionally substituted C₁-C₆ alkoxyalkyl, optionally substituted C₁-C₆ haloalkyl, optionally substituted C₁-C₆ hydroxyalkyl, optionally substituted C₁-C₆ alkylamino, or C₁-C₆alkylaminoC₁-C₆alkyl;
each A and each B are independently selected from null, CO, CO₂, C(O)NR³, C(S)NR³, O, S, SO, SO₂, SO₂NR³, NR³, NR³CO, NR³CONR⁴, NR³C(S), and optionally substituted C₁-C₈ alkyl, optionally substituted C₁-C₈ alkoxy, optionally substituted C₁-C₈alkoxyC₁-C₈alkyl, optionally substituted C₁-C₈ haloalkyl, optionally substituted C₁-C₈ hydroxyalkyl, optionally substituted C₂-C₈ alkenyl, optionally substituted C₂-C₈ alkynyl, optionally substituted C₃-C₈ cycloalkyl, optionally substituted C₃-C₈ cycloalkoxy, optionally substituted 3-8 membered heterocyclyl, optionally substituted aryl, optionally substituted heteroaryl, optionally substituted C₃-C₁₃ fused cycloalkyl, optionally substituted C₃-C₁₃ fused heterocyclyl, optionally substituted C₃-C₁₃ bridged cycloalkyl, optionally substituted C₃-C₁₃ bridged heterocyclyl, optionally substituted C₃-C₁₃ spiro cycloalkyl, or C₃-C₁₃ spiro heterocyclyl, wherein
R³ and R⁴ are independently selected from hydrogen, and optionally substituted C₁-C₆ alkyl, optionally substituted C₃-C₆ cycloalkyl, optionally substituted C₃-C₆ cycloalkoxy, optionally substituted 3-6 membered heterocyclyl, optionally substituted C₁-C₆ alkoxy, optionally substituted C₁-C₆ alkoxyalkyl, optionally substituted C₁-C₆ haloalkyl, optionally substituted C₁-C₆ hydroxyalkyl, optionally substituted C₁-C₆ alkylamino, or C₁-C₆alkylaminoC₁-C₆alkyl; each m is 0 to 15; and
n is 0 to 15.
   65. The bivalent compound of embodiment 57-61, wherein the linker moiety is of FORMULA (III)-3:

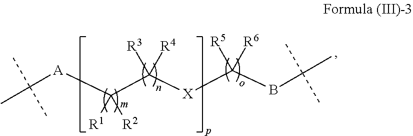

wherein
X is selected from O, NH, and NR⁷;
R¹, R², R³, R⁴, R⁵, and R⁶, at each occurrence, are independently selected from hydrogen, halogen, CN, OH, NH₂, optionally substituted C₁-C₆ alkyl, optionally substituted C₃-C₆ cycloalkyl, optionally substituted C₃-C₆ cycloalkoxy, optionally substituted 3-6 membered heterocyclyl, optionally substituted C₁-C₆ alkoxy, optionally substituted C₁-C₆ alkoxyalkyl, optionally substituted C₁-C₆ haloalkyl, optionally substituted C₁-C₆ hydroxyalkyl, optionally substituted C₁-C₆ alkylamino, and optionally substituted C₁-C₆alkylaminoC₁-C₆alkyl;
A and B, at each occurrence, are independently selected from null, CO, CO₂, C(O)NR⁷, C(S)NR⁷, O, S, SO, SO₂, SO₂NR⁷, NR⁷, NR⁷CO, NR⁷CONR⁸, NR⁷C(S), optionally substituted C₁-C₈ alkyl, optionally substituted C₁-C₈ alkoxy, optionally substituted C₁-C₈alkoxyC₁-C₈alkyl, optionally substituted C₁-C₈ haloalkyl, optionally substituted C₁-C₈ hydroxyalkyl, optionally substituted C₂-C₈ alkenyl, optionally substituted C₂-C₈ alkynyl, optionally substituted C₃-C₈ cycloalkyl, optionally substituted C₃-C₈ cycloalkoxy, optionally substituted 3-8 membered heterocyclyl, optionally substituted aryl, optionally substituted heteroaryl, optionally substituted C₃-C₁₃ fused cycloalkyl, optionally substituted C₃-C₁₃ fused heterocyclyl, optionally substituted C₃-C₁₃ bridged cycloalkyl, optionally substituted C₃-C₁₃ bridged heterocyclyl, optionally substituted C₃-C₁₃ spiro cycloalkyl, and optionally substituted C₃-C₁₃ spiro heterocyclyl, wherein
R⁸ and R⁸ are independently selected from hydrogen, optionally substituted C₁-C₆ alkyl, optionally substituted C₃-C₆ cycloalkyl, optionally substituted C₃-C₆ cycloalkoxy, optionally substituted 3-6 membered heterocyclyl, optionally substituted C₁-C₆ alkoxy, optionally substituted C₁-C₆ alkoxyalkyl, optionally substituted C₁-C₆ haloalkyl, optionally substituted C₁-C₆ hydroxyalkyl, optionally substituted C₁-C₆ alkylamino, and optionally substituted C₁-C₆alkylaminoC₁-C₆alkyl;
each m is 0 to 15;
each n is 0 to 15;
is 0 to 15; and
p is 0 to 15.
   66. The bivalent compound of embodiment 57-65, wherein the linker moiety comprises a 3 to 13 membered ring, a 4 to 13 membered fused ring, a 5 to 13 membered bridged ring, and a 5 to 13 membered spiro ring.
   67. The bivalent compound of embodiment 57-65, wherein the linker moiety comprises a ring selected from the group consisting of Formula C1, C2, C3, C4 and C5:

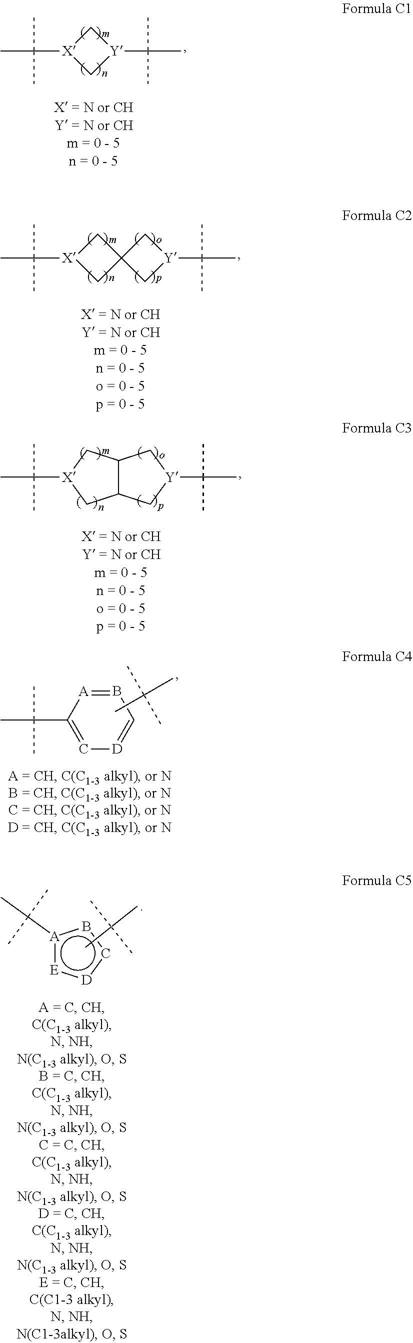

68. The bivalent compound of embodiment 57-61, wherein the linker moiety is of Formula (IV):

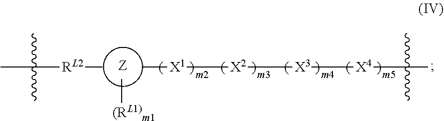

wherein
Z is selected from the group consisting of a 3- to 8-membered ring, a 5- to 12-membered bicyclic ring, an 8- to 15-membered tricyclic ring and a 6- to 12-membered spiro bicyclic ring, each independently having 0-4 heteroatoms;
RL1 is selected from the group consisting of H, an unsubstituted C1-6 alkyl group, a C1-6 alkyl group substituted by a C1-6 alkoxyl group or one or more halo groups, halogen, a C1-6 alkoxy group, a keto group, or an oxide group; wherein, two RL1 groups are optionally taken together to form a 3-8 membered ring system containing 0-2 heteroatoms; RL2 is a bond or an ethynylene group;
X1 is selected from the group consisting of a methylene group and an ethylene group;
X2 and X4 are independently selected from the group consisting of a methylene group, C(═O), C(═O)C(═O), CONRL3, NRL3C(═O), NRL3C(═O)NRL4, NRL3C(═O)C(═O)NRL4, NRL3SO2NRL4, SO2NRL3, CRL3RL4, NRL5, O and S;
X3 is selected from the group consisting of an unsubstituted C1-8 alkylene group, a C1-8 alkylene group substituted by 1 to 6 RL1, an unsubstituted C1-8 heteroalkylene group, a C1-8 heteroalkylene group substituted by 1 to 6 RL1, an unsubstituted 3- to 8-membered arylene group, a 3- to 8-membered arylene group substituted by 1 to 6 RL1, an unsubstituted 3- to 8-membered heteroarylene group with 1 to 3 hetero atoms, and a 3- to 8-membered heteroarylene group with 1 to 3 hetero atoms and substituted by 1 to 6 RL1, an unsubstituted 3- to 7-membered cyclic alkylene group, a 3- to 7-membered cyclic alkylene group substituted by 1 to 6 RL1, an unsubstituted 3- to 7-membered heterocyclic alkylene group with 1 to 2 hetero atoms, and a 3- to 7-membered heterocyclic alkylene group with 1 to 2 hetero atoms and substituted by 1 to 6 RL1; or, X3 is a 6- to 12-membered spiro bicyclic ring having 0-4 heteroatoms;
RL3 is hydrogen;
RL4 and RL5 are independently selected from the group consisting of H, an unsubstituted C1-6 alkyl group, and a C1-6 alkyl group substituted by a C1-6 alkoxyl group or one or more halo groups;
m1 is 0, 1, 2, 3, 4, 5 or 6;
m2 is 0 or 1;
m3 is 0 or 1;
m4 is 1; and
m5 is 0 or 1.
   69. The bivalent compound of embodiment 68, wherein the heteroatoms in Formula (IV) are each independently selected N, O and S.
   70. The bivalent compound of embodiment 68-69, wherein the Z ring comprises one or two heteroatoms selected from N, O and S.
   71. The bivalent compound of embodiment 68-70, wherein Z is selected from the group consisting of a 3- to 8-membered ring, a 6- to 10-membered bicyclic ring and a 8- to 10-membered spiro bicyclic ring, each independently having 1-2 heteroatoms.
   72. The bivalent compound of embodiment 68-71, wherein RL1 is selected from the group consisting of H, an unsubstituted linear C1-3 alkyl group, a keto group, or an oxide group;
   73. The bivalent compound of embodiment 68-72, wherein RL2 is a bond or an ethynylene group.
   74. The bivalent compound of embodiment 68-73, wherein X1 is selected from the group consisting of a methylene group and an ethylene.
   75. The bivalent compound of embodiment 68-74, wherein X2 is selected from the group consisting of C(═O), NRL3C(═O) and O.
   76. The bivalent compound of embodiment 68-75, wherein X3 is selected from the group consisting of an unsubstituted C1-6 alkylene group, an unsubstituted 3- to 7-membered cyclic alkylene group, and an unsubstituted 3- to 7-heterocyclic alkylene group with 0 to 2 hetero atoms; or, X3 is a 10- to 12-membered spiro bicyclic ring having 0-2 heteroatoms.
   77. The bivalent compound of embodiment 68-76, wherein X4 is selected from the group consisting of a methylene group, CONRL3 and O.
   78. The bivalent compound of embodiment 68-77, wherein m1 is 0, 1, 2 or 3.
   79. The bivalent compound of embodiment 68-78, wherein RL1 is selected from the group consisting of H, an unsubstituted C1-3 alkyl group, a C1-3 alkyl group substituted by a C1-3 alkoxyl group or one or more halo groups, halogen, a C1-3 alkoxy group, a keto group, or an oxide group; wherein, two RL1 groups are optionally taken together to form a 3-8 membered ring system containing 0-2 heteroatoms.
   80. The bivalent compound of embodiment 68-78, wherein RL1 is an oxide group which is attached to one heteroatom N on the Z ring to form an N-oxide group (N⁺—O⁻).
   81. The bivalent compound of embodiment 68-78, wherein RL1 is an alkyl selected from the group consisting of a linear C1-6 alkyl and a branched C1-6 alkyl, each of which is unsubstituted or substituted by a C1-6 alkoxyl group or one or more halo groups.
   82. The bivalent compound of embodiment 68-78, wherein RL1 is an alkyl selected from the group consisting of a linear C1-3 alkyl and a branched C1-3 alkyl, each of which is unsubstituted or substituted by a C1-6 alkoxyl group or one or more halo groups.
   83. The bivalent compound of embodiment 68-82, wherein X3 is selected from the group consisting of a bond, an unsubstituted C1-6 alkylene group, a C1-6 alkylene group substituted by 1 to 6 RL1, an unsubstituted C1-6 heteroalkylene group, a C1-6 heteroalkylene group substituted by 1 to 6 RL1.
   84. The bivalent compound of embodiment 68-82, wherein X3 is selected from the group consisting of an unsubstituted C1-5 alkylene group.
   85. The bivalent compound of embodiment 68-84, wherein RL4 and RL5 are independently an alkyl selected from the group consisting of a linear C1-6 alkyl and a branched C1-6 alkyl, each of which is unsubstituted or substituted by a C1-6 alkoxyl group or one or more halo groups.
   86. The bivalent compound of embodiment 68-84, wherein RL4 and RL5 are independently an alkyl selected from the group consisting of a linear C1-3 alkyl and a branched C1-3 alkyl, each of which is unsubstituted or substituted by a C1-6 alkoxyl group or one or more halo groups.
   87. The bivalent compound of embodiment 57-61, wherein the linker moiety is selected from the group consisting of:

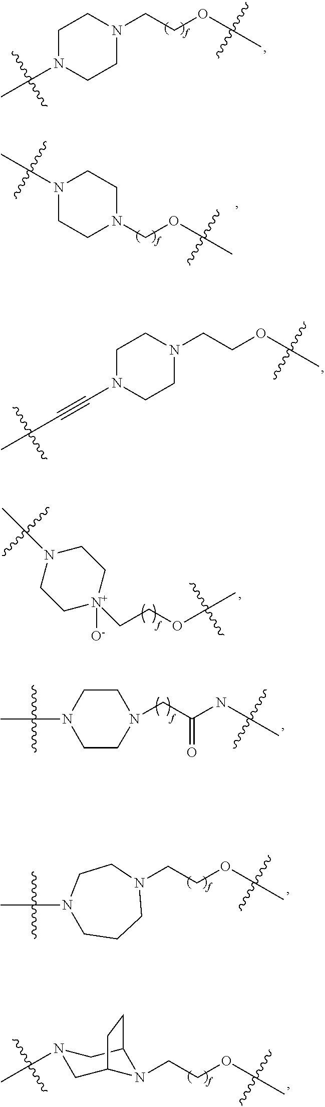

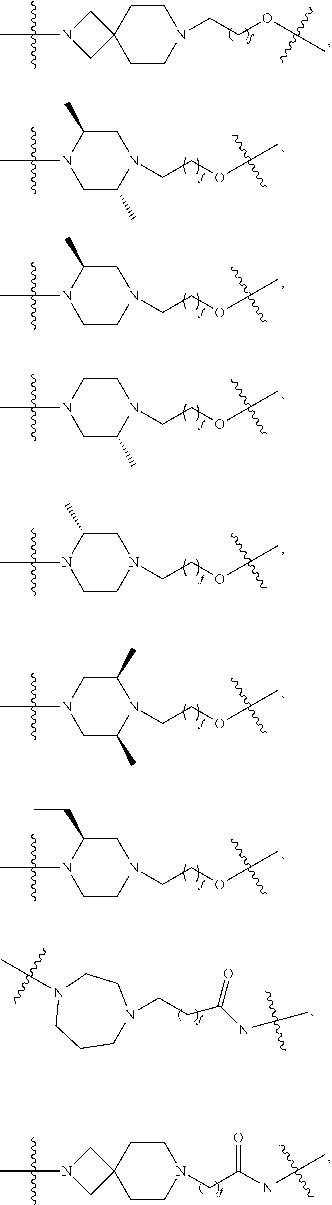

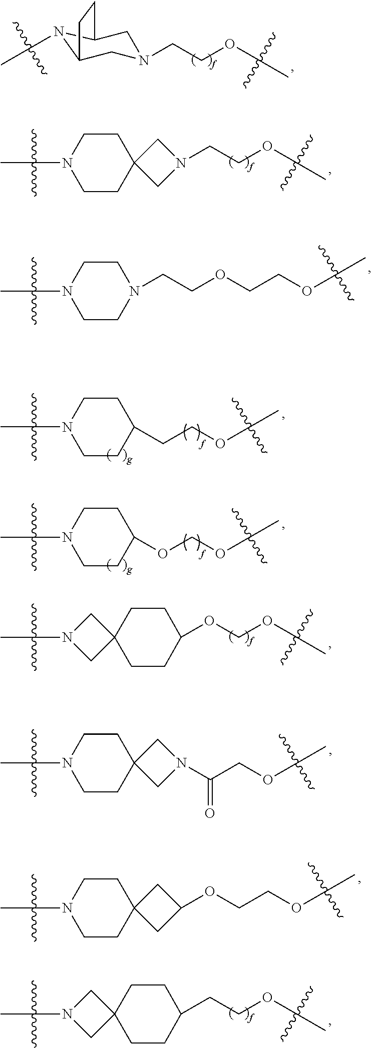

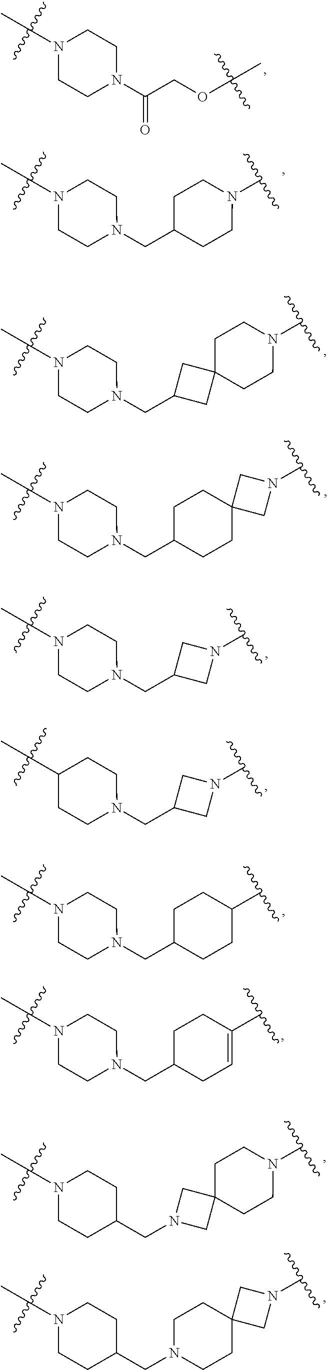

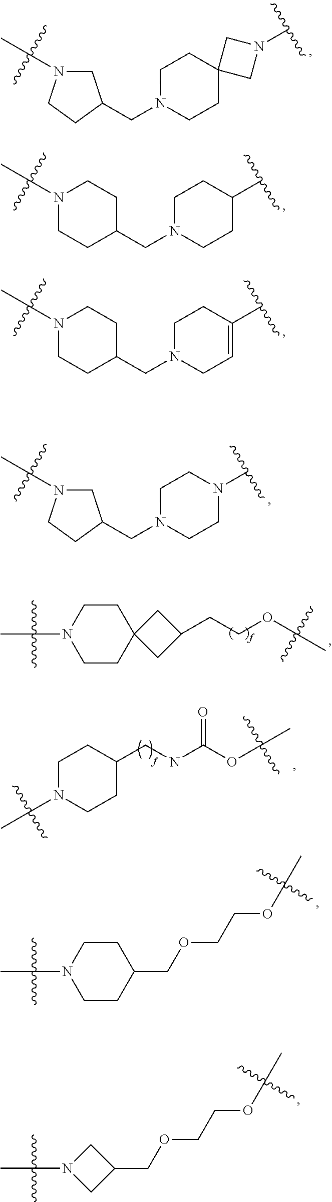

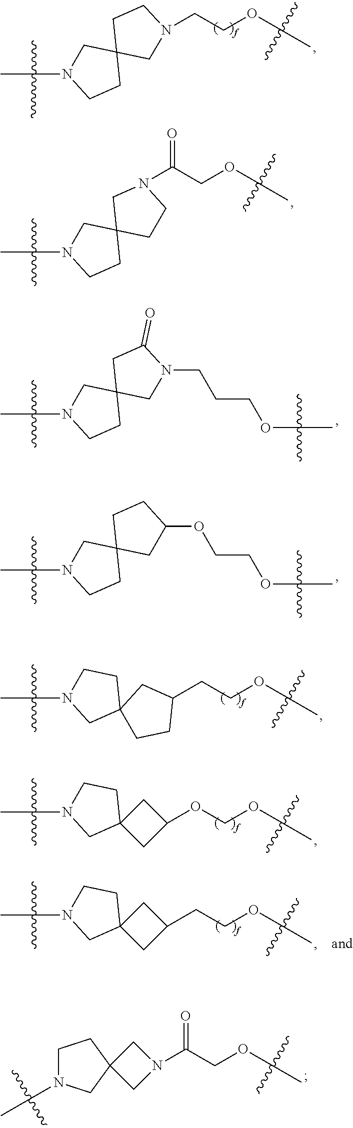

wherein f is an integer of 0, 1, 2, 3 or 4; and g is an integer of 0, 1, 2 or 3.
88. A bivalent compound selected from the group consisting of

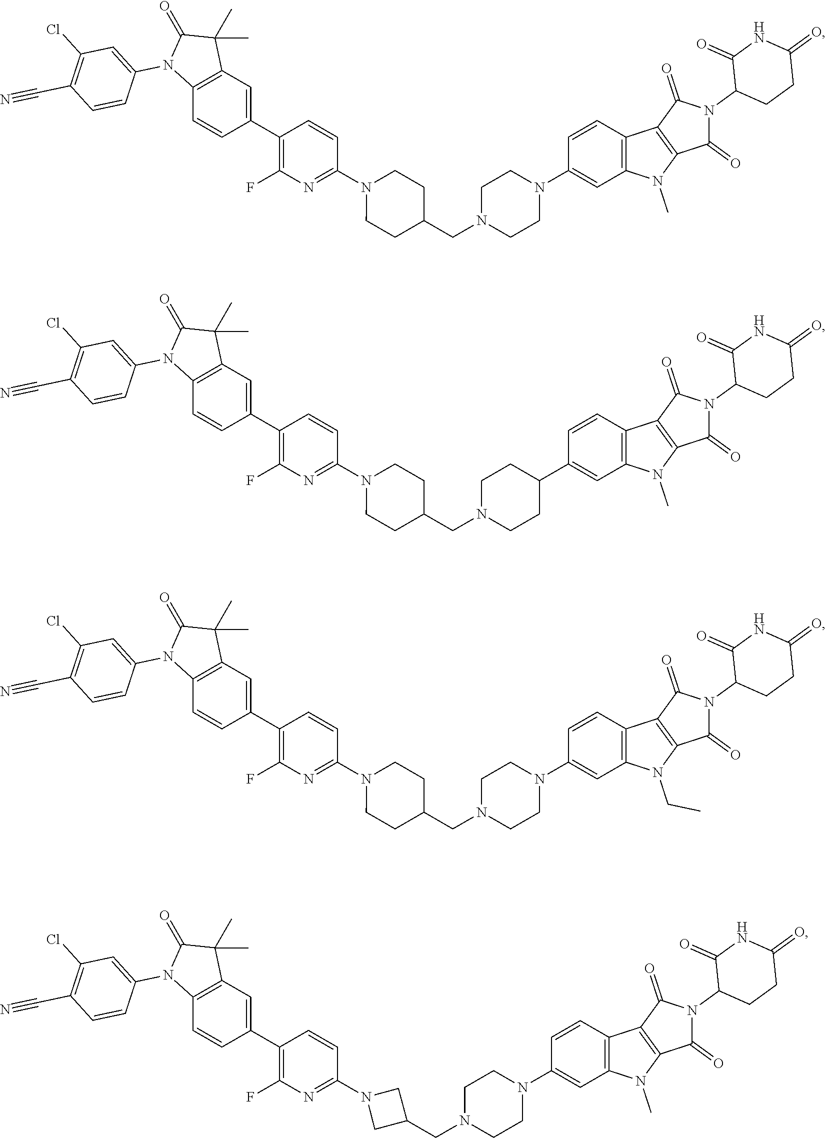 [SMILES: Cn1c2c(c3ccc(N4CCN(CC5CCN(c6ccc(-c7ccc8c(c7)C(C)(C)C(=O)N8c7ccc(C#N)c(Cl)c7)c(F)n6)CC5)CC4)cc31)C(=O)N(C1CCC(=O)NC1=O)C2=O ; Cn1c2c(c3ccc(C4CCN(CC5CCN(c6ccc(-c7ccc8c(c7)C(C)(C)C(=O)N8c7ccc(C#N)c(Cl)c7)c(F)n6)CC5)CC4)cc31)C(=O)N(C1CCC(=O)NC1=O)C2=O ; CCn1c2c(c3ccc(N4CCN(CC5CCN(c6ccc(-c7ccc8c(c7)C(C)(C)C(=O)N8c7ccc(C#N)c(Cl)c7)c(F)n6)CC5)CC4)cc31)C(=O)N(C1CCC(=O)NC1=O)C2=O ; Cn1c2c(c3ccc(N4CCN(CC5CN(c6ccc(-c7ccc8c(c7)C(C)(C)C(=O)N8c7ccc(C#N)c(Cl)c7)c(F)n6)C5)CC4)cc31)C(=O)N(C1CCC(=O)NC1=O)C2=O]

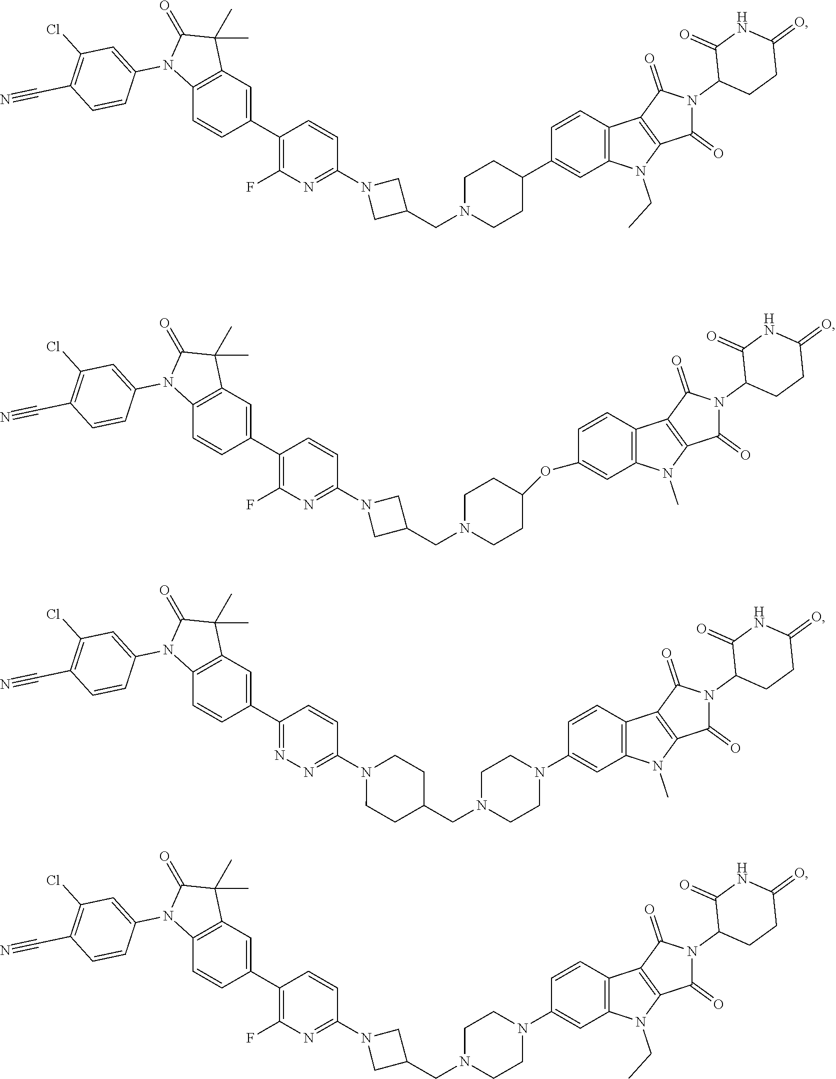 [SMILES: CCn1c2c(c3ccc(C4CCN(CC5CN(c6ccc(-c7ccc8c(c7)C(C)(C)C(=O)N8c7ccc(C#N)c(Cl)c7)c(F)n6)C5)CC4)cc31)C(=O)N(C1CCC(=O)NC1=O)C2=O ; Cn1c2c(c3ccc(OC4CCN(CC5CN(c6ccc(-c7ccc8c(c7)C(C)(C)C(=O)N8c7ccc(C#N)c(Cl)c7)c(F)n6)C5)CC4)cc31)C(=O)N(C1CCC(=O)NC1=O)C2=O ; Cn1c2c(c3ccc(N4CCN(CC5CCN(c6ccc(-c7ccc8c(c7)C(C)(C)C(=O)N8c7ccc(C#N)c(Cl)c7)nn6)CC5)CC4)cc31)C(=O)N(C1CCC(=O)NC1=O)C2=O ; CCn1c2c(c3ccc(N4CCN(CC5CN(c6ccc(-c7ccc8c(c7)C(C)(C)C(=O)N8c7ccc(C#N)c(Cl)c7)c(F)n6)C5)CC4)cc31)C(=O)N(C1CCC(=O)NC1=O)C2=O]

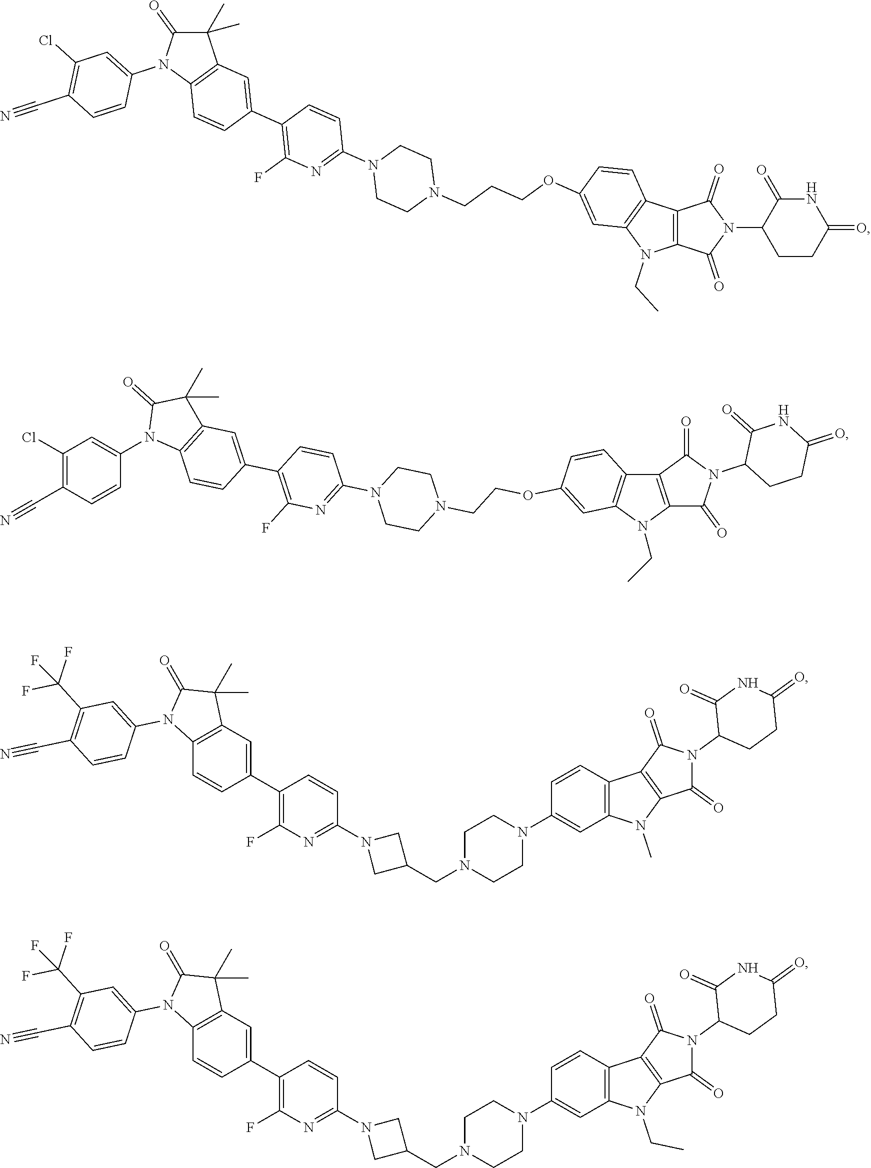 [SMILES: CCn1c2c(c3ccc(OCCCN4CCN(c5ccc(-c6ccc7c(c6)C(C)(C)C(=O)N7c6ccc(C#N)c(Cl)c6)c(F)n5)CC4)cc31)C(=O)N(C1CCC(=O)NC1=O)C2=O ; CCn1c2c(c3ccc(OCCN4CCN(c5ccc(-c6ccc7c(c6)C(C)(C)C(=O)N7c6ccc(C#N)c(Cl)c6)c(F)n5)CC4)cc31)C(=O)N(C1CCC(=O)NC1=O)C2=O ; Cn1c2c(c3ccc(N4CCN(CC5CN(c6ccc(-c7ccc8c(c7)C(C)(C)C(=O)N8c7ccc(C#N)c(C(F)(F)F)c7)c(F)n6)C5)CC4)cc31)C(=O)N(C1CCC(=O)NC1=O)C2=O ; CCn1c2c(c3ccc(N4CCN(CC5CN(c6ccc(-c7ccc8c(c7)C(C)(C)C(=O)N8c7ccc(C#N)c(C(F)(F)F)c7)c(F)n6)C5)CC4)cc31)C(=O)N(C1CCC(=O)NC1=O)C2=O]

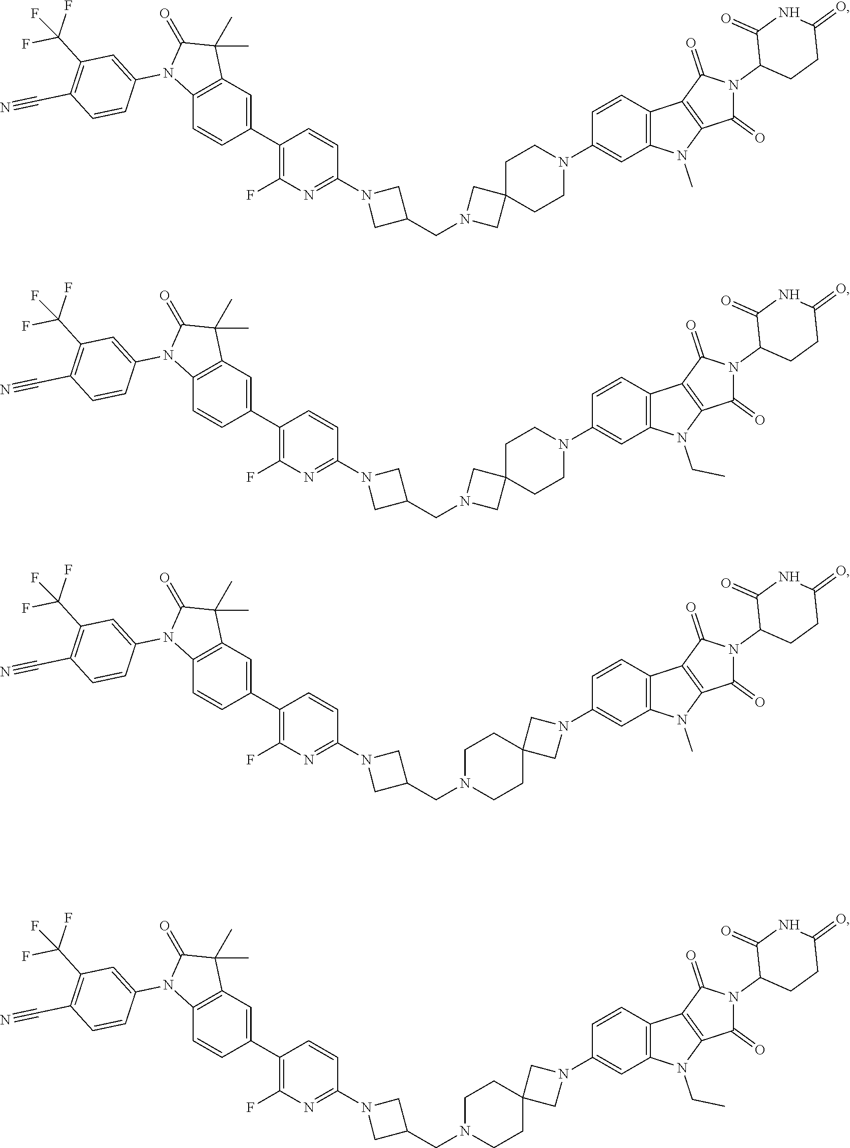

or pharmaceutically acceptable salts thereof.
89. A composition comprising a bivalent compound according to embodiment 1-88, and a pharmaceutically acceptable carrier.
90. A method of degrading a protein associated with a disease or condition, by contacting the protein with the bivalent compound according to embodiment 1-88.

### NON-LIMITING EXAMPLES

The present disclosure is also described and demonstrated by way of the following non-limiting examples. However, the use of these and other examples anywhere in the specification is illustrative only and in no way limits the scope and meaning of the disclosure or of any exemplified term. Likewise, the disclosure is not limited to any particular preferred embodiment or aspect described herein. Indeed, suitable modifications and variations may be apparent to those skilled in the art upon reading this specification, and such variations can be made without departing from the invention in spirit or in scope.

### Certain Examples of Bivalent Compounds

Shown in Table 1 below are exemplary of the bifunctional compounds, showing the Compound ID and structure:

TABLE 1

| ID | Formula |
|---|---|
| 1 | 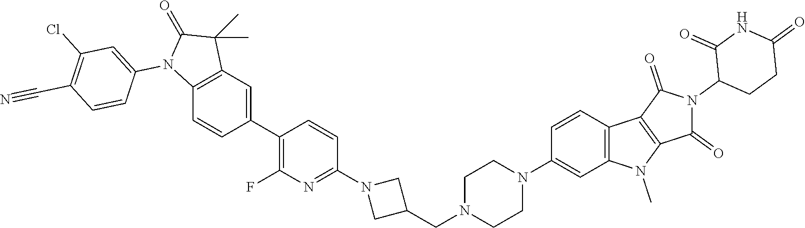 [SMILES: Cn1c2c(c3ccc(N4CCN(CC5CN(c6ccc(-c7ccc8c(c7)C(C)(C)C(=O)N8c7ccc(C#N)c(Cl)c7)c(F)n6)C5)CC4)cc31)C(=O)N(C1CCC(=O)NC1=O)C2=O] |
| 2 | 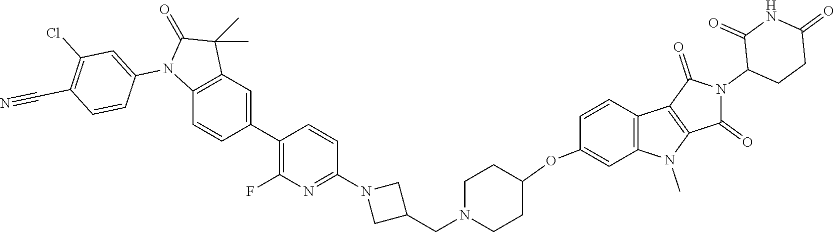 [SMILES: Cn1c2c(c3ccc(OC4CCN(CC5CN(c6ccc(-c7ccc8c(c7)C(C)(C)C(=O)N8c7ccc(C#N)c(Cl)c7)c(F)n6)C5)CC4)cc31)C(=O)N(C1CCC(=O)NC1=O)C2=O] |
| 3 | 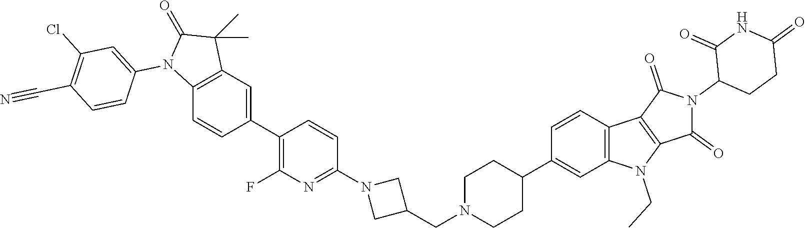 [SMILES: CCn1c2c(c3ccc(C4CCN(CC5CN(c6ccc(-c7ccc8c(c7)C(C)(C)C(=O)N8c7ccc(C#N)c(Cl)c7)c(F)n6)C5)CC4)cc31)C(=O)N(C1CCC(=O)NC1=O)C2=O] |
| 4 | 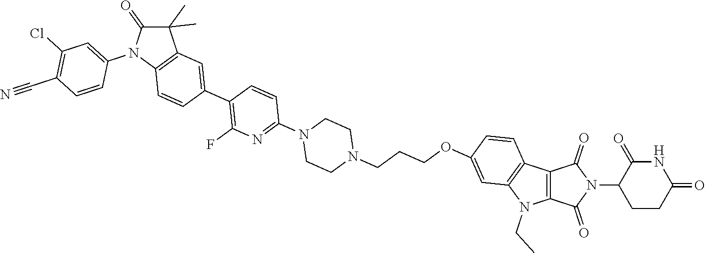 [SMILES: CCn1c2c(c3ccc(OCCCN4CCN(c5ccc(-c6ccc7c(c6)C(C)(C)C(=O)N7c6ccc(C#N)c(Cl)c6)c(F)n5)CC4)cc31)C(=O)N(C1CCC(=O)NC1=O)C2=O] |
| 5 | 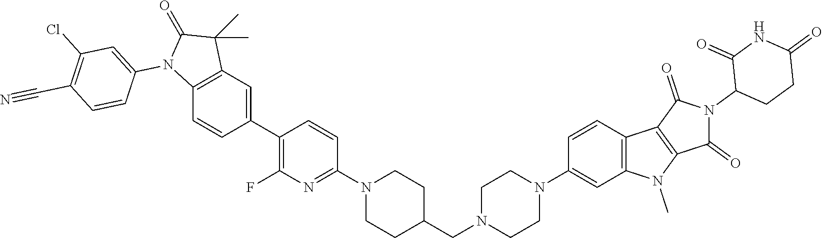 [SMILES: Cn1c2c(c3ccc(N4CCN(CC5CCN(c6ccc(-c7ccc8c(c7)C(C)(C)C(=O)N8c7ccc(C#N)c(Cl)c7)c(F)n6)CC5)CC4)cc31)C(=O)N(C1CCC(=O)NC1=O)C2=O] |
| 6 | 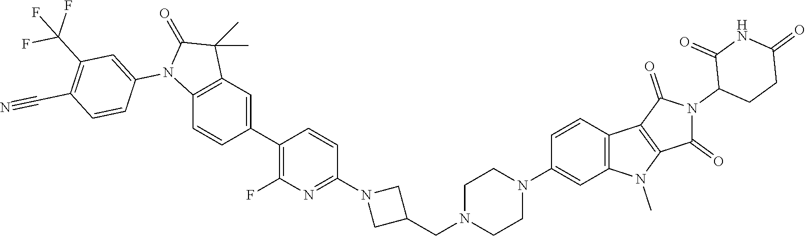 [SMILES: Cn1c2c(c3ccc(N4CCN(CC5CN(c6ccc(-c7ccc8c(c7)C(C)(C)C(=O)N8c7ccc(C#N)c(C(F)(F)F)c7)c(F)n6)C5)CC4)cc31)C(=O)N(C1CCC(=O)NC1=O)C2=O] |
| 7 | 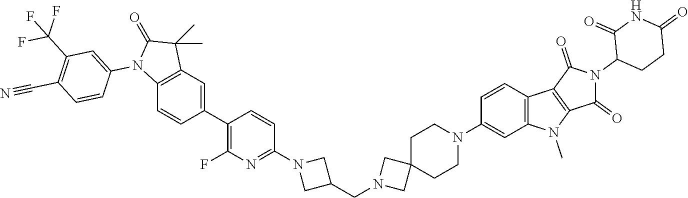 [SMILES: Cn1c2c(c3ccc(N4CCC5(CC4)CN(CC4CN(c6ccc(-c7ccc8c(c7)C(C)(C)C(=O)N8c7ccc(C#N)c(C(F)(F)F)c7)c(F)n6)C4)C5)cc31)C(=O)N(C1CCC(=O)NC1=O)C2=O] |
| 8 | 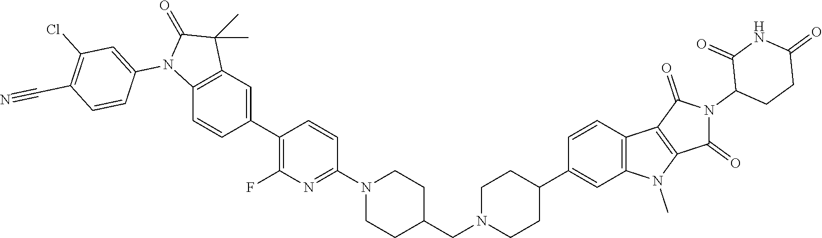 [SMILES: Cn1c2c(c3ccc(C4CCN(CC5CCN(c6ccc(-c7ccc8c(c7)C(C)(C)C(=O)N8c7ccc(C#N)c(Cl)c7)c(F)n6)CC5)CC4)cc31)C(=O)N(C1CCC(=O)NC1=O)C2=O] |
| 9 | 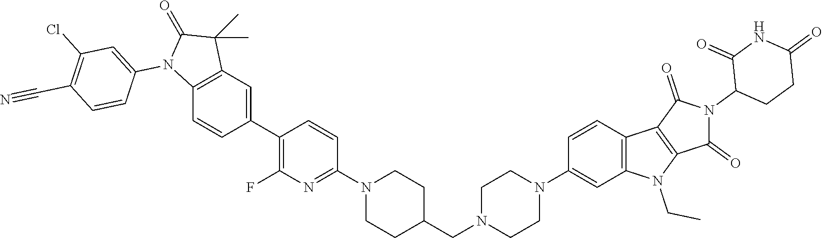 [SMILES: CCn1c2c(c3ccc(N4CCN(CC5CCN(c6ccc(-c7ccc8c(c7)C(C)(C)C(=O)N8c7ccc(C#N)c(Cl)c7)c(F)n6)CC5)CC4)cc31)C(=O)N(C1CCC(=O)NC1=O)C2=O] |
| 10 | 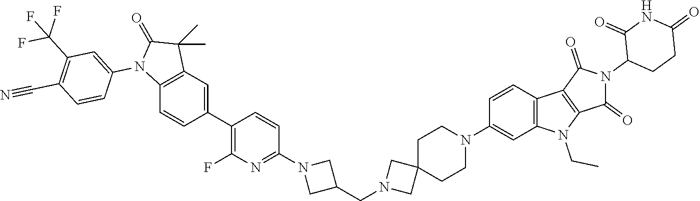 [SMILES: CCn1c2c(c3ccc(N4CCC5(CC4)CN(CC4CN(c6ccc(-c7ccc8c(c7)C(C)(C)C(=O)N8c7ccc(C#N)c(C(F)(F)F)c7)c(F)n6)C4)C5)cc31)C(=O)N(C1CCC(=O)NC1=O)C2=O] |
| 11 | 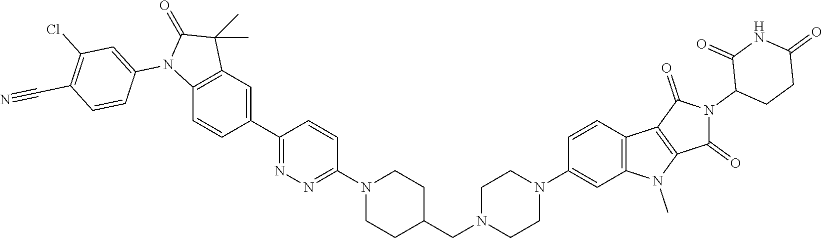 [SMILES: Cn1c2c(c3ccc(N4CCN(CC5CCN(c6ccc(-c7ccc8c(c7)C(C)(C)C(=O)N8c7ccc(C#N)c(Cl)c7)nn6)CC5)CC4)cc31)C(=O)N(C1CCC(=O)NC1=O)C2=O] |
| 12 | 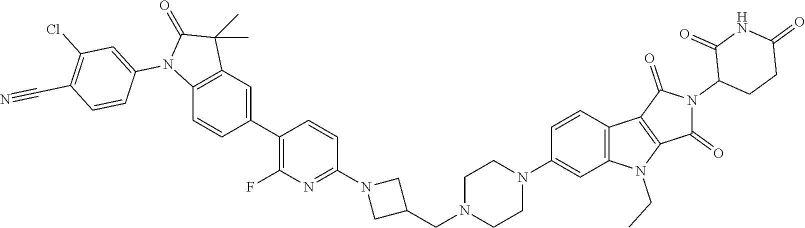 [SMILES: CCn1c2c(c3ccc(N4CCN(CC5CN(c6ccc(-c7ccc8c(c7)C(C)(C)C(=O)N8c7ccc(C#N)c(Cl)c7)c(F)n6)C5)CC4)cc31)C(=O)N(C1CCC(=O)NC1=O)C2=O] |
| 13 | 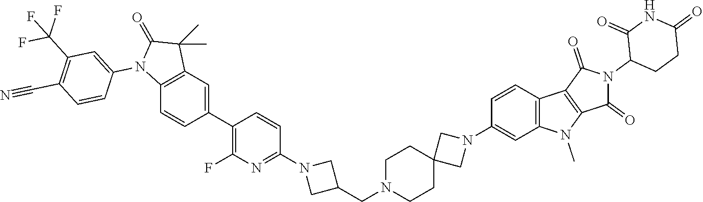 [SMILES: Cn1c2c(c3ccc(N4CC5(CCN(CC6CN(c7ccc(-c8ccc9c(c8)C(C)(C)C(=O)N9c8ccc(C#N)c(C(F)(F)F)c8)c(F)n7)C6)CC5)C4)cc31)C(=O)N(C1CCC(=O)NC1=O)C2=O] |
| 14 | 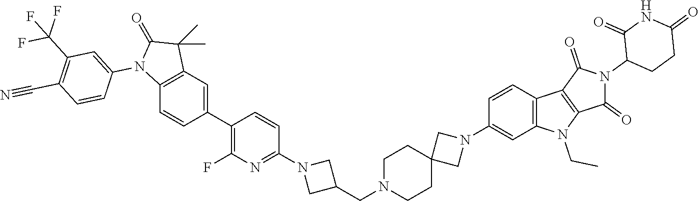 [SMILES: CCn1c2c(c3ccc(N4CC5(CCN(CC6CN(c7ccc(-c8ccc9c(c8)C(C)(C)C(=O)N9c8ccc(C#N)c(C(F)(F)F)c8)c(F)n7)C6)CC5)C4)cc31)C(=O)N(C1CCC(=O)NC1=O)C2=O] |
| 15 | 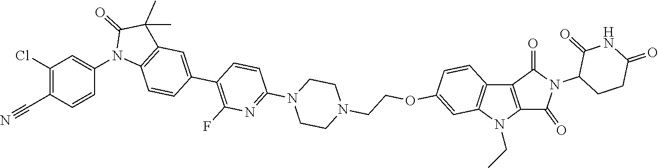 [SMILES: CCn1c2c(c3ccc(OCCN4CCN(c5ccc(-c6ccc7c(c6)C(C)(C)C(=O)N7c6ccc(C#N)c(Cl)c6)c(F)n5)CC4)cc31)C(=O)N(C1CCC(=O)NC1=O)C2=O] |
| 16 | 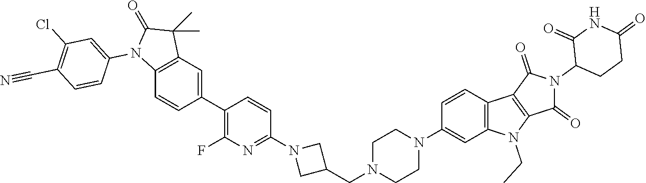 [SMILES: CCn1c2c(c3ccc(N4CCN(CC5CN(c6ccc(-c7ccc8c(c7)C(C)(C)C(=O)N8c7ccc(C#N)c(Cl)c7)c(F)n6)C5)CC4)cc31)C(=O)N(C1CCC(=O)NC1=O)C2=O] |

### Example 1—Synthesis of Bivalent Compounds

### Synthetic Method A—Synthesis of Compound 1:

### (1) Preparation of Intermediate I-A

The intermediate I-A was first prepared from commercially available 3-benzyloxy-phenylamine via the route shown below:

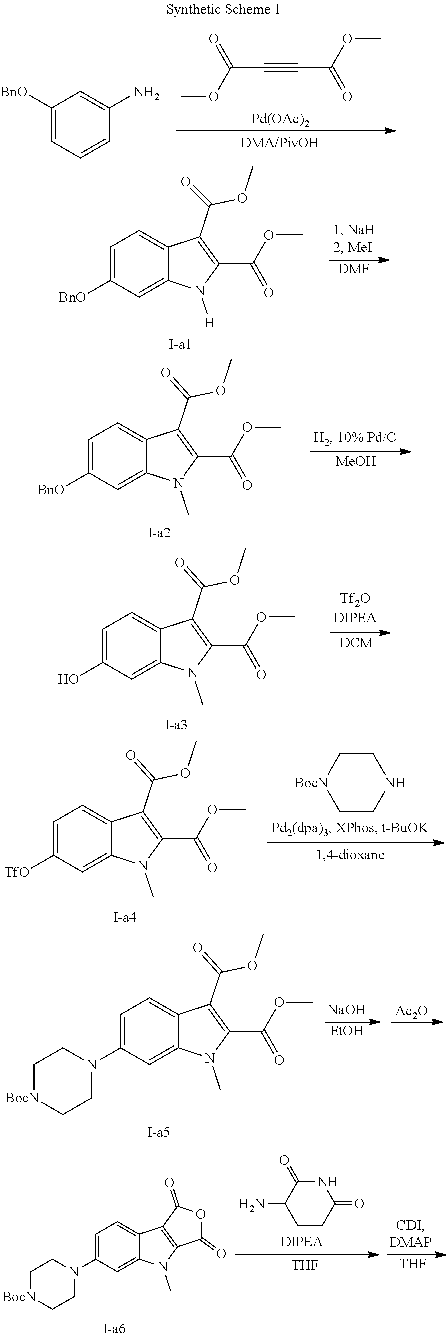

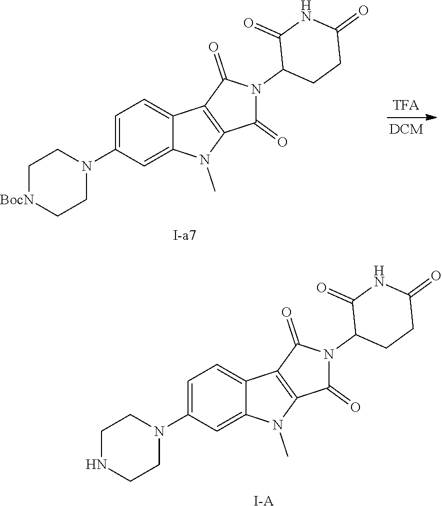 [SMILES: Cn1c2c(c3ccc(N4CCN(C(=O)OC(C)(C)C)CC4)cc31)C(=O)N(C1CCC(=O)NC1=O)C2=O ; Cn1c2c(c3ccc(N4CCNCC4)cc31)C(=O)N(C1CCC(=O)NC1=O)C2=O ; "I-A"]

Pd(OAc)₂ (262 mg, 20 mol %) was added to a mixture of I-a2 (1.7 g, 6.99 mmol), dimethyl acetylenedicarboxylate (0.71 mL, 5.8 mmol), and dimethylacetamide (DMA)/pivalic acid (PivOH) (4:1 v/v; 28 mL) in a 100 mL two-necks bottle, and the bottle then purged with air. The reaction mixture was gradually heated from room temperature to 120° C. for 8 h. After reaction was completed, the solution was then cooled to room temperature, diluted with ethyl acetate (30 mL), washed with H₂O (3×10 mL), dried over MgSO₄, filtered, and evaporated under vacuum to give a crude product. The crude product was purified by column chromatography on silica gel with an eluent (hexane:ethyl acetate=5:1) to afford a brown solid of 6-Benzyloxy-1H-indole-2,3-dicarboxylic acid dimethyl ester intermediate I-a1 (1.14 g, 50%).

An ice cold solution of 6-Benzyloxy-1H-indole-2,3-dicarboxylic acid dimethyl ester I-a1 (3.0 g, 8.84 mmol) in N,N-dimethylformamide (DMF) (15 ml) was treated with 60% NaH (0.53 g, 13.3 mmol) and methyl iodide (0.82 ml, 13.3 mmol) and then warmed to room temperature. After 1 h, the reaction mixture was partitioned between dichloromethane (DCM) (60 mL) and saturated aqueous NH₄Cl (20 mL). The combined organic layers were collected and washed with water (20 mL×3) and brine, then dried (with MgSO₄), filtered and concentrated to give a crude product. The crude product was purified by column chromatography on silica gel with an eluent (hexane:ethyl acetate=3:1) to afford a brown solid intermediate I-a2 (2.7 g, yield: 87%).

To a solution of 6-benzyloxy-1-methyl-1H-indole-2,3-dicarboxylic acid dimethyl ester I-a2 (5.0 g) in MeOH (150 mL), Pd/C (0.5 g) was added and stirred under hydrogen atmosphere for 2 h. After the reaction was completed, the mixture was passed through a celite pad and rinsed with Ethyl acetate, and the organic solvent was collected and removed on a rotor to afford an intermediate I-a3 (3.72 g, 100%).

To a stirred solution of 6-hydroxy-1-methyl-1H-indole-2,3-dicarboxylic acid dimethyl ester I-a3 (2.85 g, 10.83 mmol, 1.0 equiv.) in DCM (60 mL) was added N,N-diisopropylethylamine (DIPEA) (5.6 mL, 32.49 mmol, 3 equiv.) at 0° C. and the mixture was stirred at the same temperature for 10 minutes. Trifluoromethanesulfonic anhydride (Tf2O) (2.83 mL, 16.25 mmol, 1.5 equiv.) was added to the reaction dropwise and the resulting mixture was stirred at 0° C. for 1 hours. The reaction was diluted with DCM (40 mL) and washed with saturated NaHCO₃ (10 mL), saturated NH₄Cl (10 mL), water and brine. The organic layer was dried over MgSO₄, filtered, and concentrated to give a dark brown oil. The crude material was purified by column chromatography on silica gel eluting with 10% ethyl acetate in hexane to give the intermediate I-a4 (3.68 g, 86%)

To a stirred solution of 1-methyl-6-trifluoromethanesulfonyloxy-1H-indole-2,3-dicarboxylic acid dimethyl ester I-a4 (0.77 g, 1.94 mmol, 1.0 equiv.) in toluene (20 mL) was added tert-butyl piperazine-1-carboxylate (1.08 g, 5.82 mmol, 3.0 equiv.) XPhos (0.18 g, 20 mol %) and Cs₂CO₃ (1.9 g, 5.82 mmol, 3 eq.). The mixture was degassed with N₂ for 10 minutes and Pd(OAc)₂ (0.087 g, 20 mol %) was added to mixture. The resulting mixture was stirred at 80° C. for 17 h until no starting material left. The reaction was cooled to room temperature, diluted with ethyl acetate (30 mL) and water (10 mL), and the layers were separated. The aqueous layer was extracted with ethyl acetate (30 mL) and the combined organic layers were washed with brine, dried over MgSO₄, filtered, and concentrated. The crude material was purified by column chromatography on silica gel eluting with 25% ethyl acetate in hexane to give the intermediate I-a5 (0.72 g, 86%)

The I-a5 (0.65 g, 1.51 mmol) was treated with a solution of NaOH (0.6 g, 15.1 mmol) in EtOH (30 mL) and heated to reflux. After 4 h, the reaction mixture was cooled to ambient temperature, removed excess EtOH, diluted with ethyl acetate (30 mL) and acidified with 1 M HCl to pH about 3. The aqueous layer was then extracted with ethyl acetate (30 mL) and the combined organic layers were washed with brine, dried (with MgSO₄), filtered and concentrated to afford a white solid. Then the white solid and Ac₂O (5 mL) were combined and heated to 140° C. After 2 h, a mono-ester was found by LC-MASS, and the reaction mixture was cooled to ambient temperature. Then excess Ac₂O was removed, and then dried toluene was added and concentrated. The intermediate I-a6 was thus produced and then used without any further purification.

A solution of 3-Aminopiperidine-2,6-dione hydrochloride (0.11 g, 0.67 mmol, 2 eq) and DIPEA (0.23 mL, 1.34 mmol, 4 eq) in THF (2 mL) was stirred at room temperature for 30 min, then a solution of the intermediate I-a6 (0.13 g, 1 eq) in THF (2 mL) was added. The reaction was stirred at room temperature for 50 min, then the mixture was diluted with ethyl acetate (10 mL) and water (10 mL). 1N HCl_{(aq)} was then added until the pH value of aqueous layer was less than 2 (<2). The aqueous layer was extracted with ethyl acetate (10 mL×2), and the organic layers were combined, washed with brine, and dried with MgSO₄. The solvent was removed to give a crude material, and the crude material was used in the next step without purification. The above crude material was dissolved in THF (4 mL), and carbonyldiimidazole (CDI) (0.11 g, 0.67 mmol, 2 eq) and 4-dimethylaminopyridine (DMAP) (4 mg, 0.1 eq) were added. The reaction mixture was stirred at 50° C. for 2 h. After cooling down, the reaction mixture was diluted with ethyl acetate (10 mL) and water (10 mL). 1N HCl_{(aq)} was then added until the pH value of aqueous layer was less than 2 (<2), and the aqueous layer was then extracted with ethyl acetate (10 mL×2). The organic layers were collected, washed with brine, and dried over Na₂SO₄. The solvent was removed to give a crude product, and the crude product was purified by flash column chromatography with an eluent (Hexane/Ethyl acetate=1/1 to 1/2) to afford an intermediate I-a7 (0.28 g, 85%). Then the I-a7 and trifluoroacetic acid (TFA) (2.5 ml) were combined and stirred in CH₂Cl₂ (10 mL) at 0° C., then cooled to room temperature for 4 h. After reaction completed, the excess TFA was removed under vacuum to give a residue. Then the residue was diluted with DCM basified with Na₂CO₃ to pH 10 and the organics were washed with brine, dried (with Na₂SO₄), filtered and concentrated to afford an intermediate I-A, which was directly used without any purification.

### (2) Preparation of Intermediate I-B

The intermediate I-B was prepared from 2,6-Difluoro-pyridine via the route shown below:

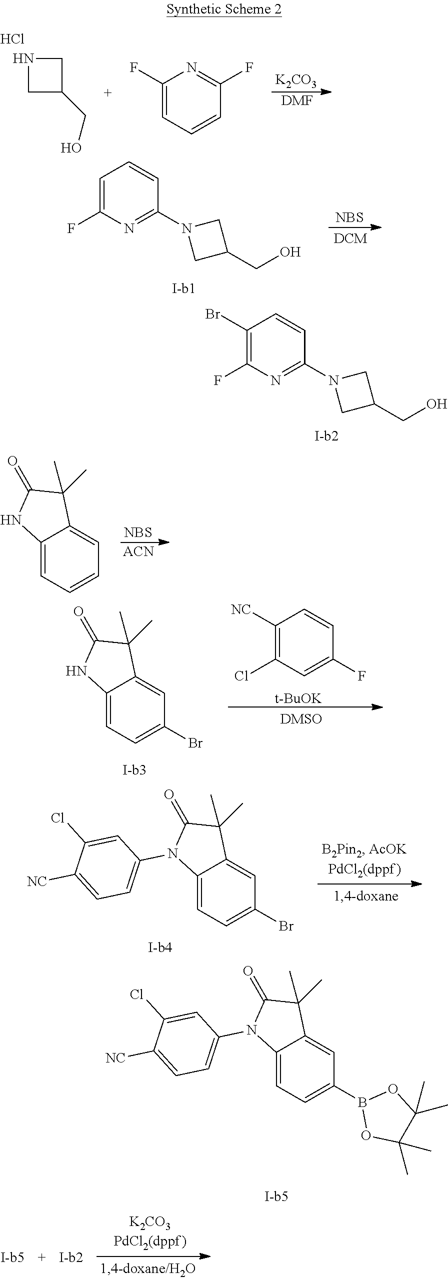

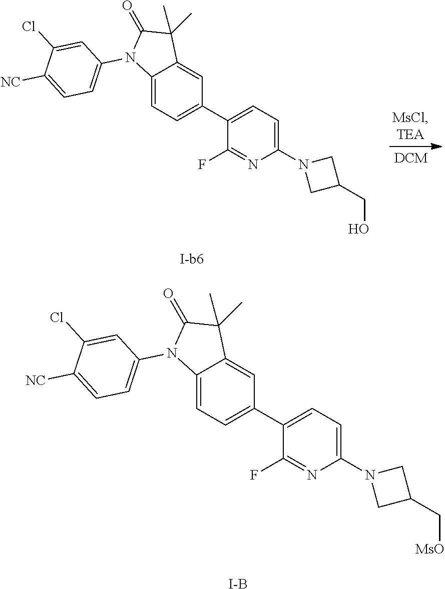 [SMILES: CC1(C)C(=O)N(c2ccc(C#N)c(Cl)c2)c2ccc(-c3ccc(N4CC(CO)C4)nc3F)cc21 ; CC1(C)C(=O)N(c2ccc(C#N)c(Cl)c2)c2ccc(-c3ccc(N4CC(COS(C)(=O)=O)C4)nc3F)cc21 ; "I-B"]

A solution of Azetidin-3-ylmethanol hydrochloride (1.0 g, 8.09 mmol) in DMF (6 mL) was sequentially added potassium carbonate (1.6 g, 11.58 mmol) and 2,6-difluoropyridine (0.5 g, 4.50 mmol) at room temperature. The reaction solution was stirred and reacted at 85° C. for 14 h. The reaction solution was added to water and extracted with ethyl acetate for three times. The organic phases were combined, washed with brine, dried over MgSO₄, and concentrated under reduced pressure to obtain a residue. The residue was purified by silica gel chromatography to obtain intermediate I-b1 (0.8 g, 98%).

A solution of I-b1 (319 mg, 1.75 mmol) in DCM (5 mL) was added N-bromosuccinimide (NBS) (280 mg, 1.57 mmol) under ice-bath and stirred for 10 min. After reaction completed, water was added to the reaction and the solution was extracted with DCM for three times. The organic phases were combined, washed with brine, dried over MgSO₄, filtered and concentrated under reduced pressure to obtain a residue. The residue was purified by silica gel chromatography to obtain intermediate I-b2 (410 mg, 74%).

1,3-Dihydro-3,3-dimethyl-2H-indol-2-one (1.5 g, 9.31 mmol) was dissolved in acetonitrile (35 mL) and cooled to 0° C. by ice bath. The resulting solution was added a solution of N-bromosuccinimide (NBS) (1.6 g, 9.31 mmol) in acetonitrile (ACN) (15 mL) dropwise. The mixture was allowed to reach room temperature and stirred for 4 h. The solvent was evaporated under reduced pressure and the residue was dissolved in dichloromethane (37 mL) and then washed with 1 N NaOH_{(aq)} (15 mL). The organic phase was separated, dried over MgSO₄, filtered and concentrated to obtain a residue. The residue was purified by silica gel chromatography to obtain intermediate I-b3 (1.8 g, 81%).

A solution of I-b3 (700 mg, 2.92 mmol) in DMSO was added potassium tert-butoxide (490 mg, 4.38 mmol) at room temperature. After the reaction mixture was stirred at room temperature for 30 min, 2-chloro-4-fluorobenzonitrile (544 mg, 3.50 mmol) was added to the mixture, and the reaction was stirred for 15 h at room temperature. The resulting solution was added water and ethyl acetate, the organic phase was separated, and the aqueous phase was further extracted with ethyl acetate for two times. The organic phase was combined and washed with brine, dried over MgSO₄, filtered, and concentrated. The residue was purified by silica gel chromatography to obtain intermediate I-b4 (670 mg, 61%).

The I-b4 (100 mg, 0.27 mmol) was dissolved in 1,4-dioxane (5 mL) and the solution was added bis(pinacolato)diboron (B₂Pin₂) (101 mg, 0.40 mmol), potassium acetate (78 mg, 0.80 mmol), and 1,1′-bis(diphenylphosphino)ferrocenedichloropalladium (II) (PdCl2(dppf) (20 mg, 0.03 mmol). The reaction mixture was stirred at 90° C. for 2 h under the protection of nitrogen. After the reaction completed, the solvent was evaporated under reduced pressure and the resulting residue was purified by silica gel chromatography to obtain intermediate I-b5 (110 mg, 96%).

A solution of I-b5 (200 mg, 0.47 mmol) in 1,4-dioxane (4 mL) and water (1 ml) was added I-b2 (112 mg, 0.43 mmol), potassium carbonate (178 mg, 1.29 mmol), and 1,1′-bis(diphenylphosphino)ferrocenedichloropalladium (II) (PdCl2(dppf) (29 mg, 0.04 mmol). The reaction mixture was stirred at 80° C. for 17 h under the protection of nitrogen. After the reaction completed, the 1,4-dioxane was evaporated under reduced pressure and the resulting residue was extracted with ethyl acetate for three times. The combined organic phase was dried over MgSO₄, filtered and concentrated to obtain a residue. The residue was then purified by silica gel chromatography to obtain intermediate I-b6 (205 mg, 92%).

To a solution of I-b6 (0.1 g, 0.21 mmol) in DCM (5 mL) was added triethylamine (TEA) (0.088 mL, 0.63 mmol) at room temperature. Then methane sulfonyl chloride (0.028 mL, 0.36 mmol) was added dropwise. The mixture reaction was stirred at room temperature for 1 h. After the reaction completed, diluted with DCM and extracted with NaHCO₃(aq) and NH₄Cl_{(aq)}. The organic phase was washed with bine, dried over Na₂SO₄₍ₛ₎, concentrated, and purified by silica gel chromatography to obtain intermediate I-B (115 mg, 86%).

(3) Preparation of Compound 1 from Intermediate I-A and Intermediate I-B

Compound 1 was prepared via the route shown below:

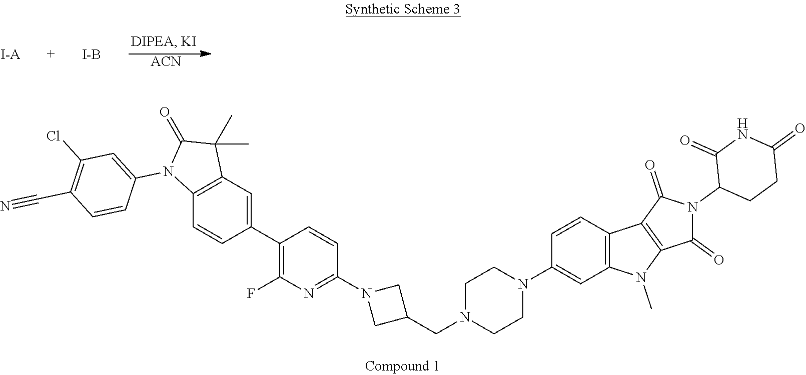 [SMILES: "I-A" ; "+" ; "I-B" ; "ACN" ; Cn1c2c(c3ccc(N4CCN(CC5CN(c6ccc(-c7ccc8c(c7)C(C)(C)C(=O)N8c7ccc(C#N)c(Cl)c7)c(F)n6)C5)CC4)cc31)C(=O)N(C1CCC(=O)NC1=O)C2=O]

To a solution of I-A (0.079 g, 0.20 mmol), I-B (0.11 g, 0.20 mmol), KI (0.10 g, 0.6 mmol, 3.0 eq) and DIPEA (0.17 mL, 1.0 mmol, 5.0 eq) in dry CH₃CN (2 mL) was heated to 75° C. for 17 h. After the reaction completed, diluted with ethyl acetate (10 mL) and basified with saturated Na₂CO₃ to pH about 10. The organic layer was washed with brine, dried over Na₂SO₄(s), concentrated and purified by column chromatography on silica gel to get Compound 1 (0.095 g, 58%). MS: m/z 854.6 (M⁺+1); ¹H NMR (DMSO-d6) δ 11.05 (s, 1H), 8.19 (d, 1H), 7.99 (d, 1H), 7.81 (dd, 1H), 7.74 (dd, 1H), 7.62 (s, 1H), 7.56 (d, 1H), 7.38 (d, 1H), 7.17 (d, 1H), 7.07 (s, 1H), 7.03 (d, 1H), 6.39 (d, 1H), 4.97 (dd, 1H), 4.14-4.11 (m, 2H), 3.93 (s, 3H), 3.70-3.68 (m, 2H), 3.29 (bs, 4H), 3.06-2.99 (m, 1H), 2.92-2.86 (m, 1H), 2.70-2.68 (m, 2H), 2.62-2.54 (m, 6H), 2.05-2.00 (m, 1H), 1.46 (s, 6H).

### Synthetic Method B—Synthesis of Compound 2:

### (1) Preparation of Intermediate I-C

The Intermediate I-C was first prepared from commercially available 4-Hydroxy-piperidine-1-carboxylic acid tert-butyl ester via the route shown below:

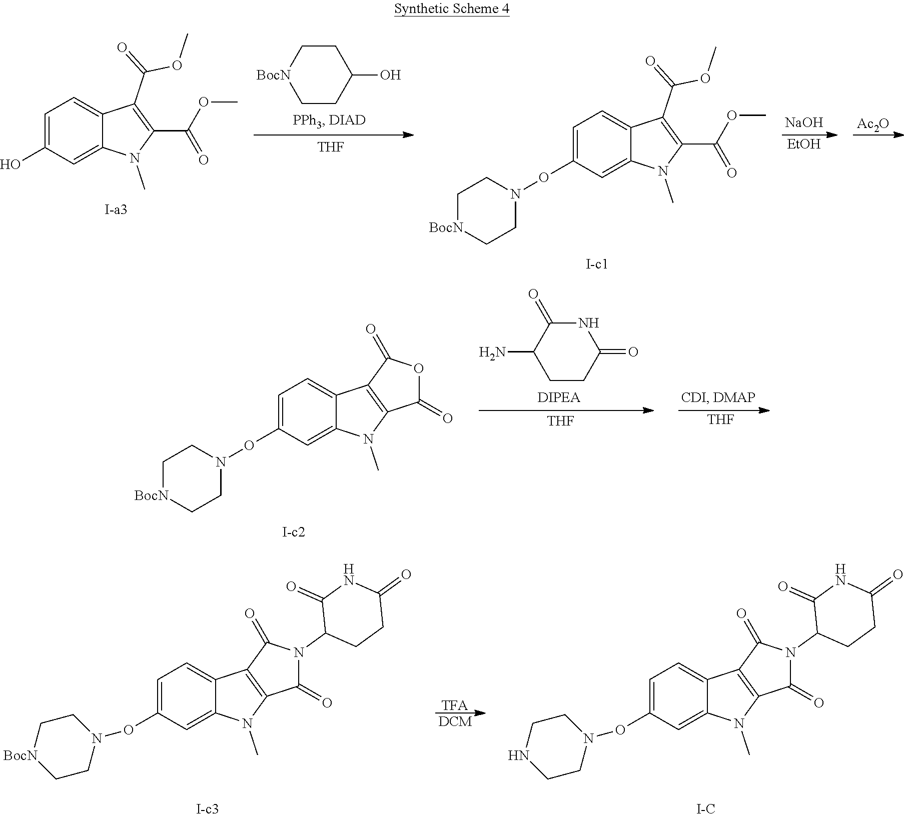

To a solution of 4-Hydroxy-piperidine-1-carboxylic acid tert-butyl ester (459 mg, 2.28 mmol), triphenylphosphine (747 mg, 2.85 mmol), and I-a3 (500 mg, 1.90 mmol) in tetrahydrofuran (THF) (20 ml), diisopropyl azodicarboxylate (DIAD) (0.56 ml, 2.85 mmol) was added dropwise. After addition of the DIAD, the mixture was stirred under nitrogen for 18 hours (h) to give a crude mixture. After the solvent was evaporated, ether was added to re-suspend the crude mixture, and the triphenylphosphine oxide was filtered off. The solid was washed with ether (10 mL), and the filtrate was evaporated to give a yellow oil. The yellow oil was purified by column chromatography on silica gel to give an intermediate I-c1 (910 mg, 89%).

The I-c1 (800 mg, 1.79 mmol) was treated with a solution of NaOH (717 mg, 17.9 mmol) in EtOH (25 mL) and heated to reflux. After 4 h, the reaction mixture was cooled to ambient temperature, removed excess EtOH, diluted with ethyl acetate and acidified with 1 M HCl to pH 3. The aqueous layer was then extracted with ethyl acetate and the organics were washed with brine, dried (with MgSO₄), filtered and concentrated to afford a white solid. Then the white solid and Ac₂O (5 mL) were combined and heated to 140° C. After 2 h, a mono-ester was found by LC-MASS, and the reaction mixture was cooled to ambient temperature. Then excess Ac₂O was removed, and then dried toluene was added and concentrated. The intermediate I-c2 was thus produced and then used without any further purification.

A solution of 3-Aminopiperidine-2,6-dione hydrochloride (329 mg, 2.0 mmol, 2 eq) and DIPEA (0.70 mL, 4.0 mmol, 4 eq) in THF (6 mL) was stirred at room temperature for 30 min, then a solution of I-c2 (400 mg, 1.0 mmol, 1 eq) in THF (6 mL) was added. The reaction was stirred at room temperature for 50 min, then the mixture was diluted with Ethyl acetate and water. 1N HCl_{(aq)} was then added until the pH value of aqueous layer was less than 2 (<2). The aqueous layer was extracted with Ethyl acetate twice, and the organic layers were combined, washed with brine, and dried with MgSO₄. The solvent was removed to give a crude material, and the crude material was used in the next step without purification. The above crude material was dissolved in THF (6 mL), and CDI (324 mg, 2.0 mmol, 2 eq) and DMAP (12 mg, 0.1 eq) were added. The reaction mixture was stirred at 50° C. for 2 h. After cooling down, the reaction mixture was diluted with Ethyl acetate (20 mL) and water (20 mL). 1N HCl_{(aq)} was then added until the pH value of aqueous layer was less than 2 (<2), and the aqueous layer was then extracted with Ethyl acetate (20 mL×2). The organic layers were collected, washed with brine, and dried over Na₂SO₄. The solvent was removed to give a crude product, and the crude product was purified by flash column chromatography with an eluent (Hexane/Ethyl acetate=1/1 to 1/2) to afford an intermediate I-c3 (428 mg, 84%). Then the I-c3 and TFA (2.5 ml) were combined and stirred in CH₂Cl₂ (10 mL) at 0° C., then cooled to room temperature for 4 h. After reaction completed, the excess TFA was removed under vacuum to give a residue. Then the residue was diluted with DCM basified with saturated Na₂CO₃ to pH about 10 and the organic layer was washed with brine, dried (with Na₂SO₄), filtered and concentrated to afford an intermediate I-C, which was directly used without any purification.

(2) Preparation of Compound 2 from Intermediate I-B and Intermediate I-C

Compound 2 was prepared via the route shown below:

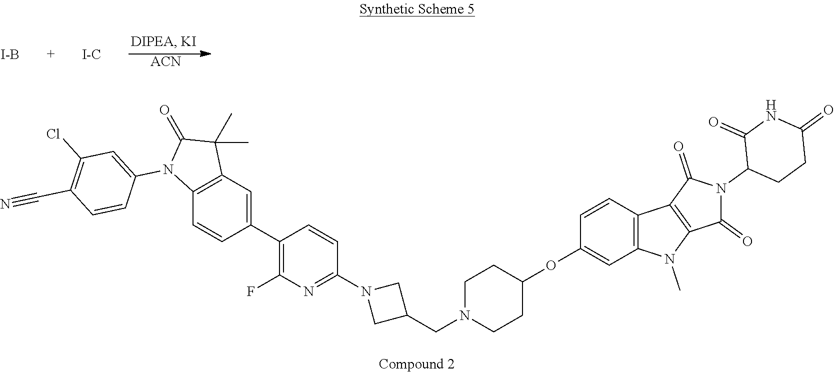 [SMILES: "I-B" ; "+" ; "I-C" ; "ACN" ; Cn1c2c(c3ccc(OC4CCN(CC5CN(c6ccc(-c7ccc8c(c7)C(C)(C)C(=O)N8c7ccc(C#N)c(Cl)c7)c(F)n6)C5)CC4)cc31)C(=O)N(C1CCC(=O)NC1=O)C2=O]

To a solution of I-B (133 mg, 0.24 mmol), I-C (98 mg, 0.24 mmol), KI (119 mg, 0.72 mmol, 3.0 eq) and DIPEA (0.21 mL, 1.2 mmol, 5.0 eq) in dry CH₃CN (ACN) (2 mL) was heated to 75° C. for 17 h. After the reaction completed, diluted with ethyl acetate (10 mL) and basified with saturated Na₂CO₃ to pH about 10. The organic layer was washed with brine, dried over Na₂SO₄(s), concentrated and purified by column chromatography on silica gel to get Compound 2 (134 mg, 64%). MS: m/z 869.3 (M⁺+1); ¹H NMR (DMSO-d₆) δ 11.06 (s, 1H), 8.19 (d, 1H), 7.99 (d, 1H), 7.81 (dd, 1H), 7.74 (dd, 1H), 7.62-7.61 (m, 2H), 7.38 (d, 1H), 7.31 (d, 1H), 7.04-7.01 (m, 2H), 6.37 (dd, 1H), 4.98 (dd, 1H), 4.59-4.54 (m, 1H), 4.10 (t, 2H), 3.96 (s, 3H), 3.67-3.65 (m, 2H), 2.99-2.94 (m, 1H), 2.92-2.86 (m, 1H), 2.78-2.71 (m, 2H), 2.67-2.62 (m, 2H), 2.62-2.54 (m, 1H), 2.54-2.49 (m, 1H), 2.36-2.27 (m, 2H), 2.06-2.00 (m, 3H), 1.71-1.66 (m, 2H), 1.45 (s, 6H).

### Synthetic Method C—Synthesis of Compound 3:

### (1) Preparation of Intermediate I-D

The intermediate I-D was first prepared from I-a1 via the route shown below:

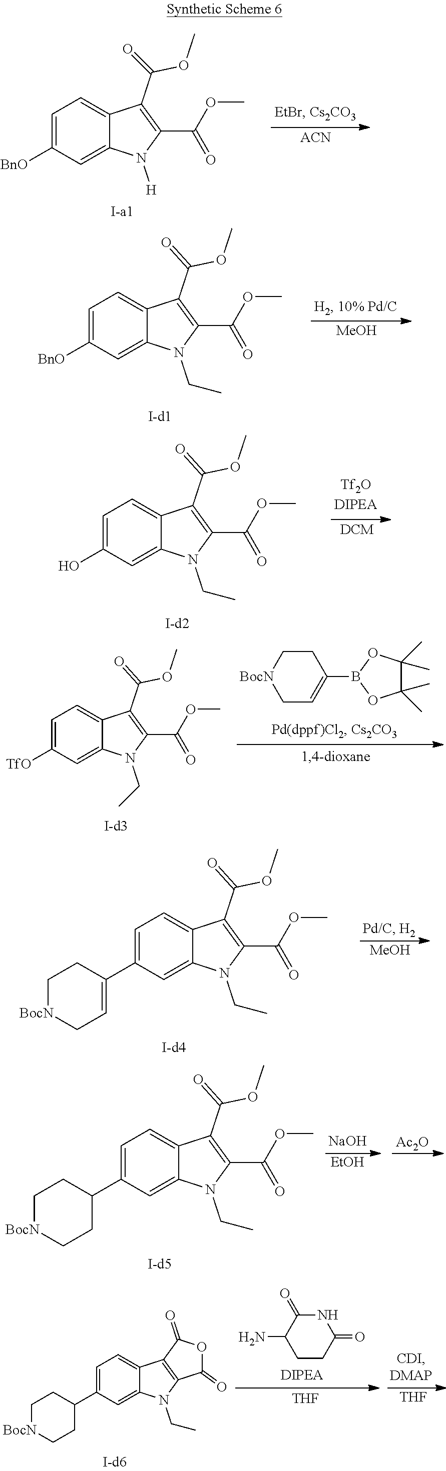

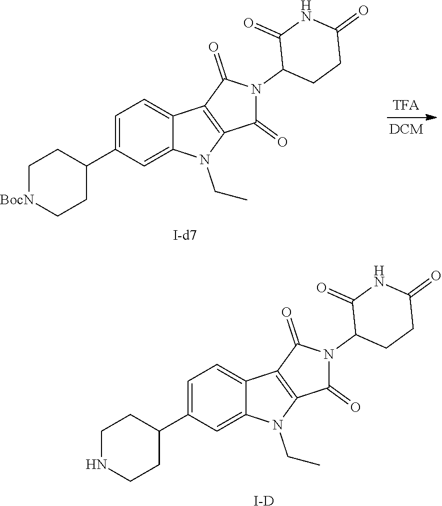 [SMILES: CCn1c2c(c3ccc(C4CCN(C(=O)OC(C)(C)C)CC4)cc31)C(=O)N(C1CCC(=O)NC1=O)C2=O ; CCn1c2c(c3ccc(C4CCNCC4)cc31)C(=O)N(C1CCC(=O)NC1=O)C2=O ; "I-D"]

To a solution of I-a1 (5.0 g, 14.73 mmol, 1 eq.) in ACN (50 mL) was added Cs₂CO₃ (9.6 g, 29.47 mmol, 2.0 eq) followed by bromoethane (1.65 mL, 22.10 mmol, 1.5 eq.) at 25° C. under N₂ atmosphere. The mixture was heated at 80° C. for 4 hours until no I-a1 left and cooled to room temperature. Solvent was reduced to half of the original volume, diluted with ethyl acetate (50 mL), and washed with H₂O (100 mL). Layers were separated and the aqueous layer was extracted with ethyl acetate (30 mL×2). The combined organic layers were washed with brine (100 mL), dried over MgSO₄, filtered, and concentrated. The crude material was purified by flash column chromatography on silica gel eluting with 15-20% EA/Hex to afford the I-d1 (4.8 g, 88%).

To a solution of I-d1 (1.0 g, 2.72 mmol) in 20 mL of MeOH was de-gas and added Pd/C (0.1 g, 10 wt %), then the reaction was bubbled with a balloon of hydrogen gas and stirred at rt for 2 h. While the reaction was completed, suspension reaction mixture was filtered through a plug of Celite, and the filter cake was washed with EA. The filtrate was concentrated by rotary evaporation to get I-d2 (0.75 g, 99%).

To a stirred solution of I-d2 (1.0 g, 3.61 mmol, 1.0 equiv.) in DCM (20 mL) was added DIPEA (1.88 mL, 10.82 mmol, 3 equiv.) at 0° C. and the mixture was stirred at the same temperature for 10 minutes. Tf₂O (0.91 mL, 5.4 mmol, 1.5 equiv.) was added to the reaction dropwise and the resulting mixture was stirred at 0° C. for 1 hours. The reaction was diluted with DCM (20 mL) and washed with sat'd NaHCO₃, sat'd NH₄C1, water and brine. The organic layer was dried over MgSO₄, filtered, and concentrated to give a dark brown oil. The crude material was purified by column chromatography on silica gel eluting with 10% ethyl acetate in hexane to give the title compound as pure product I-d3 (1.23 g, 83%)

To a stirred solution of I-d3 (704 mg, 1.72 mmol, 1.0 equiv.) in 1,4-dioxane (17 mL) was added 4-(4,4,5,5-Tetramethyl-[1,3,2]dioxaborolan-2-yl)-3,6-dihydro-2H-pyridine-1-carboxylic acid tert-butyl ester (800 mg, 2.58 mmol, 1.5 equiv.) and Cs₂CO₃ (1.06 g, 3.26 mmol, 1.9 eq.) at 25° C. The mixture was degassed with N₂ for 15 minutes and Pd(dppf)Cl₂ (63 mg, 0.09 mmol, 5 mol %) was added at 25° C. The reaction was stirred at 25° C. for 5 h until no starting material left. The reaction was diluted with ethyl acetate (30 mL) and H₂O (50 mL). Layers were separated and the aqueous layer was extracted with ethyl acetate (15 mL×2). The combined organic layers were washed with brine, dried over MgSO₄, filtered, and concentrated. The crude material was purified by column chromatography on silica gel eluting with 10-15% ethyl acetate in hexane to give the title compound as pure product I-d4 (605 mg, 80%, R_{f}=0.21 in EA/Hex=1/5).

To a solution of I-d4 (2.0 g) in MeOH (20 mL), Pd/C (0.2 g) was added and stirred under hydrogen atmosphere for 2 h. After the reaction was completed, the mixture was passed through a celite pad and rinsed with Ethyl acetate, and the organic solvent was collected and removed on a rotor to afford an intermediate I-d5 (2.0 g, 100%).

The I-d5 (0.67 g, 1.51 mmol) was treated with a solution of NaOH (0.6 g, 15.1 mmol) in EtOH (30 mL) and heated to reflux. After 4 h, the reaction mixture was cooled to ambient temperature, removed excess EtOH, diluted with Ethyl acetate (30 mL) and acidified with 1 M HCl to pH about 3. The aqueous layer was then extracted with Ethyl acetate (30 mL) and the organic layer was washed with brine, dried (with MgSO₄), filtered and concentrated to afford a solid. Then the solid and Ac₂O (5 mL) were combined and heated to 140° C. After 2 h, After the reaction was completed, the reaction mixture was cooled to ambient temperature. Then excess Ac₂O was removed, and then dried toluene was added and concentrated. The intermediate I-d6 was thus produced and then used without any further purification.

A solution of 3-Aminopiperidine-2,6-dione hydrochloride (0.11 g, 0.67 mmol, 2 eq) and DIPEA (0.23 mL, 4 eq) in THF (2 mL) was stirred at room temperature for 30 min, then a solution of the intermediate I-d6 (0.13 g, 0.33 mmol, 1 eq) in THF (2 mL) was added. The reaction was stirred at room temperature for 50 min, then the mixture was diluted with ethyl acetate (10 mL) and water (10 mL). 1N HCl_{(aq)} was then added until the pH value of aqueous layer was less than 2 (<2). The aqueous layer was extracted with ethyl acetate (10 mL×2), and the organic layers were combined, washed with brine, and dried with MgSO₄. The solvent was removed to give a crude material, and the crude material was used in the next step without purification. The above crude material was dissolved in THF (4 mL), and CDI (0.11 g, 2 eq) and DMAP (4 mg, 0.1 eq) were added. The reaction mixture was stirred at 50° C. for 2 h. After cooling down, the reaction mixture was diluted with ethyl acetate (10 mL) and water (10 mL). 1N HCl_{(aq)} was then added until the pH value of aqueous layer was less than 2 (<2), and the aqueous layer was then extracted with ethyl acetate (10 mL×2). The organic layers were collected, washed with brine, and dried over Na₂SO₄. The solvent was removed to give a crude product, and the crude product was purified by flash column chromatography with an eluent (Hexane/Ethyl acetate=1/1 to 1/2) to afford a compound I-d7 (0.13 g, 80%). Then the solid I-d7 and TFA (1.0 mL) were combined and stirred in CH₂Cl₂ (4.0 mL) at 0° C., then cooled to room temperature for 4 h. After reaction completed, the excess TFA was removed under vacuum to give a residue. Then the residue was diluted with DCM basified with NaHCO₃ to pH 10 and the organics were washed with brine, dried (with Na₂SO₄), filtered and concentrated to afford an intermediate I-D, which was directly used without any purification.

(2) Preparation of Compound 3 from Intermediate I-B and Intermediate I-D

Compound 3 was prepared via the route shown below:

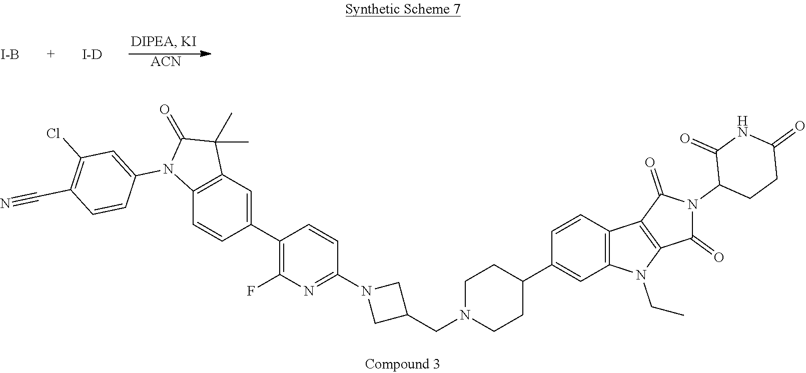 [SMILES: "I-B" ; "+" ; "I-D" ; "ACN" ; CCn1c2c(c3ccc(C4CCN(CC5CN(c6ccc(-c7ccc8c(c7)C(C)(C)C(=O)N8c7ccc(C#N)c(Cl)c7)c(F)n6)C5)CC4)cc31)C(=O)N(C1CCC(=O)NC1=O)C2=O]

To a solution of I-B (139 mg, 0.25 mmol), I-D (102 mg, 0.25 mmol), KI (124 mg, 0.75 mmol, 3.0 eq) and DIPEA (0.22 mL, 1.25 mmol, 5.0 eq) in dry CH₃CN (2 mL) was heated to 75° C. for 17 h. After the reaction completed, diluted with ethyl acetate (10 mL) and basified with saturated Na₂CO₃ to pH about 10. The organic layer was washed with brine, dried over Na₂SO₄(s), concentrated and purified by column chromatography on silica gel to get Compound 3 (141 mg, 65%). MS: m/z 867.1 (M⁺+1); ¹H NMR (DMSO-d₆) δ 11.07 (s, 1H), 8.19 (d, 1H), 7.99 (d, 1H), 7.81 (dd, 1H), 7.75 (dd, 1H), 7.72 (s, 1H), 7.67 (d, 1H), 7.62 (s, 1H), 7.38 (d, 1H), 7.30 (d, 1H), 7.03 (d, 1H), 6.39 (d, 1H), 5.00 (dd, 1H), 4.48 (q, 2H), 4.13-4.10 (m, 2H), 3.70-3.68 (m, 2H), 3.03-2.97 (m, 3H), 2.93-2.87 (m, 1H), 2.70-2.63 (m, 2H), 2.62-2.60 (m, 1H), 2.54-2.50 (m, 5H), 2.14-2.03 (m, 2H), 1.85-1.76 (m, 3H), 1.46 (s, 6H), 1.44 (t, 3H).

### Synthetic Method D—Synthesis of Compound 4:

(1) Preparation of Intermediate I-E

The intermediate I-E was first prepared from commercially available 3-Benzyloxy-propan-1-ol via the route shown below:

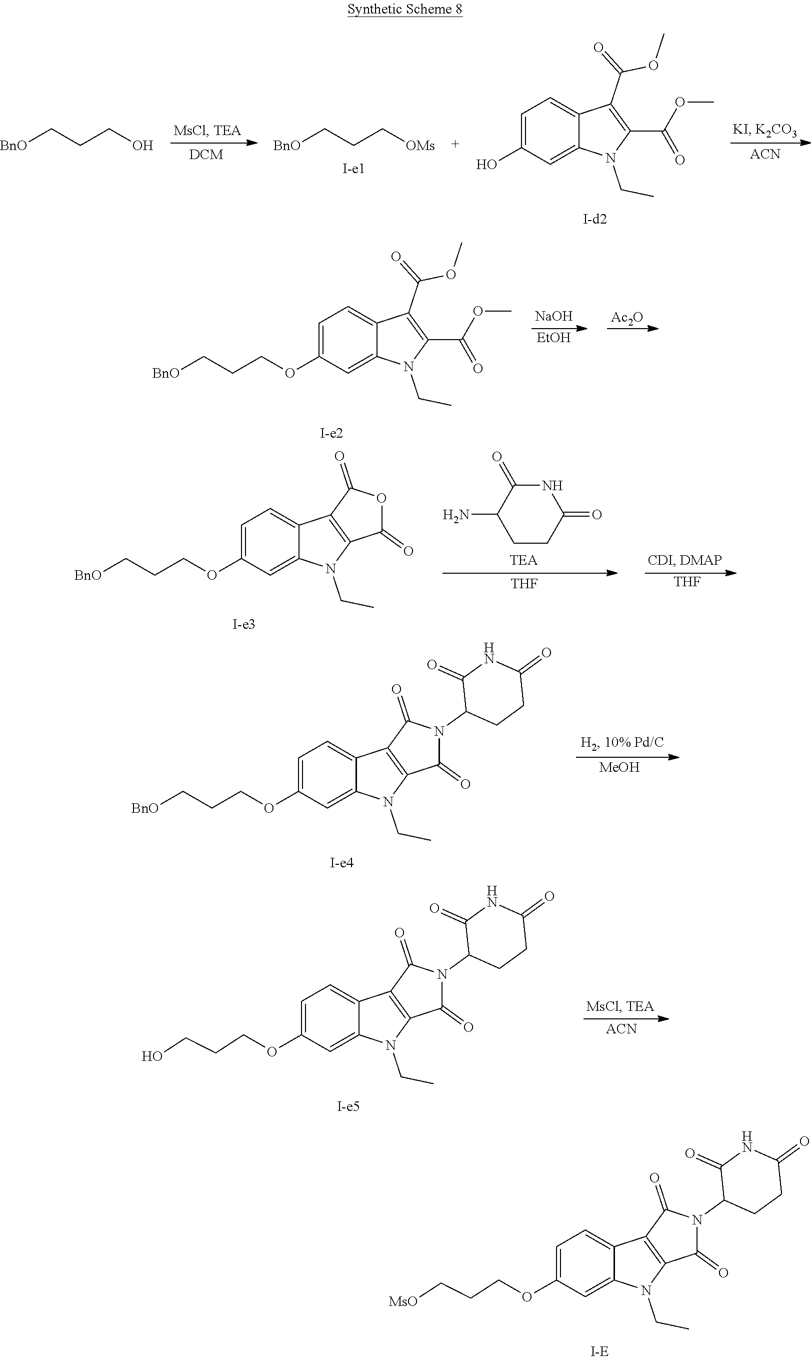

To a solution of 3-Benzyloxy-propan-1-ol (10.0 g, 60.2 mmol, 1 eq) in DCM (100 mL), triethylamine (TEA) (25.2 mL, 180.4 mmol, 3 eq) was added, and then methanesulfonyl chloride (MsCl) (7.0 mL, 90.3 mmol, 1.5 eq) was added under ice bath and stirred at room temperature for 1 h. The reaction mixture was diluted with DCM, washed with saturated NaHCO_{3(aq)} and saturated NH₄Cl_{(aq)}, then dried over MgSO₄. The solvent was removed to give a crude product, and the crude product was purified by flash column with an eluent (Hexane/Ethyl acetate=3/1 to 1/1) to afford a compound I-g1 (13.9 g, 94%). MS: m/z 267.3 (M⁺+23).

To a solution of I-d2 (0.34 g, 1.23 mmol) in dry ACN (4 mL) was added potassium carbonate (0.678 g, 4.90 mmol, 4 eq), then heated to 60° C. for 30 mins. Then I-e1 (0.36 g, 1.47 mmol, 1.2 eq) and KI (0.20 g, 1.23 mmol, 1.0 eq) was added at room temperature. The mixture was heated to 70° C. for 17 h. While the reaction was completed diluted with DCM, acidification by 1N HCl until PH-5, then extracted with DCM, brine and dried over MgSO₄(s), the organic layer was concentrated by rotary evaporation and purified by column chromatography by eluted Ethyl acetate/Hex=4/1 to get I-e2 (0.51 g, 91%).

To solution of I-e2 (0.51 g, 0.12 mmol) in EtOH (10 ml) was added NaOH(s) (0.45 g, 12.1 mmol, 10 eq), then the reaction was heated to 80° C. for 20 h. While the reaction was completed to remove EtOH by rotary evaporation. The result residue was diluted with water and acidification by 3N HCl until PH-2 under ice bath. The mixture was extracted with EA and brine, dried over Na₂SO₄(s), concentrated by rotary evaporation to get white solid. Then the white solid and Ac₂O (5 mL) were combined and heated to 140° C. After 2 h, a mono-ester was found by LC-MASS, and the reaction mixture was cooled to ambient temperature. Then excess Ac₂O was removed, and then dried toluene was added and concentrated. The intermediate I-e3 was thus produced and then used without any further purification.

To a solution of 3-Aminopiperidine-2,6-dione hydrochloride (0.32 g, 1.94 mmol, 2.0 eq) in dry THF (4.0 mL) was added triethylamine (TEA) (0.54 mL, 3.89 mmol, 4.0 eq) and stirred at room temperature for 0.5 h. Then a solution of I-e3 (0.37 g, 0.97 mmol) in dry THF (1.0 mL) was added at ambient temperature, and stirred at same temperature for 2 h. After reaction completed the reaction mixture was cooled to ambient temperature, diluted with Ethyl acetate (10 mL) and acidified with 3 M HCl to pH about 2. The organics were washed with brine, dried over Na₂SO₄. The solvent was removed to give a crude material, and the crude material was used in the next step without purification. The above crude material was dissolved in THF (7 mL), and CDI (0.31 g, 1.94 mmol, 2 eq) and DMAP (12 mg, 0.1 eq) were added. The reaction mixture was stirred at 50° C. for 2 h. After cooling down, the reaction mixture was diluted with Ethyl acetate (10 mL) and water (10 mL). 1N HCl_{(aq)} was then added until the pH value of aqueous layer was less than 2 (<2), and the aqueous layer was then extracted with Ethyl acetate (10 mL×2). The organic layers were collected, washed with brine, and dried over Na₂SO₄. The solvent was removed to give a crude product, and the crude product was purified by flash column chromatography with an eluent (Hexane/Ethyl acetate=1/1 to 1/2) to afford I-e4 (0.48 g, 96%).

To a solution of I-e4 (0.30 g, 0.61 mmol) in EA/MeOH=4/1 (60 mL) was added Pd/C (60 mg) and stirred under hydrogen atmosphere for 1 h. After the reaction was completed, the mixture was passed through a pad of celite (rinsed with EA), and the solvent was removed rotary evaporation to afford I-e5 (0.24 g, 99%).

To a solution of I-e5 (0.1 g, 0.25 mmol) in DCM (2 mL) was added TEA (0.139 mL, 1.0 mmol, 4.0 eq) at room temperature. Then methane sulfonyl chloride (0.0387 mL, 0.5 mmol, 2.0 eq) was added dropwise. The mixture reaction was stirred at room temperature for 1-2 h. After the reaction completed, diluted with DCM (10 mL) and washed with saturated NaHCO₃(aq) and saturated NH₄Cl_{(aq)}, The organic layer was washed with bine, dried over Na₂SO₄(s), concentrated, and purified by flash column chromatography with an eluent (DCM/Acetone=10/1), to afford I-E (0.1 g, 84%).

(2) Preparation of Intermediate I-F

The intermediate I-F was first prepared from commercially available 2,6-Difluoro-pyridine via the route shown below:

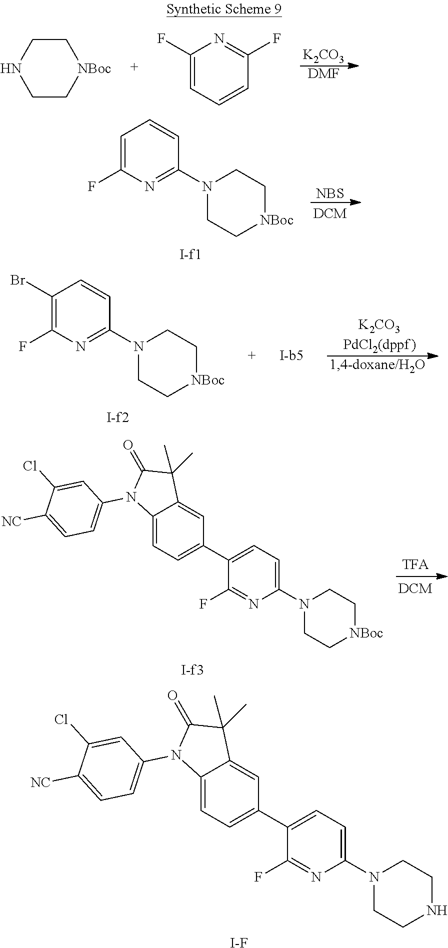

A solution of Piperazine-1-carboxylic acid tert-butyl ester (971 mg, 5.21 mmol) in DMF (10 mL) was sequentially added potassium carbonate (1.5 g, 10.86 mmol) and 2,6-difluoropyridine (500 mg, 4.35 mmol) at room temperature. The reaction solution was stirred and reacted at 85° C. for 14 h. The reaction solution was added to water and extracted with ethyl acetate for three times. The organic phases were combined, washed with brine, dried over MgSO₄, and concentrated under reduced pressure to obtain a residue. The residue was purified by silica gel chromatography to obtain I-f1 (890 mg, 73%).

A solution of I-f1 (319 mg, 1.75 mmol) in DCM (5 mL) was added N-bromosuccinimide (280 mg, 1.57 mmole) under ice-bath and stirred for 10 min. After reaction completed, water was added to the reaction and the solution was extracted with DCM for three times. The organic phases were combined, washed with brine, dried over MgSO₄, filtered and concentrated under reduced pressure to obtain a residue. The residue was purified by silica gel chromatography to obtain I-f2 (410 mg, 74%).

A solution of I-b5 (500 mg, 1.20 mmol) in 1,4-dioxane (3 mL) and water (0.75 ml) was added I-f2 (510 mg, 1.40 mmole), potassium carbonate (490 mg, 3.50 mmol), and 1,1′-bis(diphenylphosphino)ferrocenedichloropalladium(II) (87 mg, 0.1 mmol). The reaction mixture was stirred at 80° C. for 17 h under the protection of nitrogen. After the reaction completed, the 1,4-dioxane was evaporated under reduced pressure and the resulting residue was extracted with ethyl acetate (10 mL×3). The combined organic phase was dried over MgSO₄, filtered and concentrated to obtain a residue. The residue was then purified by silica gel chromatography to obtain I-f3 (530 mg, 77%). Then the solid I-f3 and CF₃COOH (2.5 mL) were combined and stirred in CH₂Cl₂ (10 mL) at 0° C., then cooled to room temperature for 4 h. After reaction completed, the excess CF₃COOH was removed under vacuum to give a residue. Then the residue was diluted with DCM basified with saturated Na₂CO₃(aq) to pH 10 and the organic layer was washed with brine, dried (with Na₂SO₄), filtered and concentrated to afford an intermediate I-F, which was directly used without any purification.

(3) Preparation of Compound 4 from Intermediate I-E and Intermediate I-F

Compound 4 was prepared via the route shown below:

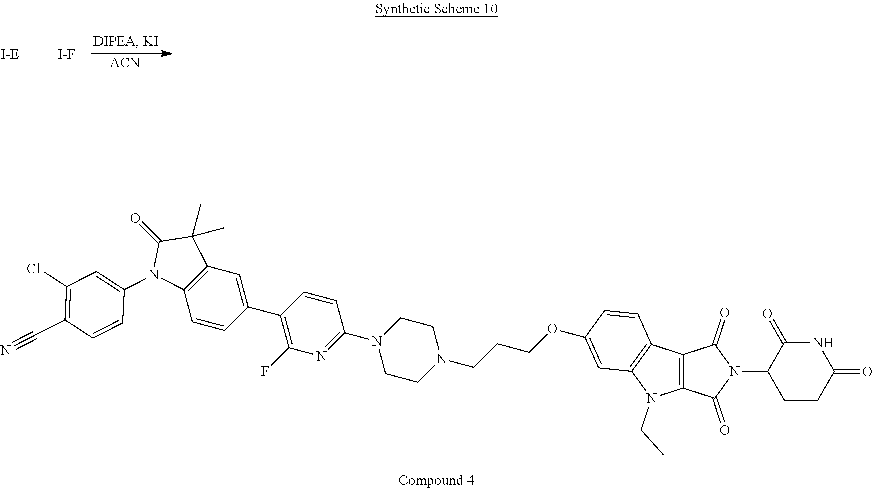 [SMILES: "I-F" ; "ACN" ; CCn1c2c(c3ccc(OCCCN4CCN(c5ccc(-c6ccc7c(c6)C(C)(C)C(=O)N7c6ccc(C#N)c(Cl)c6)c(F)n5)CC4)cc31)C(=O)N(C1CCC(=O)NC1=O)C2=O]

To a solution of I-E (100 mg, 0.21 mmol), I-F (100 mg, 0.21 mmol), KI (104 mg, 0.63 mmol, 3.0 eq) and DIPEA (0.18 mL, 1.05 mmol, 5.0 eq) in dry CH₃CN (2 mL) was heated to 75° C. for 17 h. After the reaction completed, diluted ethyl acetate (10 mL) and basified with saturated Na₂CO_{3(aq)} to pH about 10. The organic layer was washed with brine, dried over Na₂SO₄(s), concentrated and purified by column chromatography on silica gel get Compound 4 (145 mg, 80%). MS: m/z 857.7 (M⁺+1); ¹H NMR (DMSO-d₆) δ 11.06 (s, 1H), 8.19 (d, 1H), 7.99 (d, 1H), 7.85 (dd, 1H), 7.75 (dd, 1H), 7.64-7.62 (m, 2H), 7.41 (d, 1H), 7.35 (d, 1H), 7.04-7.01 (m, 2H), 6.83 (d, 1H), 4.98 (dd, 1H), 4.44 (q, 2H), 4.17 (t, 2H), 3.57-3.52 (m, 4H), 2.92-2.86 (m, 1H), 2.62-2.57 (m, 2H), 2.56-2.50 (m, 6H), 2.08-2.02 (m, 1H), 2.01-1.97 (m, 2H), 1.46 (s, 6H), 1.42 (t, 3H).

### Synthetic Method E—Synthesis of Chiral Intermediate I-G:

The intermediate I-G was prepared from I-a6 via the route shown below:

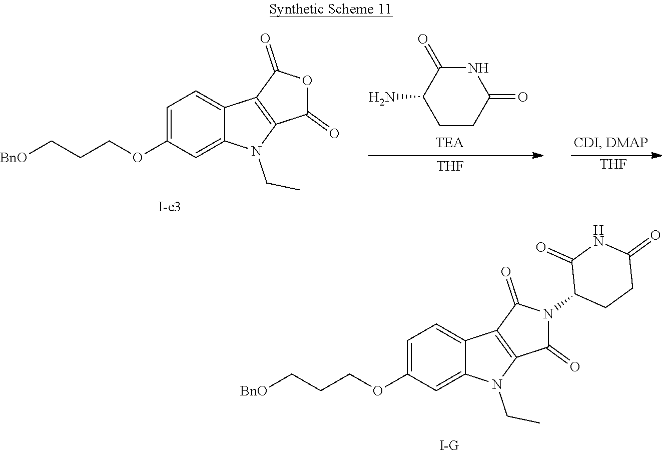 [SMILES: "THF" ; "THF" ; CCn1c2c(c3ccc(OCCCOCc4ccccc4)cc31)C(=O)OC2=O ; N[C@H]1CCC(=O)NC1=O ; CCn1c2c(c3ccc(OCCCOCc4ccccc4)cc31)C(=O)N([C@H]1CCC(=O)NC1=O)C2=O]

To a solution of (S) 3-Aminopiperidine-2,6-dione hydrochloride (0.32 g, 1.94 mmol, 2.0 eq) in dry THF (4.0 mL) was added triethylamine (TEA) (0.54 mL, 3.89 mmol, 4.0 eq) and stirred at room temperature for 0.5 h. Then a solution of I-e3 (0.37 g, 0.97 mmol) in dry THF (1.0 mL) was added at ambient temperature, and stirred at same temperature for 2 h. After reaction completed the reaction mixture was cooled to ambient temperature, diluted with Ethyl acetate (10 mL) and acidified with 3 M HCl to pH about 2. The organics were washed with brine, dried over Na₂SO₄. The solvent was removed to give a crude material, and the crude material was used in the next step without purification. The above crude material was dissolved in THF (7 mL), and CDI (0.31 g, 1.94 mmol, 2 eq) and DMAP (12 mg, 0.1 eq) were added. The reaction mixture was stirred at 50° C. for 2 h. After cooling down, the reaction mixture was diluted with Ethyl acetate (10 mL) and water (10 mL). 1N HCl_{(aq)} was then added until the pH value of aqueous layer was less than 2 (<2), and the aqueous layer was then extracted with Ethyl acetate (10 mL×2). The organic layers were collected, washed with brine, and dried over Na₂SO₄. The solvent was removed to give a crude product, and the crude product was purified by flash column chromatography with an eluent (Hexane/Ethyl acetate=1/1 to 1/2) to afford I-G (0.44 g, 93%).

The preparation of Compound 1, 2, 3 and 4 of the present invention is exemplified above. Other compounds in the present invention can be synthesized by similar methods shown in Synthetic Method A (Synthetic schemes 1 to 3), Synthetic Method B (Synthetic schemes 4 to 5), Synthetic Method C (Synthetic schemes 6 to 7), Synthetic Method D (Synthetic schemes 8 to 10), Synthetic Method E or by any known synthesis methods based on the general knowledge of organic chemistry.

Following Synthetic Method A (Synthetic schemes 1 to 3), Synthetic Method B (Synthetic schemes 4 to 5), Synthetic Method C (Synthetic schemes 6 to 7) or Synthetic Method D (Synthetic schemes 8 to 10) compound 5-16 were prepared by a method similar to Synthetic Method A, Synthetic Method B, Synthetic Method C, Synthetic Method D or Synthetic Method E with changing one or more starting materials to obtain the desired products.

Compound 5: MS: m/z 882.8 (M⁺+1); ¹H NMR (DMSO-d₆) δ 11.05 (s, 1H), 8.19 (d, 1H), 8.00 (d, 1H), 7.80 (dd, 1H), 7.74 (dd, 1H), 7.61 (s, 1H), 7.56 (d, 1H), 7.38 (d, 1H), 7.17 (d, 1H), 7.06 (s, 1H), 7.03 (d, 1H), 6.44 (d, 1H), 4.96 (dd, 1H), 3.93 (s, 3H), 3.69-3.65 (m, 1H), 3.56-3.53 (m, 1H), 3.38-3.35 (m, 1H), 3.29 (bs, 4H), 3.05-3.02 (m, 1H), 2.95-2.86 (m, 1H), 2.62-2.55 (m, 5H), 2.47-2.42 (m, 1H), 2.34-2.28 (m, 1H), 2.19-2.14 (m, 1H), 2.05-2.01 (m, 1H), 1.70-1.64 (m, 3H), 1.45 (s, 6H), 1.27-1.23 (m, 2H).

Compound 6: MS: m/z 901.8 (M⁺+1).

Compound 7: MS: m/z 927.8 (M⁺+1).

Compound 8: MS: m/z 881.5 (M⁺+1); ¹H NMR (DMSO-d₆) δ 11.07 (s, 1H), 8.19 (d, 1H), 7.99 (d, 1H), 7.81 (dd, 1H), 7.74 (dd, 1H), 7.69-7.62 (m, 3H), 7.39 (d, 1H), 7.31 (d, 1H), 7.03 (d, 1H), 6.45 (d, 1H), 5.00 (dd, 1H), 4.00 (s, 3H), 3.71-3.65 (m, 1H), 3.59-3.52 (m, 1H), 3.11-3.00 (m, 3H), 2.92-2.87 (m, 1H), 2.63-2.59 (m, 1H), 2.59-2.49 (m, 5H), 2.35-2.28 (m, 1H), 2.22-2.13 (m, 1H), 2.09-1.99 (m, 3H), 1.88-1.74 (m, 3H), 1.73-1.60 (m, 2H), 1.46 (s, 6H), 1.29-1.22 (m, 2H).

Compound 9: MS: m/z 896.8 (M⁺+1); ¹H NMR (DMSO-d₆) δ 11.05 (s, 1H), 8.19 (d, 1H), 7.99 (d, 1H), 7.80 (dd, 1H), 7.74 (dd, 1H), 7.61 (s, 1H), 7.56 (d, 1H), 7.38 (d, 1H), 7.16 (d, 1H), 7.12 (s, 1H), 7.03 (d, 1H), 6.44 (d, 1H), 4.96 (dd, 1H), 4.41 (q, 2H), 3.68-3.65 (m, 1H), 3.56-3.53 (m, 1H), 3.37-3.35 (m, 1H), 3.29 (bs, 4H), 3.05-3.02 (m, 1H), 2.92-2.86 (m, 1H), 2.62-2.55 (m, 5H), 2.47-2.42 (m, 1H), 2.34-2.28 (m, 1H), 2.19-2.14 (m, 1H), 2.07-2.01 (m, 1H), 1.70-1.64 (m, 3H), 1.45 (s, 6H), 1.41 (t, 3H), 1.27-1.23 (m, 2H).

Compound 10: MS: m/z 941.6 (M⁺+1).

Compound 11: MS: m/z 865.9 (M⁺+1); ¹H NMR (DMSO-d₆) δ 11.05 (s, 1H), 8.20 (d, 1H), 8.16 (s, 1H), 8.00 (d, 1H), 7.94-7.92 (m, 2H), 7.76 (dd, 1H), 7.56 (d, 1H), 7.17 (d, 1H), 7.08-7.07 (m, 2H), 6.95 (d, 1H), 4.96 (dd, 1H), 3.93 (s, 3H), 3.80-3.77 (m, 1H), 3.69-3.65 (m, 1H), 3.47-3.43 (m, 1H), 3.20-3.28 (m, 4H), 3.15-3.11 (m, 1H), 2.92-2.86 (m, 1H), 2.63-2.56 (m, 5H), 2.48-2.44 (m, 1H), 2.37-2.33 (m, 1H), 2.23-2.18 (m, 1H), 2.06-2.00 (m, 1H), 1.73-1.67 (m, 3H), 1.48 (s, 6H), 1.28-1.23 (m, 2H).

Compound 12: MS: m/z 868.4 (M⁺+1); ¹H NMR (DMSO-d₆) δ 11.05 (s, 1H), 8.19 (d, 1H), 7.99 (d, 1H), 7.81 (dd, 1H), 7.74 (dd, 1H), 7.62 (bs, 1H), 7.56 (d, 1H), 7.38 (d, 1H), 7.16 (d, 1H), 7.13 (s, 1H), 7.03 (d, 1H), 6.39 (d, 1H), 4.96 (dd, 1H), 4.13-4.11 (m, 2H), 3.70-3.68 (m, 2H), 3.29 (bs, 4H), 3.06-3.00 (m, 1H), 2.92-2.86 (m, 1H), 2.70-2.68 (m, 2H), 2.62-2.54 (m, 6H), 2.07-2.02 (m, 1H), 1.46 (s, 6H), 1.41 (t, 3H).

Compound 13: MS: m/z 927.6 (M⁺+1).

Compound 14: MS: m/z 941.8 (M⁺+1).

Compound 15: MS: m/z 843.7 (M⁺+1); ¹H NMR (DMSO-d₆) δ 11.06 (s, 1H), 8.19 (d, 1H), 7.99 (bs, 1H), 7.85 (dd, 1H), 7.75 (d, 1H), 7.64-7.62 (m, 2H), 7.42-7.40 (m, 2H), 7.05-7.02 (m, 2H), 6.84 (d, 1H), 4.98 (dd, 1H), 4.45 (q, 2H), 4.27 (t, 2H), 3.60-3.53 (m, 4H), 2.92-2.86 (m, 1H), 2.86-2.82 (m, 2H), 2.64 (bs, 4H), 2.62-2.56 (m, 2H), 2.08-2.02 (m, 1H), 1.46 (s, 6H), 1.44 (t, 3H).

Compound 16: MS: m/z 902.5 (M⁺+1); ¹H NMR (DMSO-d₆) δ 11.05 (s, 1H), 8.22 (s, 1H), 8.09 (dd, 1H), 7.84-7.80 (m, 1H), 7.63 (s, 1H), 7.56 (d, 1H), 7.39 (d, 1H), 7.16 (d, 1H), 7.13 (s, 1H), 7.08 (d, 1H), 6.39 (d, 1H), 4.96 (dd, 1H), 4.41 (q, 2H), 4.14-4.11 (m, 2H), 3.70-3.68 (m, 2H), 3.29 (bs, 4H), 3.06-2.99 (m, 1H), 2.92-2.86 (m, 1H), 2.70-2.68 (m, 2H), 2.62-2.57 (m, 5H), 2.55-2.50 (m, 1H), 2.07-2.02 (m, 1H), 1.47 (s, 6H), 1.41 (t, 3H).

### Example 2—Androgen Receptor Degradation Assay Using Western Blot Analysis

LNCaP.FGC cells (Cat. 60088, Bioresource Collection and Research Center, 15 HsinChu City, Taiwan R.O.C.) grown in RPMI 1640 (Cat. 31800022, Thermo Fisher Scientific, Waltham, Massachusetts, United States) medium supplemented with 10% FBS (Cat. 10437028, Thermo Fisher Scientific, Waltham, Massachusetts, United States), 10 mM HEPES (Cat. 15630080, Thermo Fisher Scientific, Waltham, Massachusetts, United States) and 1 mM sodium pyruvate (Cat. 11360070, Thermo 20 Fisher Scientific, Waltham, Massachusetts, United States) were seeded at 2×105 cells per well in 24-well tissue culture plates. Cells were incubated at 37° C., 5% CO2 for 24 hours (hr), then treated with 100 nanomolar concentrations (nM) any of the compounds 1 to 148 and ARV-110 for 24 hr. After the treatment, the cells were harvested, washed by PBS, and lysed with RIPA lysis and extraction buffer (Cat. 25 89900, Thermo Fisher Scientific, Waltham, Massachusetts, United States) supplemented with Halt Protease Inhibitor Cocktail (Cat. 78430, Thermo Fisher Scientific, Waltham, Massachusetts, United States) to collect protein samples. Cells not treated with any of the above-mentioned compounds are used as a negative control.

The protein samples were separated by polyacrylamide gel electrophoresis, and then transferred to a piece of Immuno-Blot PVDF membrane (Cat. 1620177, Bio-Rad Laboratories, Hercules, California, United States). The presence of androgen receptor in the protein samples was detected by standard Western blotting procedure using an anti-AR antibody (1:2000 dilution) (Cat. 5153, Cell Signaling Technology Inc., Danvers, Massachusetts, United States) and a goat anti-rabbit HRP-conjugated secondary antibody (1:5000 dilution) (C04003, Croyez Bioscience Co., Ltd., Taipei City, Taiwan R.O.C.). The internal loading control GAPDH was detected using a mouse monoclonal antibody (1:5000) (GTX627408, GeneTex International Corp., HsinChu City, Taiwan R.O.C.) and a goat anti-mouse HRP-conjugated secondary antibody (1:5000 dilution) (C04001, Croyez Bioscience Co., Ltd., Taipei City, Taiwan R.O.C.). Chemiluminescence signals were developed using Clarity Western ECL substrate (Cat. 1705061, Bio-Rad Laboratories, Hercules, California, United States) and detected with digital imager iBright FL1500 (Invitrogen Corp., Carlsbad, California, United States).

Compounds 1-16 were selected and serial diluted (10×) with RPMI medium for treating LNCaP.FGC cells. A calibration curve was determined by the serial diluted samples for each compound, and the concentration needed for 50% AR degradation (AR DC50) for each compound was calculated. The results are shown in Table 2. “Cpd. ID” indicates the compound ID number in Table 1.

TABLE 2
AR DC₅₀ (nM) of Compounds
of the Present Invention

| Cpd. ID. | AR DC₅₀ (nM) |
|---|---|
| 1 | B |
| 2 | C |
| 3 | B |
| 4 | B |
| 5 | C |
| 6 | — |
| 7 | — |
| 8 | B |
| 9 | B |
| 10 | |
| 11 | C |
| 12 | A |
| 13 | — |
| 14 | — |
| 15 | B |
| 16 | C |

*AR DC50 (nM) A < 200; 200 ≤ B < 1000; C ≥ 1000

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A bivalent compound comprising a target binding moiety (TBM) and a cereblon E3 ubiquitin ligase binding moiety (CLM) represented by Formula (II)-1: 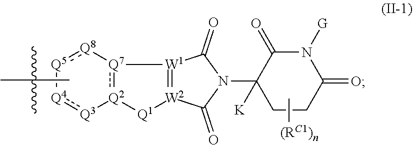
wherein: [symbol]
represents a single bond or a double bond;
W1 and W2 are each independently selected from C, CRC2 and N;
G is selected from the group consisting of H, OH, CH2OH, RC3OCOORC4, RC3OCONRC4RC5, and 2-(trimethylsilyl)ethoxymethyl group;
Q1 to Q7 are each independently C, O, S, N, CRC2 or NRC2; at least one of W1, W2, Q1, Q2, Q3, Q4, Q5, Q6 and Q7 comprises a heteroatom;
K is selected from the group consisting of H, an unsubstituted alkyl group, an alkyl group substituted by RC2, an unsubstituted cycloalkyl group, and a cycloalkyl group substituted by RC2;
K is bound to the 6-membered ring with a stereospecific bond or a non-stereospecific bond;
RC1 is selected from the group consisting of an unsubstituted alkyl group, an alkyl group substituted by RC2, an unsubstituted aryl group, an aryl group substituted by RC2, an unsubstituted alkyl-aryl group, an alkyl-aryl group substituted by RC2, an unsubstituted alkoxy group, and an alkoxy group substituted by RC2;
RC2 is selected from the group consisting of H, halo, CH2OH, CRC4, NRC4RC5, 2-(trimethylsilyl)ethoxymethyl, an alkoxyl group, an unsubstituted alkyl group, an alkyl group substituted by one or more halo groups, an unsubstituted cycloalkyl group, a cycloalkyl group substituted by one or more halo groups, an unsubstituted aryl group, an aryl group substituted by one or more halo groups, an unsubstituted heteroaryl group, a heteroaryl group substituted by one or more halo groups, an unsubstituted heterocyclyl group, and a heterocyclyl group substituted by one or more halo groups;
RC3 is selected from the group consisting of an unsubstituted alkylene group, and an alkylene group substituted by RC2;
RC4 and RC5 are independently selected from the group consisting of an unsubstituted alkyl group, an alkyl group substituted by RC2, an unsubstituted cycloalkyl group, a cycloalkyl group substituted by RC2, an unsubstituted heterocyclyl group, a heterocyclyl group substituted by RC2, an unsubstituted aryl group, an aryl group substituted by RC2, an unsubstituted heteroaryl group, and a heteroaryl group substituted by RC2; and n is 0, 1, 2, 3 or 4, or a pharmaceutically acceptable salt or analog thereof, wherein the TBM is not selected from the group consisting of:

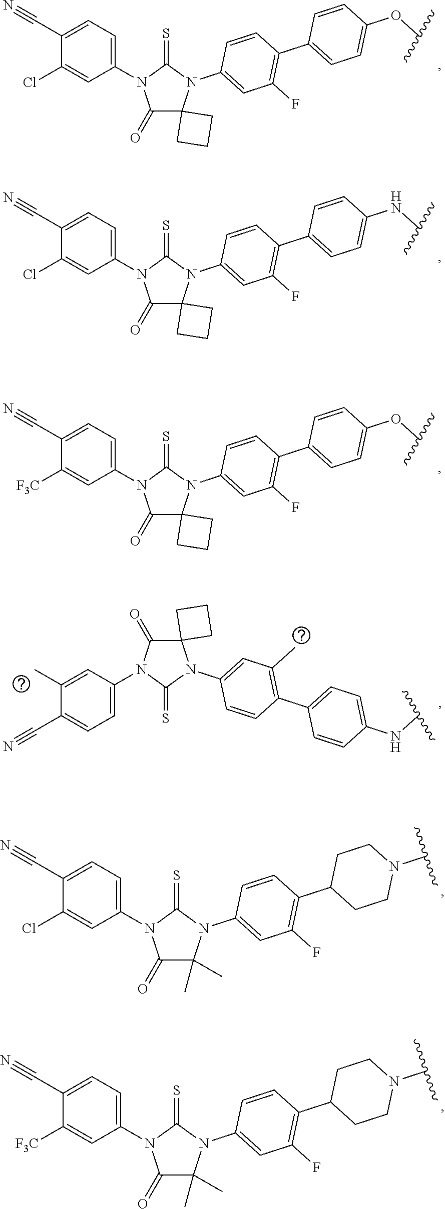 [SMILES: *Oc1ccc(-c2ccc(N3C(=S)N(c4ccc(C#N)c(Cl)c4)C(=O)C34CCC4)cc2F)cc1 ; *Nc1ccc(-c2ccc(N3C(=S)N(c4ccc(C#N)c(Cl)c4)C(=O)C34CCC4)cc2F)cc1 ; *Oc1ccc(-c2ccc(N3C(=S)N(c4ccc(C#N)c(C(F)(F)F)c4)C(=O)C34CCC4)cc2F)cc1 ; *Nc1ccc(-c2ccc(N3C(=S)N(c4ccc(C#N)c(C)c4)C(=O)C34CCC4)cc2C)cc1 ; *N1CCC(c2ccc(N3C(=S)N(c4ccc(C#N)c(Cl)c4)C(=O)C3(C)C)cc2F)CC1 ; *N1CCC(c2ccc(N3C(=S)N(c4ccc(C#N)c(C(F)(F)F)c4)C(=O)C3(C)C)cc2F)CC1]

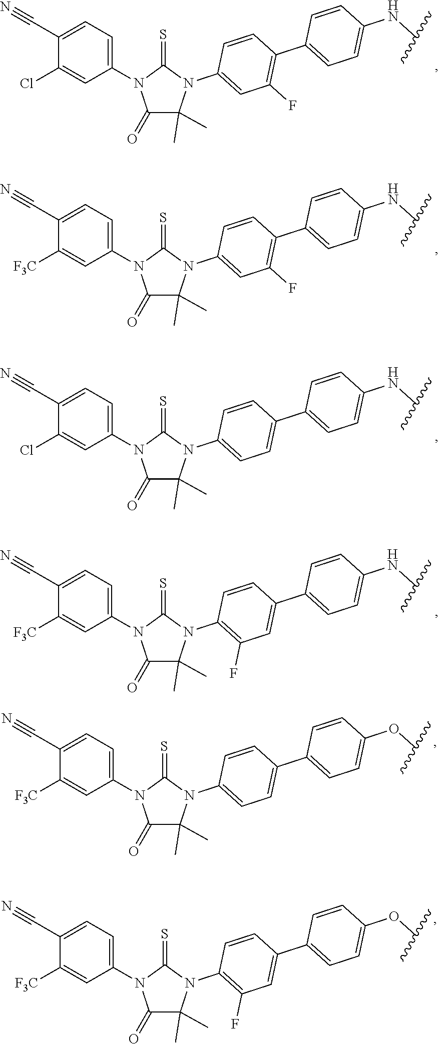 [SMILES: *Nc1ccc(-c2ccc(N3C(=S)N(c4ccc(C#N)c(Cl)c4)C(=O)C3(C)C)cc2F)cc1 ; *Nc1ccc(-c2ccc(N3C(=S)N(c4ccc(C#N)c(C(F)(F)F)c4)C(=O)C3(C)C)cc2F)cc1 ; *Nc1ccc(-c2ccc(N3C(=S)N(c4ccc(C#N)c(Cl)c4)C(=O)C3(C)C)cc2)cc1 ; *Nc1ccc(-c2ccc(N3C(=S)N(c4ccc(C#N)c(C(F)(F)F)c4)C(=O)C3(C)C)c(F)c2)cc1 ; *Oc1ccc(-c2ccc(N3C(=S)N(c4ccc(C#N)c(C(F)(F)F)c4)C(=O)C3(C)C)cc2)cc1 ; *Oc1ccc(-c2ccc(N3C(=S)N(c4ccc(C#N)c(C(F)(F)F)c4)C(=O)C3(C)C)c(F)c2)cc1]

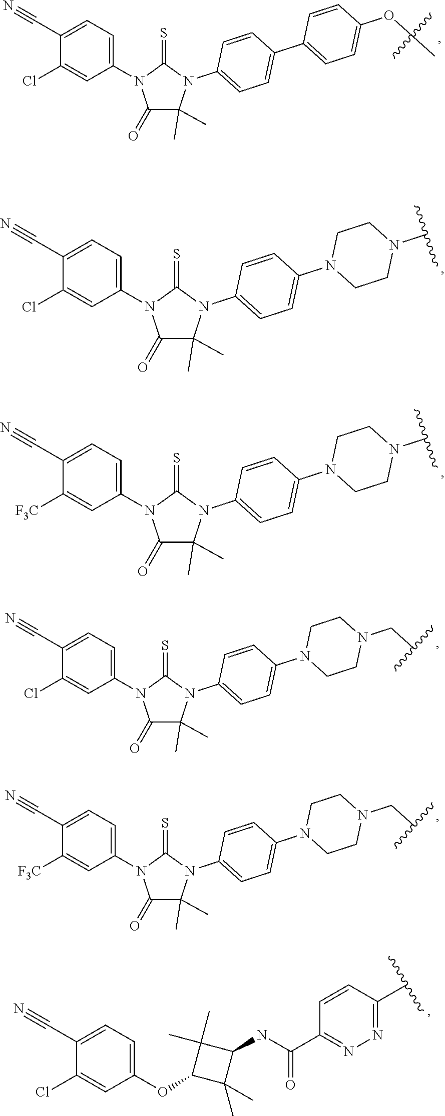 [SMILES: *Oc1ccc(-c2ccc(N3C(=S)N(c4ccc(C#N)c(Cl)c4)C(=O)C3(C)C)cc2)cc1 ; *N1CCN(c2ccc(N3C(=S)N(c4ccc(C#N)c(Cl)c4)C(=O)C3(C)C)cc2)CC1 ; *N1CCN(c2ccc(N3C(=S)N(c4ccc(C#N)c(C(F)(F)F)c4)C(=O)C3(C)C)cc2)CC1 ; *CN1CCN(c2ccc(N3C(=S)N(c4ccc(C#N)c(Cl)c4)C(=O)C3(C)C)cc2)CC1 ; *CN1CCN(c2ccc(N3C(=S)N(c4ccc(C#N)c(C(F)(F)F)c4)C(=O)C3(C)C)cc2)CC1 ; *c1ccc(C(=O)N[C@H]2C(C)(C)[C@H](Oc3ccc(C#N)c(Cl)c3)C2(C)C)nn1]

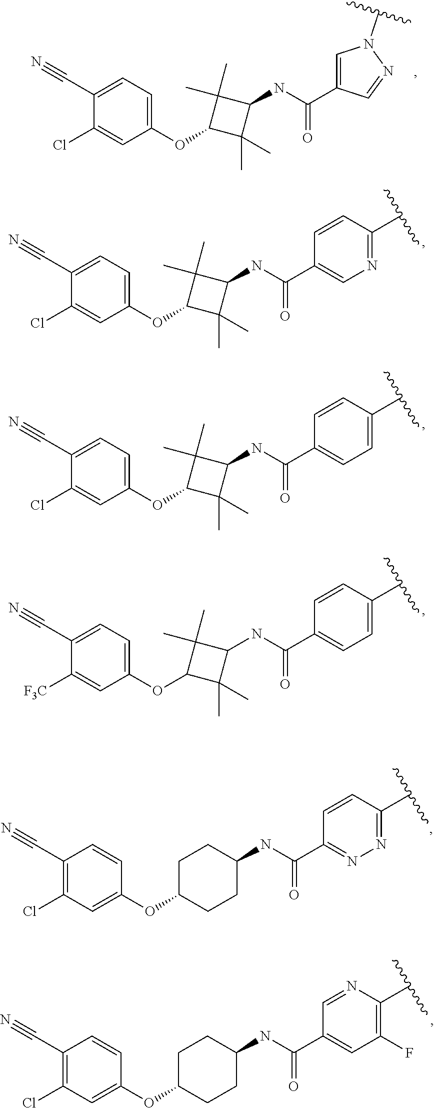 [SMILES: *n1cc(C(=O)N[C@H]2C(C)(C)[C@H](Oc3ccc(C#N)c(Cl)c3)C2(C)C)cn1 ; *c1ccc(C(=O)N[C@H]2C(C)(C)[C@H](Oc3ccc(C#N)c(Cl)c3)C2(C)C)cn1 ; *c1ccc(C(=O)N[C@H]2C(C)(C)[C@H](Oc3ccc(C#N)c(Cl)c3)C2(C)C)cc1 ; *c1ccc(C(=O)NC2C(C)(C)C(Oc3ccc(C#N)c(C(F)(F)F)c3)C2(C)C)cc1 ; *c1ccc(C(=O)N[C@H]2CC[C@H](Oc3ccc(C#N)c(Cl)c3)CC2)nn1 ; *c1ncc(C(=O)N[C@H]2CC[C@H](Oc3ccc(C#N)c(Cl)c3)CC2)cc1F]

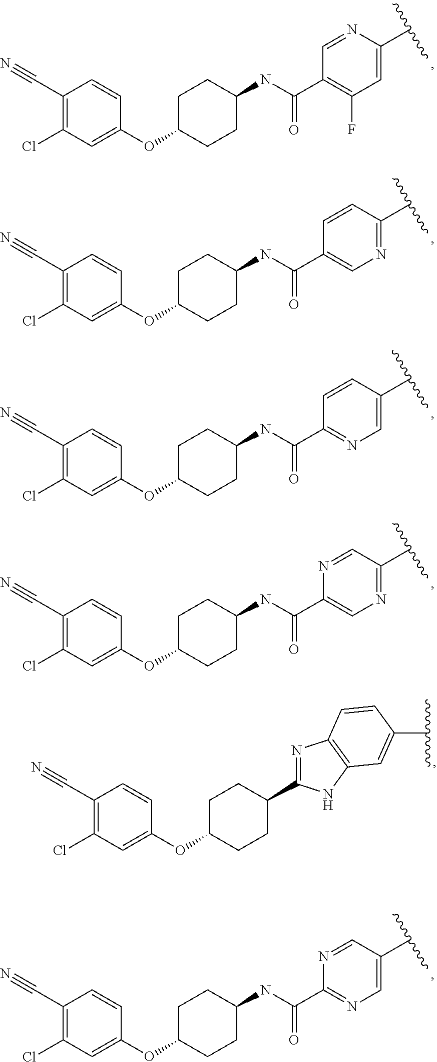 [SMILES: *c1cc(F)c(C(=O)N[C@H]2CC[C@H](Oc3ccc(C#N)c(Cl)c3)CC2)cn1 ; *c1ccc(C(=O)N[C@H]2CC[C@H](Oc3ccc(C#N)c(Cl)c3)CC2)cn1 ; *c1ccc(C(=O)N[C@H]2CC[C@H](Oc3ccc(C#N)c(Cl)c3)CC2)nc1 ; *c1cnc(C(=O)N[C@H]2CC[C@H](Oc3ccc(C#N)c(Cl)c3)CC2)cn1 ; *c1ccc2nc([C@H]3CC[C@H](Oc4ccc(C#N)c(Cl)c4)CC3)[nH]c2c1 ; *c1cnc(C(=O)N[C@H]2CC[C@H](Oc3ccc(C#N)c(Cl)c3)CC2)nc1]

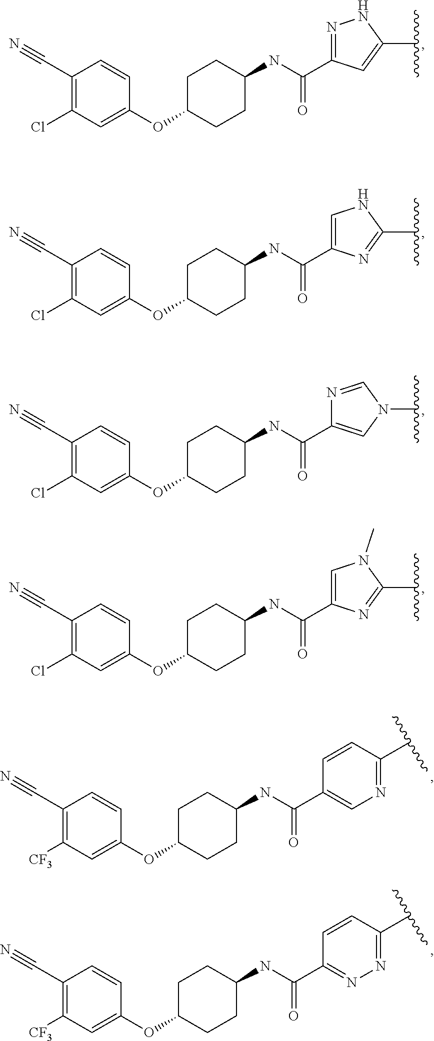 [SMILES: *c1cc(C(=O)N[C@H]2CC[C@H](Oc3ccc(C#N)c(Cl)c3)CC2)n[nH]1 ; *c1nc(C(=O)N[C@H]2CC[C@H](Oc3ccc(C#N)c(Cl)c3)CC2)c[nH]1 ; *n1cnc(C(=O)N[C@H]2CC[C@H](Oc3ccc(C#N)c(Cl)c3)CC2)c1 ; *c1nc(C(=O)N[C@H]2CC[C@H](Oc3ccc(C#N)c(Cl)c3)CC2)cn1C ; *c1ccc(C(=O)N[C@H]2CC[C@H](Oc3ccc(C#N)c(C(F)(F)F)c3)CC2)cn1 ; *c1ccc(C(=O)N[C@H]2CC[C@H](Oc3ccc(C#N)c(C(F)(F)F)c3)CC2)nn1]

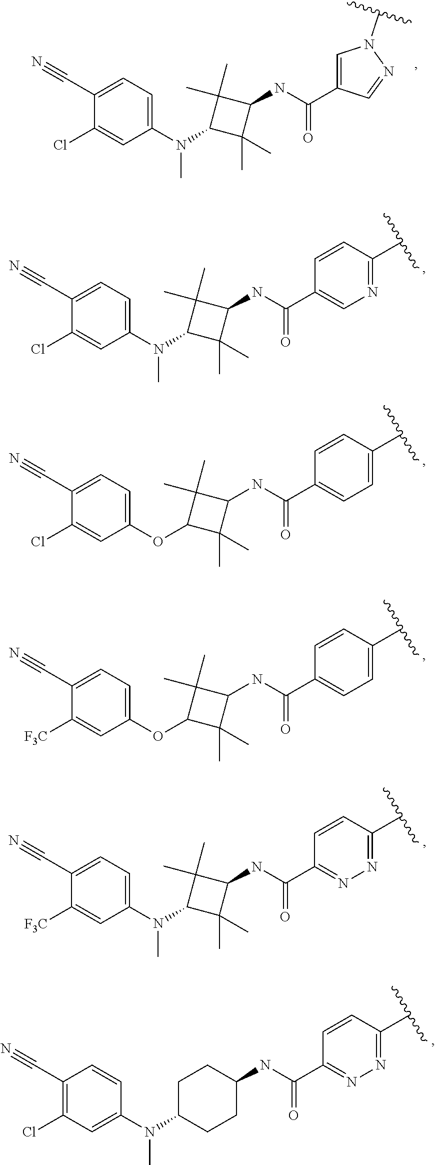 [SMILES: *n1cc(C(=O)N[C@H]2C(C)(C)[C@H](N(C)c3ccc(C#N)c(Cl)c3)C2(C)C)cn1 ; *c1ccc(C(=O)N[C@H]2C(C)(C)[C@H](N(C)c3ccc(C#N)c(Cl)c3)C2(C)C)cn1 ; *c1ccc(C(=O)NC2C(C)(C)C(Oc3ccc(C#N)c(Cl)c3)C2(C)C)cc1 ; *c1ccc(C(=O)NC2C(C)(C)C(Oc3ccc(C#N)c(C(F)(F)F)c3)C2(C)C)cc1 ; *c1ccc(C(=O)N[C@H]2C(C)(C)[C@H](N(C)c3ccc(C#N)c(C(F)(F)F)c3)C2(C)C)nn1 ; *c1ccc(C(=O)N[C@H]2CC[C@H](N(C)c3ccc(C#N)c(Cl)c3)CC2)nn1]

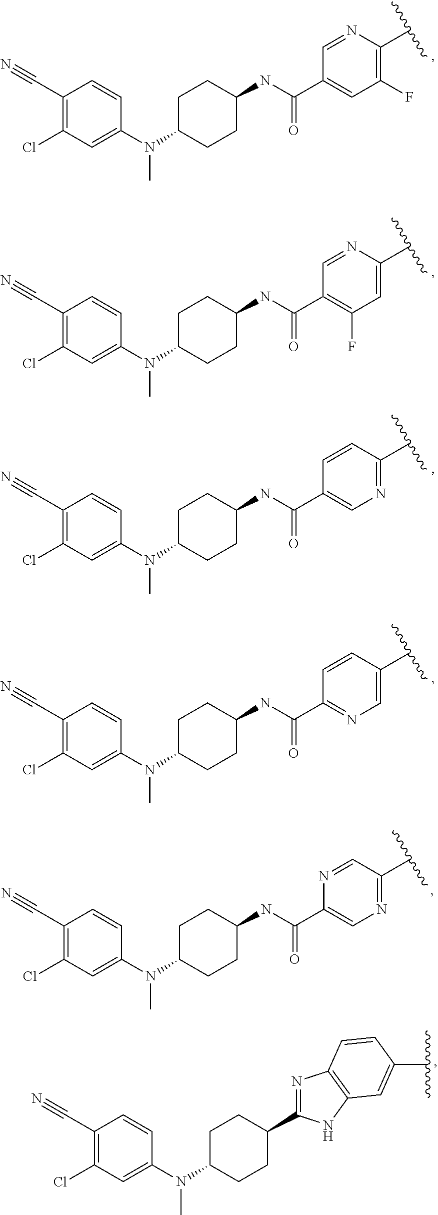 [SMILES: *c1ncc(C(=O)N[C@H]2CC[C@H](N(C)c3ccc(C#N)c(Cl)c3)CC2)cc1F ; *c1cc(F)c(C(=O)N[C@H]2CC[C@H](N(C)c3ccc(C#N)c(Cl)c3)CC2)cn1 ; *c1ccc(C(=O)N[C@H]2CC[C@H](N(C)c3ccc(C#N)c(Cl)c3)CC2)cn1 ; *c1ccc(C(=O)N[C@H]2CC[C@H](N(C)c3ccc(C#N)c(Cl)c3)CC2)nc1 ; *c1cnc(C(=O)N[C@H]2CC[C@H](N(C)c3ccc(C#N)c(Cl)c3)CC2)cn1 ; *c1ccc2nc([C@H]3CC[C@H](N(C)c4ccc(C#N)c(Cl)c4)CC3)[nH]c2c1]

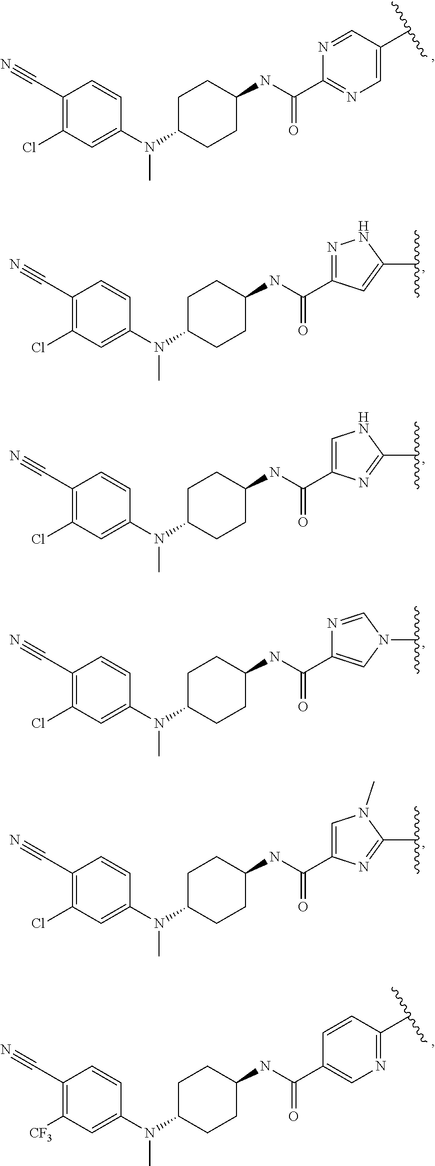 [SMILES: *c1cnc(C(=O)N[C@H]2CC[C@H](N(C)c3ccc(C#N)c(Cl)c3)CC2)nc1 ; *c1cc(C(=O)N[C@H]2CC[C@H](N(C)c3ccc(C#N)c(Cl)c3)CC2)n[nH]1 ; *c1nc(C(=O)N[C@H]2CC[C@H](N(C)c3ccc(C#N)c(Cl)c3)CC2)c[nH]1 ; *n1cnc(C(=O)N[C@H]2CC[C@H](N(C)c3ccc(C#N)c(Cl)c3)CC2)c1 ; *c1nc(C(=O)N[C@H]2CC[C@H](N(C)c3ccc(C#N)c(Cl)c3)CC2)cn1C ; *c1ccc(C(=O)N[C@H]2CC[C@H](N(C)c3ccc(C#N)c(C(F)(F)F)c3)CC2)cn1]

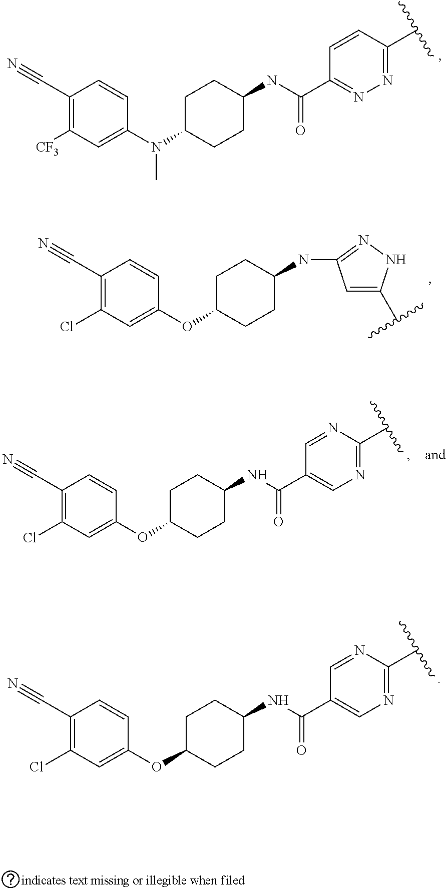 [SMILES: *c1ccc(C(=O)N[C@H]2CC[C@H](N(C)c3ccc(C#N)c(C(F)(F)F)c3)CC2)nn1 ; *c1cc(N[C@H]2CC[C@H](Oc3ccc(C#N)c(Cl)c3)CC2)n[nH]1 ; *c1ncc(C(=O)N[C@H]2CC[C@H](Oc3ccc(C#N)c(Cl)c3)CC2)cn1 ; *c1ncc(C(=O)N[C@H]2CC[C@@H](Oc3ccc(C#N)c(Cl)c3)CC2)cn1]

2. The bivalent compound of claim 1, wherein the TBM is not selected from the group consisting of: 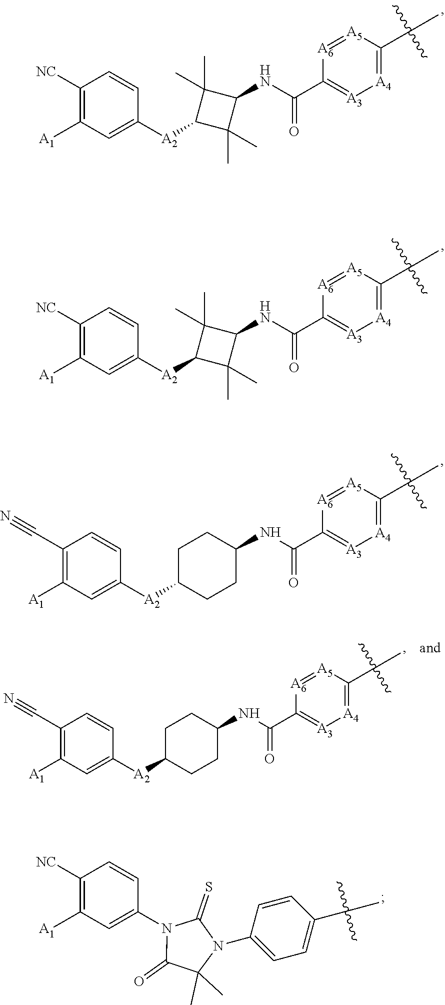
wherein A1 is selected from Cl, F, Br or CF3; A2 is selected from O, NH, N-methyl or N-ethyl; and A3, A4, A5 and A6 are each independently CH or N.

3. The bivalent compound of claim 1, wherein the TBM is not selected from the group consisting of: 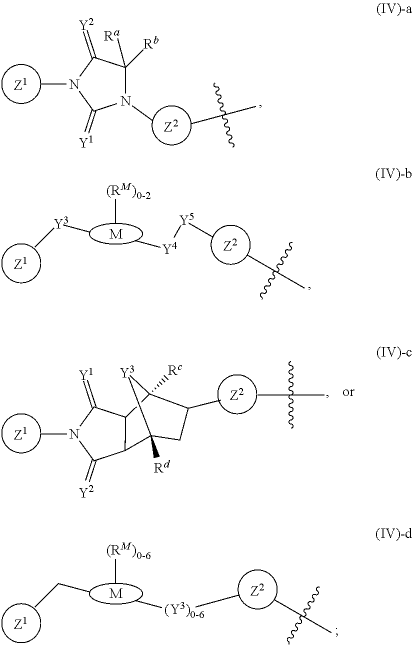
wherein Z1 is selected from the group consisting of an aryl group, a heteroaryl group, a bicyclic group, and a bi-heterocyclic group, each independently substituted by one or more substituents selected from the group consisting of a halo group, a hydroxyl group, a nitro group, CN, C≡CH, an unsubstituted C1-6 alkyl group, a C1-6 alkyl group substituted by a C1-6 alkoxyl group or one or more halo groups, an unsubstituted C1-6 alkoxyl group, a C1-6 alkoxyl group substituted by one or more halo groups, an unsubstituted C2-6 alkenyl, a C2-6 alkenyl substituted by one or more halo groups, an unsubstituted C2-6 alkynyl, and a C3-6 alkynyl substituted by one or more halo groups;
Y1, Y2, Y6 are each independently NRY1, O or S;
Y3, Y4, Y5 are each independently selected from the group consisting of a bond, O, NRY2, CRY1RY2, C═O, C═S, SO, SO2, a heteroaryl group, and an aryl group;
M is a 3- to 6-membered ring with 0 to 4 heteroatoms, which is unsubstituted or substituted by 1 to 6 RM groups;
each RM group is independently selected from the group consisting of H, an unsubstituted C1-6 alkyl group, a C1-6 alkyl group substituted by a C1-6 alkoxyl group or one or more halo groups, halogen, and a C1-6 alkoxy group; or two RM groups are taken together with the atom they are attached to and form a 3- to 8-membered ring system containing 0 to 2 heteroatoms;
Ra, Rb, Rc, Rd, RY1, RY2 are each independently selected from the group consisting of H, an unsubstituted C1-6 alkyl group, a C1-6 alkyl group substituted by a C1-6 alkoxyl group or one or more halo groups, halogen, a C1-6 alkoxy group, a cyclic group, and a heterocyclic group; or Ra, Rb are taken together with the atom they are attached to and form a 3- to 8-membered ring system containing 0 to 2 heteroatoms;
Z2 is selected from the group consisting of a bond, a C1-6 alkyl group, a C1-6 heteroalkyl group, O, an aryl group, a heteroaryl group, an alicyclic group, a heterocyclic group, a biheterocyclic group, a biaryl group, and a biheteroaryl group, each of which is unsubstituted or substituted by 1 to 10 RZ2 groups;
each R^{Z2} group is independently selected from the group consisting of H, halo, an unsubstituted C1-6 alkyl group, a C1-6 alkyl group substituted by one or more F, —ORZ2A, a C3-6 cycloalkyl group, a C4-6 cycloheteroalkyl group, an unsubstituted C1-6 alkyl group, a C1-6 alkyl group substituted by a C1-3 alkyl group or a C1-6 alkoxyl group or one or more halo groups, an unsubstituted heterocyclic group, a heterocyclic group substituted by a C1-3 alkyl group or a C1-6 alkoxyl group or one or more halo groups, an unsubstituted aryl group, an aryl group substituted by a C1-3 alkyl group or a C1-6 alkoxyl group or one or more halo groups, an unsubstituted heteroaryl group, a heteroaryl group substituted by a C1-3 alkyl group or a C1-6 alkoxyl group or one or more halo groups, a bicyclic hereoaryl group, an unsubstituted OC₁₋₃ alkyl group, and a OC₁₋₃ alkyl substituted by one or more groups selected from F, OH, NH₂, NRY1RY2 and CN; and
R^{Z2A} is selected from the group consisting of H, a C1-6 alkyl group, and a C1-6 heteroalkyl group, each of which is unsubstituted or substituted by a cycloalkyl group, a cycloheteroalkyl group, an aryl group, a heterocyclic group, a heteroaryl group, halo, or a OC1-3 alkyl group.

4. The bivalent compound of claim 1, wherein the TBM is capable of binding to a protein degradable by a cereblon E3 ubiquitin ligase.

5. The bivalent compound of claim 1, wherein the CLM is represented with Formula (II)-2: 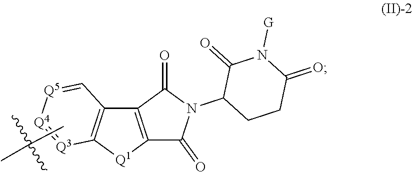
wherein
Q1 is O, S or NRC2;
Q3 to Q5 are each independently C or CRC2;
G is selected from the group consisting of H, OH, CH2OH, RC3OCOORC4 and 2-(trimethylsilyl)ethoxymethyl group;
RC2 is selected from the group consisting of H, CH2OH, CRC4, 2-(trimethylsilyl)ethoxymethyl, an unsubstituted C1-6 alkyl group, a C1-6 alkyl group substituted by one or more halo groups, a unsubstituted C1-6 cycloalkyl group, a C1-6 cycloalkyl group substituted by one or more halo groups;
RC3 is selected from the group consisting of a methylene group and a methylene group substituted by RC2;
RC4 is selected from the group consisting of an unsubstituted alkyl group, an alkyl group substituted by RC2, an unsubstituted cycloalkyl group, a cycloalkyl group substituted by RC2, an unsubstituted heterocyclyl group, a heterocyclyl group substituted by RC2.

6. The bivalent compound of claim 5, wherein Q1 is NRC2.

7. The bivalent compound of claim 6, wherein RC2 is an unsubstituted C1-6 alkyl group, a C1-6 alkyl group substituted by one or more halo groups, an unsubstituted C1-6 cycloalkyl group, a C1-6 cycloalkyl group substituted by one or more halo groups.

8. The bivalent compound of claim 6, wherein RC2 is methyl or ethyl.

9. The bivalent compound of claim 5, wherein Q3-Q5 are each C.

10. The bivalent compound of claim 5, wherein G is selected from the group consisting of H, CH2OH, and RC3OCOORC4.

11. The bivalent compound of claim 5, wherein G is H.

12. The bivalent compound of claim 5, wherein G is CH2OH.

13. The bivalent compound of claim 5, wherein G is RC3OCOORC4.

14. The bivalent compound of claim 13, wherein RC3 is selected from the group consisting of an unsubstituted alkylene group.

15. The bivalent compound of claim 13, wherein RC3 is methylene or ethylene.

16. The bivalent compound of claim 13, wherein RC3 is methylene.

17. The bivalent compound of claim 13, wherein RC4 is an unsubstituted alkyl group or an alkyl group substituted by RC2.

18. The bivalent compound of claim 13, wherein RC4 is an unsubstituted alkyl group.

19. The bivalent compound of claim 13, wherein RC4 is an unsubstituted C1-4 alkyl group.

20. The bivalent compound of claim 13, wherein RC4 is methyl or ethyl.

21. The bivalent compound of claim 1, wherein the TBM is connected to the CLM through a linker moiety (L).

22. The bivalent compound of claim 21, wherein the TBM is connected to the CLM through Q4.

23. The bivalent compound of claim 21, wherein the linker moiety is of Formula (III): 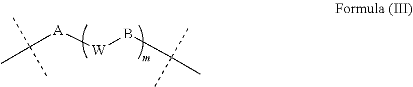
wherein
A, W, and B, at each occurrence, are independently selected from null, CO, CO₂, C(O)NR¹, C(S)NR¹, O, S, SO, SO₂, SO₂NR¹, NR¹, NR¹CO, NR¹CONR², NR¹C(S), optionally substituted C₁-C₈ alkyl, optionally substituted C₁-C₈ alkoxy, optionally substituted C₁-C₈alkoxyC₁-C₈alkyl, optionally substituted C₁-C₈ haloalkyl, optionally substituted C₁-C₈ hydroxyalkyl, optionally substituted C₂-C₈ alkenyl, optionally substituted C₂-C₈ alkynyl, optionally substituted C₃-C₈ cycloalkyl, optionally substituted C₃-C₈ cycloalkoxy, optionally substituted 3-8 membered heterocyclyl, optionally substituted aryl, optionally substituted heteroaryl, optionally substituted C₃-C₁₃ fused cycloalkyl, optionally substituted C₃-C₁₃ fused heterocyclyl, optionally substituted C₃-C₁₃ bridged cycloalkyl, optionally substituted C₃-C₁₃ bridged heterocyclyl, optionally substituted C₃-C₁₃ spiro cycloalkyl, and optionally substituted C₃-C₁₃ spiro heterocyclyl, wherein
R¹ and R² are independently selected from hydrogen, optionally substituted C₁-C₆ alkyl, optionally substituted C₃-C₆ cycloalkyl, optionally substituted C₃-C₆ cycloalkoxy, optionally substituted 3-6 membered heterocyclyl, optionally substituted C₁-C₆ alkoxy, optionally substituted C₁-C₆ alkoxyalkyl, optionally substituted C₁-C₆ haloalkyl, optionally substituted C₁-C₆ hydroxyalkyl, optionally substituted C₁-C₆ alkylamino, and optionally substituted C₁-C₆alkylaminoC₁-C₆alkyl; and m is 0 to 15.

24. The bivalent compound of claim 21, wherein the linker moiety is of Formula (III)-1: 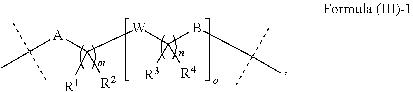
wherein
R¹, R², R³, and R⁴, at each occurrence, are independently selected from hydrogen, halogen, CN, OH, NH₂, optionally substituted C₁-C₆ alkyl, optionally substituted C₃-C₆ cycloalkyl, optionally substituted C₃-C₆ cycloalkoxy, optionally substituted 3-6 membered heterocyclyl, optionally substituted C₁-C₆ alkoxy, optionally substituted C₁-C₆ alkoxyalkyl, optionally substituted C₁-C₆ haloalkyl, optionally substituted C₁-C₆ hydroxyalkyl, optionally substituted C₁-C₆ alkylamino, and optionally substituted C₁-C₆alkylaminoC₁-C₆alkyl;
A, W, and B, at each occurrence, are independently selected from null, CO, CO₂, C(O)NR⁵, C(S)NR⁵, O, S, SO, SO₂, SO₂NR⁵, NR⁵, NR⁵CO, NR⁵CONR⁶, NR⁵C(S), optionally substituted C₁-C₈ alkyl, optionally substituted C₁-C₈ alkoxy, optionally substituted C₁-C₈alkoxyC₁-C₈alkyl, optionally substituted C₁-C₈ haloalkyl, optionally substituted C₁-C₈ hydroxyalkyl, optionally substituted C₂-C₈ alkenyl, optionally substituted C₂-C₈ alkynyl, optionally substituted C₃-C₈ cycloalkyl, optionally substituted C₃-C₈ cycloalkoxy, optionally substituted 3-8 membered heterocyclyl, optionally substituted aryl, optionally substituted heteroaryl, optionally substituted C₃-C₁₃ fused cycloalkyl, optionally substituted C₃-C₁₃ fused heterocyclyl, optionally substituted C₃-C₁₃ bridged cycloalkyl, optionally substituted C₃-C₁₃ bridged heterocyclyl, optionally substituted C₃-C₁₃ spiro cycloalkyl, and optionally substituted C₃-C₁₃ spiro heterocyclyl, wherein
R⁵ and R⁶ are independently selected from hydrogen, optionally substituted C₁-C₆ alkyl, optionally substituted C₃-C₆ cycloalkyl, optionally substituted C₃-C₆ cycloalkoxy, optionally substituted 3-6 membered heterocyclyl, optionally substituted C₁-C₆ alkoxy, optionally substituted C₁-C₆ alkoxyalkyl, optionally substituted C₁-C₆ haloalkyl, optionally substituted C₁-C₆ hydroxyalkyl, optionally substituted C₁-C₆ alkylamino, and optionally substituted C₁-C₆alkylaminoC₁-C₆alkyl;
m is 0 to 15;
each n is 0 to 15; and
o is 0 to 15.

25. The bivalent compound of claim 21, wherein the linker moiety is of Formula (III)-2: 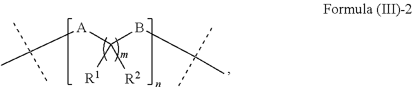
wherein
each R¹, and each R² are independently selected from hydrogen, halogen, CN, OH, NH₂, and optionally substituted C₁-C₆ alkyl, optionally substituted C₃-C₆ cycloalkyl, optionally substituted C₃-C₆ cycloalkoxy, optionally substituted 3-6 membered heterocyclyl, optionally substituted C₁-C₆ alkoxy, optionally substituted C₁-C₆ alkoxyalkyl, optionally substituted C₁-C₆ haloalkyl, optionally substituted C₁-C₆ hydroxyalkyl, optionally substituted C₁-C₆ alkylamino, or C₁-C₆alkylaminoC₁-C₆alkyl;
each A and each B are independently selected from null, CO, CO₂, C(O)NR³, C(S)NR³, O, S, SO, SO₂, SO₂NR³, NR³, NR³CO, NR³CONR⁴, NR³C(S), and optionally substituted C₁-C₈ alkyl, optionally substituted C₁-C₈ alkoxy, optionally substituted C₁-C₈alkoxyC₁-C₈alkyl, optionally substituted C₁-C₈ haloalkyl, optionally substituted C₁-C₈ hydroxyalkyl, optionally substituted C₂-C₈ alkenyl, optionally substituted C₂-C₈ alkynyl, optionally substituted C₃-C₈ cycloalkyl, optionally substituted C₃-C₈ cycloalkoxy, optionally substituted 3-8 membered heterocyclyl, optionally substituted aryl, optionally substituted heteroaryl, optionally substituted C₃-C₁₃ fused cycloalkyl, optionally substituted C₃-C₁₃ fused heterocyclyl, optionally substituted C₃-C₁₃ bridged cycloalkyl, optionally substituted C₃-C₁₃ bridged heterocyclyl, optionally substituted C₃-C₁₃ spiro cycloalkyl, or C₃-C₁₃ spiro heterocyclyl, wherein
R³ and R⁴ are independently selected from hydrogen, and optionally substituted C₁-C₆ alkyl, optionally substituted C₃-C₆ cycloalkyl, optionally substituted C₃-C₆ cycloalkoxy, optionally substituted 3-6 membered heterocyclyl, optionally substituted C₁-C₆ alkoxy, optionally substituted C₁-C₆ alkoxyalkyl, optionally substituted C₁-C₆ haloalkyl, optionally substituted C₁-C₆ hydroxyalkyl, optionally substituted C₁-C₆ alkylamino, or C₁-C₆alkylaminoC₁-C₆alkyl;
each m is 0 to 15; and
n is 0 to 15.

26. The bivalent compound of claim 21, wherein the linker moiety is of FORMULA (III)-3: 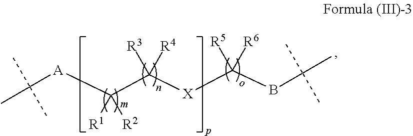
wherein
X is selected from O, NH, and NR⁷;
R¹, R², R³, R⁴, R⁵, and R⁶, at each occurrence, are independently selected from hydrogen, halogen, CN, OH, NH₂, optionally substituted C₁-C₆ alkyl, optionally substituted C₃-C₆ cycloalkyl, optionally substituted C₃-C₆ cycloalkoxy, optionally substituted 3-6 membered heterocyclyl, optionally substituted C₁-C₆ alkoxy, optionally substituted C₁-C₆ alkoxyalkyl, optionally substituted C₁-C₆ haloalkyl, optionally substituted C₁-C₆ hydroxyalkyl, optionally substituted C₁-C₆ alkylamino, and optionally substituted C₁-C₆alkylaminoC₁-C₆alkyl;
A and B, at each occurrence, are independently selected from null, CO, CO₂, C(O)NR⁷, C(S)NR⁷, O, S, SO, SO₂, SO₂NR⁷, NR⁷, NR⁷CO, NR⁷CONR⁸, NR⁷C(S), optionally substituted C₁-C₈ alkyl, optionally substituted C₁-C₈ alkoxy, optionally substituted C₁-C₈alkoxyC₁-C₈alkyl, optionally substituted C₁-C₈ haloalkyl, optionally substituted C₁-C₈ hydroxyalkyl, optionally substituted C₂-C₈ alkenyl, optionally substituted C₂-C₈ alkynyl, optionally substituted C₃-C₈ cycloalkyl, optionally substituted C₃-C₈ cycloalkoxy, optionally substituted 3-8 membered heterocyclyl, optionally substituted aryl, optionally substituted heteroaryl, optionally substituted C₃-C₁₃ fused cycloalkyl, optionally substituted C₃-C₁₃ fused heterocyclyl, optionally substituted C₃-C₁₃ bridged cycloalkyl, optionally substituted C₃-C₁₃ bridged heterocyclyl, optionally substituted C₃-C₁₃ spiro cycloalkyl, and optionally substituted C₃-C₁₃ spiro heterocyclyl, wherein R⁷ and R⁸ are independently selected from hydrogen, optionally substituted C₁-C₆ alkyl, optionally substituted C₃-C₆ cycloalkyl, optionally substituted C₃-C₆ cycloalkoxy, optionally substituted 3-6 membered heterocyclyl, optionally substituted C₁-C₆ alkoxy, optionally substituted C₁-C₆ alkoxyalkyl, optionally substituted C₁-C₆ haloalkyl, optionally substituted C₁-C₆ hydroxyalkyl, optionally substituted C₁-C₆ alkylamino, and optionally substituted C₁-C₆alkylaminoC₁-C₆alkyl;
each m is 0 to 15;
each n is 0 to 15;
o is 0 to 15; and
p is 0 to 15.

27. The bivalent compound of claim 21, wherein the linker moiety is of Formula (IV): 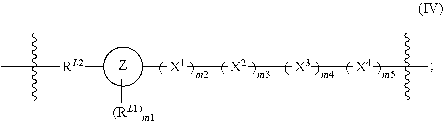
wherein
Z is selected from the group consisting of a 3- to 8-membered ring, a 5- to 12-membered bicyclic ring, an 8- to 15-membered tricyclic ring and a 6- to 12-membered spiro bicyclic ring, each independently having 0-4 heteroatoms;
RL1 is selected from the group consisting of H, an unsubstituted C1-6 alkyl group, a C1-6 alkyl group substituted by a C1-6 alkoxyl group or one or more halo groups, halogen, a C1-6 alkoxy group, a keto group, or an oxide group; wherein, two RL1 groups are optionally taken together to form a 3-8 membered ring system containing 0-2 heteroatoms;
RL2 is a bond or an ethynylene group;
X1 is selected from the group consisting of a methylene group and an ethylene group;
X2 and X4 are independently selected from the group consisting of a methylene group, C(═O), C(═O)C(═O), CONRL3, NRL3C(═O), NRL3C(═O)NRL4, NRL3C(═O)C(═O)NRL4, NRL3SO2NRL4, SO2NRL3, CRL3RL4, NRL5, O and S;
X3 is selected from the group consisting of an unsubstituted C1-8 alkylene group, a C1-8 alkylene group substituted by 1 to 6 RL1, an unsubstituted C1-8 heteroalkylene group, a C1-8 heteroalkylene group substituted by 1 to 6 RL1, an unsubstituted 3- to 8-membered arylene group, a 3- to 8-membered arylene group substituted by 1 to 6 RL1, an unsubstituted 3- to 8-membered heteroarylene group with 1 to 3 hetero atoms, and a 3- to 8-membered heteroarylene group with 1 to 3 hetero atoms and substituted by 1 to 6 RL1, an unsubstituted 3- to 7-membered cyclic alkylene group, a 3- to 7-membered cyclic alkylene group substituted by 1 to 6 RL1, an unsubstituted 3- to 7-membered heterocyclic alkylene group with 1 to 2 hetero atoms, and a 3- to 7-membered heterocyclic alkylene group with 1 to 2 hetero atoms and substituted by 1 to 6 RL1; or, X3 is a 6- to 12-membered spiro bicyclic ring having 0-4 heteroatoms;
RL3 is hydrogen;
RL4 and RL5 are independently selected from the group consisting of H, an unsubstituted C1-6 alkyl group, and a C1-6 alkyl group substituted by a C1-6 alkoxyl group or one or more halo groups;
m1 is 0, 1, 2, 3, 4, 5 or 6;
m2 is 0 or 1;
m3 is 0 or 1;
m4 is 1; and
m5 is 0 or 1.

28. The bivalent compound of claim 21, wherein the linker moiety is selected from the group consisting of: 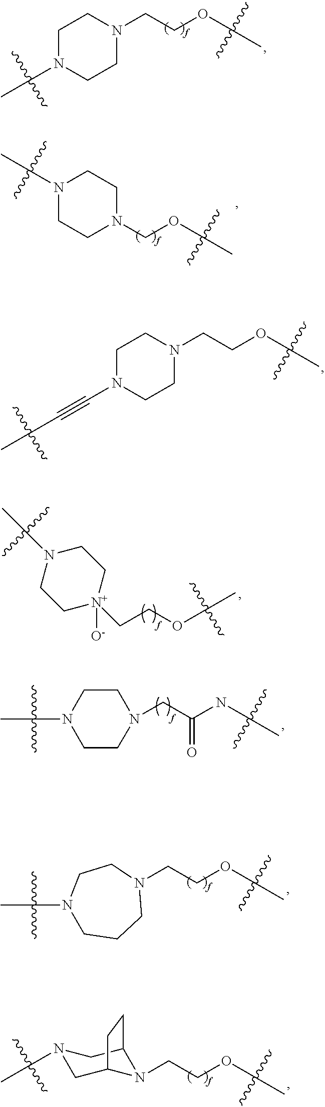 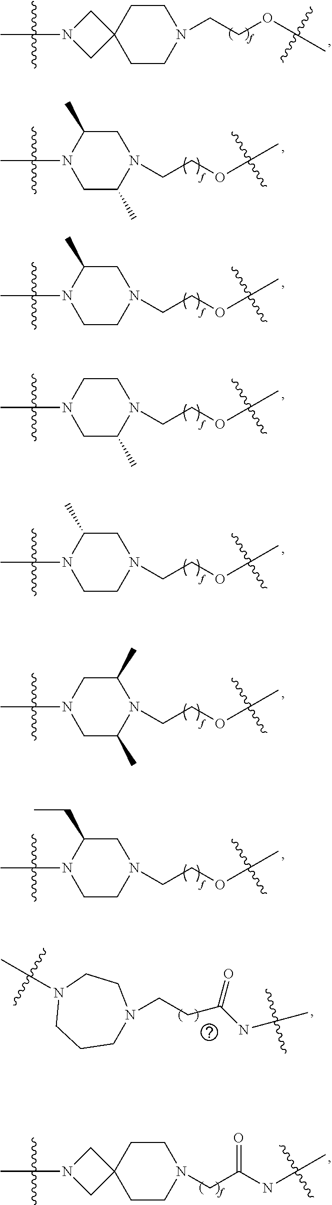 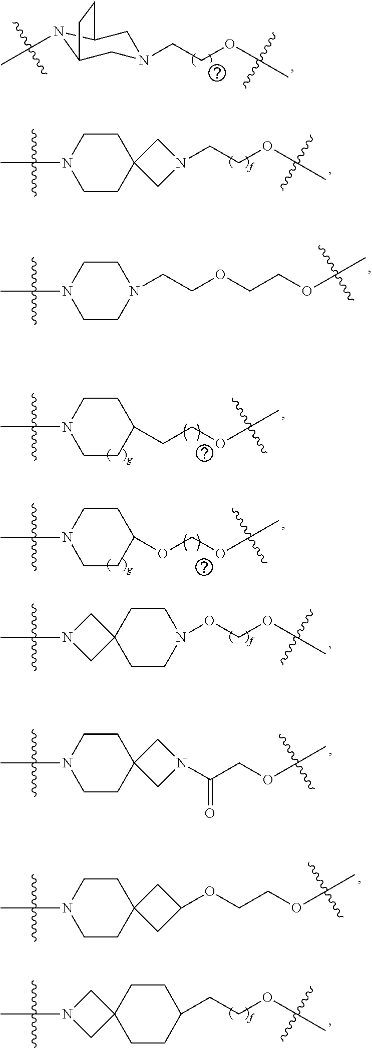 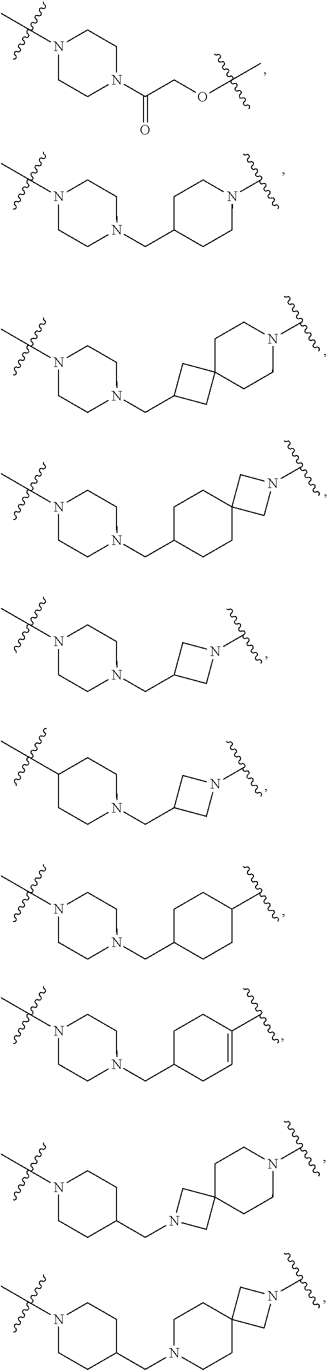 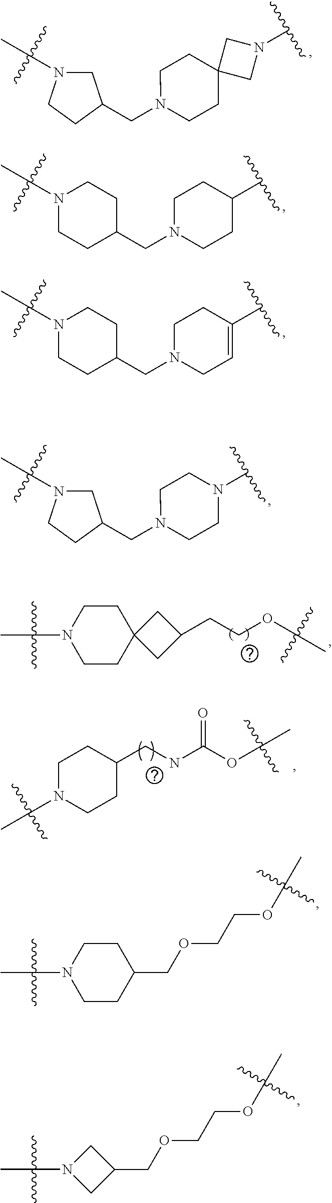 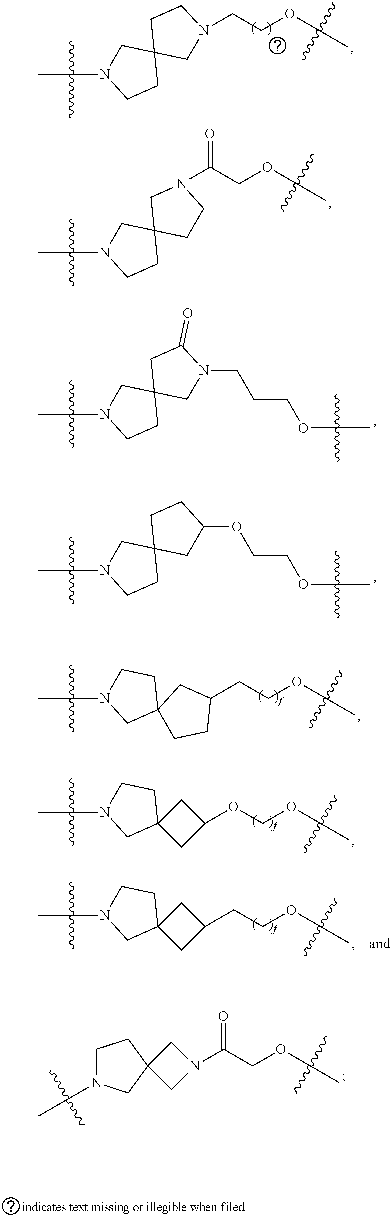
wherein f is an integer of 0, 1, 2, 3 or 4; and g is an integer of 0, 1, 2 or 3.

29. A composition comprising a bivalent compound according to claim 1, and a pharmaceutically acceptable carrier.

30. A method of degrading a protein associated with a disease or condition, by contacting the protein with the bivalent compound according to claim 1.
